(19) United States
(12) Patent Application Publication
(10) Pub. No.: US 2024/0124610 A1
(43) Pub. Date: Apr. 18, 2024

(54) METHODS FOR TREATING HER2-NEGATIVE OR HER2-LOW CANCER

(71) Applicants: Nicole Maher, St. Louis, MO (US); Christopher Maher, St. Louis, MO (US); Andrew Nickless, St. Louis, MO (US); Jin Zhang, St. Louis, MO (US); Amy Ly, St. Louis, MO (US); Jace Webster, St. Louis, MO (US)

(72) Inventors: Nicole Maher, St. Louis, MO (US); Christopher Maher, St. Louis, MO (US); Andrew Nickless, St. Louis, MO (US); Jin Zhang, St. Louis, MO (US); Amy Ly, St. Louis, MO (US); Jace Webster, St. Louis, MO (US)

(73) Assignee: Washington University, St. Louis, MO (US)

(21) Appl. No.: 18/318,232

(22) Filed: May 16, 2023

Related U.S. Application Data
(60) Provisional application No. 63/342,274, filed on May 16, 2022.

(51) Int. Cl. C07K 16/32; A61K 31/506; A61P 35/00; C12Q 1/6886

(52) U.S. Cl. CPC C07K 16/32; A61K 31/506; A61P 35/00; C12Q 1/6886; C12Q 2600/106; C12Q 2600/158

(57) ABSTRACT

The present disclosure provides methods for treating a subject having a HER2 non-amplified (HER2-negative) cancer or low HER2 expressing (HER2-low) cancer, comprising detecting expression of a BCAR4 gene fusion in a biological sample from the subject. If expression of the BCAR4 gene fusion is detected, a HER2-targeted cancer treatment is administered to the subject; if expression of the BCAR4 gene fusion is not detected, a cancer treatment that is not HER2-targeted is administered to the subject. Methods of predicting response to a HER2-targeted cancer treatment and detecting BCAR4 or BCAR4 gene fusion activation are also provided.

### CROSS-REFERENCE TO RELATED APPLICATIONS

This application claims priority from U.S. Provisional Application Ser. No. 63/342,274 filed on 16 May 2022, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “019924-US-NP_Sequence_Listing.xml” created on 16 May 2023; 19,411 bytes). The subject matter of the Sequence Listing is incorporated herein by reference in its entirety.

### FIELD

The present disclosure generally relates to methods of treating cancer.

### SUMMARY

Among the various aspects of the present disclosure, methods of treating a subject having a HER2-negative or HER2-low cancer, methods of predicting response to a HER2-targeted cancer treatment in a subject having a HER2-negative or HER2-low cancer, and methods of detecting BCAR4 or BCAR4 gene fusion activation in a subject having cancer are provided.

In one aspect of the present disclosure, a method of treating a subject having a HER2 non-amplified (HER2-negative) cancer or low HER2 expressing (HER2-low) cancer is provided. The method comprises: providing a biological sample from the subject; detecting expression of a BCAR4 gene fusion in the biological sample; and administering: a HER2-targeted cancer treatment to the subject if expression of the BCAR4 gene fusion is detected, or a cancer treatment that is not HER2-targeted to the subject if expression of the BCAR4 gene fusion is not detected.

In some embodiments, the BCAR4 gene fusion comprises a nucleotide sequence derived from LITAF, ZC3H7A, or a variant thereof; the BCAR4 gene fusion comprises exon 4 of the BCAR4 gene or a variant thereof; and/or the BCAR4 gene fusion comprises a nucleotide sequence encoding a peptide comprising SEQ ID NO: 1, SEQ ID NO: 2, or a variant thereof. In some embodiments, the subject has a HER2-negative or HER2-low cancer selected from the group consisting of stomach cancer, cervical cancer, breast cancer, esophageal cancer, ovarian cancer, skin cancer, bladder cancer, lung cancer, uterine cancer, colon cancer, or prostate cancer. In some embodiments, the subject has HER2-negative or HER2-low breast cancer. In some embodiments, the subject has a luminal A or luminal B breast cancer. In some embodiments, the HER2-targeted cancer treatment comprises lapatinib or trastuzumab.

In another aspect of the present disclosure, a method for predicting response to a HER2-targeted cancer treatment in a subject having a HER2 non-amplified (HER2-negative) cancer or low HER2 expressing (HER2-low) cancer is provided. The method comprises: providing a biological sample from the subject; detecting expression of a BCAR4 gene fusion in the biological sample; and determining: the subject is predicted to be responsive to a HER2-targeted cancer treatment if expression of the BCAR4 gene fusion is detected, or the subject is predicted to be unresponsive to a HER2-targeted cancer treatment if expression of the BCAR4 gene fusion is not detected.

In some embodiments, the BCAR4 gene fusion comprises a nucleotide sequence derived from LITAF, ZC3H7A, or a variant thereof; the BCAR4 gene fusion comprises exon 4 of the BCAR4 gene or a variant thereof; and/or the BCAR4 gene fusion comprises a nucleotide sequence encoding a peptide comprising SEQ ID NO: 1, SEQ ID NO: 2, or a variant thereof. In some embodiments, the subject has a HER2-negative or HER2-low cancer selected from the group consisting of stomach cancer, cervical cancer, breast cancer, esophageal cancer, ovarian cancer, skin cancer, bladder cancer, lung cancer, uterine cancer, colon cancer, or prostate cancer. In some embodiments, the subject has HER2-negative or HER2-low breast cancer. In some embodiments, the HER2-targeted cancer treatment comprises lapatinib or trastuzumab. In some embodiments, if expression of the BCAR4 gene fusion is detected, the subject is further predicted to be resistant to a hormone therapy.

In yet another aspect of the present disclosure, a method of detecting BCAR4 or BCAR4 gene fusion activation in a subject having cancer is provided. The method comprises: providing a biological sample from a subject; isolating cell-free DNA (cfDNA) from the biological sample; and measuring a 5-Hydroxymethylcytosine (5hmC) signal for BCAR4 in the cfDNA using sequencing, wherein the 5hmC signal positively correlates with BCAR4 or BCAR4 gene fusion expression.

In some embodiments, the 5hmC signal is measured at the BCAR4 promoter. In some embodiments, the biological sample comprises plasma. In some embodiments, the subject has a HER2 non-amplified (HER2-negative) cancer or low HER2 expressing (HER2-low) cancer.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic showing analysis of 9,638 patients across 32 solid tumor types revealed an annotated long non-coding RNA (lncRNA), Breast Cancer Anti-Estrogen Resistance 4 (BCAR4) was the most prevalent, uncharacterized downstream gene fusion partner occurring in 11 cancers in accordance with the present disclosure.

FIG. 2A-FIG. 2B is an exemplary embodiment showing pan-cancer discovery of BCAR4 gene fusions in accordance with the present disclosure. FIG. 2A is a dot plot of recurrent gene fusions by number of patients across cancer types. Callout boxes list detected 50 partners of known gene fusions. FIG. 2B is a graph showing structure of expressed BCAR4 fusion transcripts with the 50 gene fusion partners represented in various colors and BCAR4 represented in green (left). Fusions are sorted by descending prevalence. Representation of patients across cancer types expressing BCAR4 fusion transcripts is also shown (right). Rows correspond to the various BCAR4 isoforms. A cell is colored blue if the patient expresses a particular BCAR4 fusion, and the intensity corresponds to the number of RNA-seq reads supporting evidence of the fusion expression. STAD, stomach adenocarcinoma; CESC, cervical squamous cell carcinoma and endocervical adenocarcinoma; BRCA, breast invasive carcinoma; ESCA, esophageal carcinoma; OV, ovarian serous cystadenocarcinoma; SKCM, skin cutaneous melanoma; BLCA, bladder urothelial carcinoma; LUAD, lung adenocarcinoma; UCEC, uterine corpus endometrial carcinoma; CRC, colorectal cancer; PRAD, prostate adenocarcinoma.

FIG. 3 is a schematic of BCAR4 gene fusions in accordance with the present disclosure. FIG. 3 shows endogenous gene fusions found in SNU308 and TUHR14TKB cell lines.

FIG. 4A-FIG. 4B is an exemplary embodiment showing BCAR4 gene fusions alter cell-cycle and proliferation in accordance with the present disclosure. FIG. 4A and FIG. 4B contain representative dot plots of EdU-DNA stain (FxCycle) flow cytometry analysis and quantification of G1, S, G2-M cell populations after siRNA-mediated silencing of BCAR4 fusions in SNU308 (n=3; FIG. 4A) or TUHR14TKB (n=4; FIG. 4B) cells. FIG. 4A and FIG. 4B also contain bar graphs showing qRT-PCR analysis confirmed knockdown of BCAR4 fusions.

FIG. 5 is an exemplary embodiment showing knockdown of BCAR4 gene fusions do not alter cell viability in accordance with the present disclosure. FIG. 5 contains a bar graph quantifying cell death via annexin staining after siRNA-mediated silencing of BCAR4 fusions in SNU308 cells. No statistical significance as determined by paired two-tailed t-test. RT-qPCR analysis confirmed knockdown of BCAR4 fusions. Bar graphs present normalized mean±SEM. n=3

FIG. 6 is a schematic of BCAR4 gene fusions in accordance with the present disclosure. FIG. 6 shows expression plasmids.

FIG. 7A-FIG. 7B is an exemplary embodiment showing BCAR4 gene fusion RNA expression in accordance with the present disclosure. FIG. 7A-FIG. 7B contain bar graphs showing log transformed RT-qPCR analysis confirming plasmid overexpression for experiments in (FIG. 7A) HME1 and (FIG. 7B) MCF10a cells. Data are presented as normalized mean±SEM.

FIG. 8A-FIG. 8C is an exemplary embodiment showing full length BCAR4 alters cell cycle and proliferation in accordance with the present disclosure. FIG. 8A contains representative dot plots of EdU-DNA stain (FxCycle) flow cytometry analysis and quantification of G1, S, G2/M cell populations after overexpressing empty vector (EV) or full length BCAR4 in HME1 cells. Data presented as normalized mean±SEM. Paired ratiometric two-tailed t-tests were performed. FIG. 8B shows a cell growth curve analysis of overexpressing EV or full length BCAR4 in HME1 cells. Paired two-tailed t-tests were performed. FIG. 8C shows log transformed RT-qPCR analysis from cells overexpressing EV or full length BCAR4. Data are presented as normalized mean±SEM, n=4. *p<0.05, **p<0.01

FIG. 9 is an exemplary embodiment showing BCAR4 ORF structure and domain prediction in accordance with the present disclosure. FIG. 9 is a schematic showing the predicted secondary structure of BCAR4 ORF contains 44% helices, 16% strand, and 41% coil residues.

FIG. 10A-FIG. 10B is an exemplary embodiment showing BCAR4 gene fusions alter cell-cycle and proliferation in accordance with the present disclosure. FIG. 10A and FIG. 10B contain representative dot plots of EdU-DNA stain (FxCycle) flow cytometry analysis and quantification of G1, S, G2-M cell populations overexpressing EV or gene fusion (L-B or Z-B fusion) in HME1 (n=4; FIG. 10A) or MCF10a cells (n=6; FIG. 10B). Bar graphs present normalized mean±SEM. Paired ratiometric two-tailed t tests were performed. FIG. 10A and FIG. 10B also contain cell growth curve analysis of HME1 (n=5) or MCF10a (n=6) cells overexpressing EV, L-B, or Z-B fusions. Graphs present mean±SEM. Paired two-tailed t tests were performed. *, P<0.05; **, P<0.01; ***, P<0.001.

FIG. 11 is an exemplary embodiment showing BCAR4 gene fusions do not alter cell viability in accordance with the present disclosure. FIG. 11 contains a bar graph quantifying cell death via annexin staining in HME1 cells overexpressing empty vector (EV) or gene fusions (L-B or Z-B). No statistical significance as determined by paired two-tailed t-test. RT-qPCR analysis confirmed overexpression of BCAR4 fusions. Bar graphs present normalized mean±SEM. n=3

FIG. 12 is an exemplary embodiment showing BCAR4 ORF structure and domain prediction in accordance with the present disclosure. FIG. 12 is a schematic showing the 7 predicted domains comprising SEQ ID NO: 3.

FIG. 13A-FIG. 13C is an exemplary embodiment showing BCAR4 ORF produces a protein in accordance with the present disclosure. FIG. 13A and FIG. 13B contain tandem mass spectrometry spectra of detected BCAR4 peptides (FIG. 13A) MYQPIQTYPWMNLSR (SEQ ID NO: 1) and (FIG. 13B) KSGSLQGTTEPSMTHSIIASTS (SEQ ID NO: 2) found in cancer patient data. y-fragments (blue) and b-fragments (red). Detailed mass spectrometry information in TABLE 1. FIG. 13C shows an aggregate Ribo-seq signal from available data in the GWIPs-vis platform showing signal across the BCAR4 transcript.

FIG. 14 is an exemplary embodiment showing BCAR4 encodes a small protein that alters cell-cycle and proliferation in accordance with the present disclosure. FIG. 14 contains a schematic depicting the BCAR4 gene, its predicted open reading frame in exon 4, and the tryptic peptides of BCAR4 detected by mass spectrometry. FIG. 14 also contains bar graphs showing BCAR4 peptide support in lung adenocarcinoma (LUAD), uterine corpus endometrial carcinoma (UCEC), ovarian serous cystadenocarcinoma (OV), and breast invasive carcinoma (BRCA).

FIG. 15 contains bar graphs showing BCAR4 gene fusion transcripts localize to the cytoplasm in accordance with the present disclosure. Isolated nuclear (filled bars) and cytoplasmic (open bars) RNA from SNU308 and TUHR14TKB cells with endogenous expression of LITAF-BCAR4 (L-B fusion) and ZC3H7A-BCAR4 (Z-B fusion) genes, n=4. Expression of positive control genes were measured to demonstrate nuclear (U1 and MALAT1) and cytoplasmic (Actin) RNA localization.

FIG. 16A-FIG. 16B is an exemplary embodiment showing BCAR4 ORF Flag-tag produces a protein in accordance with the present disclosure. FIG. 16A and FIG. 16B contain bar graphs showing protein quantification of BCAR4 ORF FLAG-tag and log transformed RT-qPCR quantification in (FIG. 15A) HME1 (n=4) and (FIG. 15B) MCF10a (n=3) cells overexpressing empty vector (EV), LITAF-BCAR4 (L-B), or mutant L-B (mut L-B). Data are presented as mean±SEM.

FIG. 17A-FIG. 17C is an exemplary embodiment showing BCAR4 encodes a small protein that alters cell-cycle and proliferation in accordance with the present disclosure. FIG. 17A and FIG. 17B contain representative dot plots of EdU-DNA stain (FxCycle) flow cytometry analysis and quantification of G1, S, G2-M cell populations of HME1 (n=6, FIG. 17A) and MCF10a (n=3, FIG. 17B) overexpressing EV, L-B, or mutant L-B (mut L-B) fusions. Bar graphs present normalized mean±SEM. Paired ratiometric two-tailed t tests were performed. FIG. 17A and FIG. 17B also contain cell growth curve analysis of HME1 (n=6) or MCF10a cells (n=4) overexpressing EV, LB, or mut L-B. Graphs present mean±SEM. Paired two-tailed t tests were performed. FIG. 17C is a Western blot of FLAG-tagged BCAR4 ORF expression in HME1 and MCF10a BCAR4-fusion-overexpressing cells, n=4.

FIG. 18A-FIG. 18D is an exemplary embodiment showing BCAR4 ORF expression is sufficient to alter cell cycle and proliferation in MCF10a cells in accordance with the present disclosure. FIG. 18A contains representative dot plots of EdU-DNA stain (FxCycle) flow cytometry analysis and quantification of G1, S, G2/M cell populations after overexpressing empty vector (EV), LITAF-BCAR4 (L-B), or ORF-only (ORF) fusion in MCF10a cells. FIG. 18A also contains log transformed RT-qPCR data from cell cycle analysis. Paired ratiometric two-tailed t-tests were performed. FIG. 18B is a cell growth curve analysis of MCF10a cells overexpressing EV, L-B fusion, or the BCAR4 ORF-only. Unpaired two-tailed t-tests were performed. FIG. 18C contains a Western blot and quantification of Flag-tag protein expression in MCF10a BCAR4 ORF-only overexpressing cells. FIG. 18D is a bar graph showing log transformed RT-qPCR data from EV or BCAR4 ORF-only expressing cells for cell count and western blot experiments. Data are presented as mean±SEM. n=3. *p<0.05, **p<0.01, ***p<0.001

FIG. 19A-FIG. FIG. 19B shows uncropped protein blots from FIG. 17C and FIG. 18A-FIG. 18D. FIG. 19A is a protein blot showing HME1 samples were run on 12% gel and transferred onto a PVDF membrane. Colorimetric image of ladder is also shown. Both proteins were visualized on the same gel. Gel was re-probed for Actin. FIG. 19B is a protein blot showing 30 ug of MCF10a samples were run on 4-12% gel and transferred to a nitrocellulose membrane. Merged images of colorimetric to show protein ladder. All samples were run on the same gel, and nitrocellulose cut to probe for ACTIN and FLAG.

FIG. 20 is a schematic showing BCAR4-expressing patients found in HER2-negative subtypes in accordance with the present disclosure. The Cancer Genome Atlas (TCGA) breast cancer patient cohort has ˜10% overexpression (full length BCAR4 or gene fusion alterations) mostly in Luminal A and Luminal B subtypes.

FIG. 21 is a survival curve showing BCAR4-expressing patients (red) have worse outcome in breast cancer in accordance with the present disclosure.

FIG. 22A-FIG. 22C is an exemplary embodiment showing BCAR4 expression predicts patient survival outcome by subtype in accordance with the present disclosure. FIG. 22A-FIG. 22C contain survival curves showing (FIG. 22A) Luminal A and (FIG. 22B) Luminal B subtype breast cancer patients expressing BCAR4 (red) have significantly worse overall survival relative to their respective subtype not expressing BCAR4. (FIG. 22C) Luminal A/B subtype expressing BCAR4 (blue) have survival outcomes similar to the aggressive HER2-positive subtype.

FIG. 23 is a survival curve showing BCAR4-expressing patients (red) have worse outcome across cancer types in accordance with the present disclosure.

FIG. 24 is a bar graph showing BCAR4 expression sensitizes cells to HER2-directed treatment in accordance with the present disclosure. ZR751 cells overexpressing BCAR4 full length or gene fusions show decreased cell viability with increased lapatinib (HER2 inhibitor) treatment compared to control cells without BCAR4 expression.

FIG. 25 is a survival curve showing BCAR4-positive patients (blue) have worse outcome when treated with tamoxifen in accordance with the present disclosure.

FIG. 26 is a bar graph showing ER-positive patients with BCAR4 gene fusions have BCAR4 outlier expression and are resistant to aromatase inhibitors (ER inhibition) in accordance with the present disclosure.

FIG. 27 contains images showing BCAR4 RNA detection in breast cancer tissue in accordance with the present disclosure. Top, negative control staining with no expression of BCAR4. Bottom, custom BCAR4 probes (blue) and Erbb2/HER2 probes (red) show RNA expression in breast cancer tissue.

FIG. 28 is a schematic showing BCAR4-positive patients are a distinct subgroup of the Luminal subtype responding differently to current therapies in accordance with the present disclosure.

FIG. 29A-FIG. 29D is an exemplary embodiment showing BCAR4 gene fusions increase proliferation and in activate HER3 signaling in breast cancer in accordance with the present disclosure. FIG. 29A and FIG. 29B show EdU cell proliferation flow cytometry analysis in full-length BCAR4 and LITAF-BCAR4 overexpressed ZB-75-1 breast cancer cell lines, n=3. FIG. 29C shows expression of constructs is confirmed by FLAG western blot. FIG. 29D shows protein expression analysis of ZR-75-1 cells were treated with 500 uM lapatinib. Data are presented as normalized mean+SEM, paired ratiometric two-tailed t-test were performed. *p<0.05, **p<0.01

FIG. 30 contains graphs showing high levels of 5-Hydroxymethyclcytosine (5hmC) correlate to expression levels of BCAR4 and ERBB2 (HER2) genes in cell lines in accordance with the present disclosure. Correlation analysis of 5hmC signal score (x-axis) at the promoter to expression (y-axis) in T47D (BCAR4+/HER2 nonamplified), LoVo (colon cancer cell line), and MNK7 (BCAR4 gene fusion, HER2 amplified) cell lines.

FIG. 31 is a survival curve showing elevated BCAR4 expression corresponds to worse overall survival in breast cancer in TCGA cohort in accordance with the present disclosure. 1,091 patients total, with each group having ˜210 patients.

FIG. 32 is a scatter plot showing the lack of relationship between HER2 and BCAR4 expression in accordance with the present disclosure.

FIG. 33 contains survival curves showing BCAR4-expressing Luminal B (PAM50) patients have worse overall survival (OS) and progression-free survival (PFS) in the TCGA cohort in accordance with the present disclosure. BCAR4+ is categorized as the top 20% and BCAR4− as the bottom 80%.

FIG. 34 contains survival curves showing BCAR4-expressing basal (PAM50) patients trend towards worse OS and PFS in TCGA cohort in accordance with the present disclosure.

FIG. 35 contains survival curves showing BCAR4-high patients that received tamoxifen only treatment had worse outcomes in TCGA cohort in accordance with the present disclosure.

FIG. 36 contains a survival curve showing BCAR4-high patients had worse distant metastasis free survival with tamoxifen treatment in GSE6532 cohort in accordance with the present disclosure. BCAR4+ is categorized as the top 20% and BCAR4− as the bottom 80%.

FIG. 37 is a graph showing BCAR4 gene fusion-expressing cells are resistance to Tamoxifen treatment in breast cancer in accordance with the present disclosure. MCF7 (ER+/HER2-negative) overexpressing cell lines expressing full length BCAR4 (B4) or LITAF-BCAR4 gene fusion (LB) were used.

FIG. 38 is a survival curve showing BCAR4-high patients that received Aromatase Inhibitor (AI) therapy associate with worse outcome in TCGA cohort in accordance with the present disclosure. BCAR4+ is categorized as the top 25% and BCAR4− as the bottom 25%.

FIG. 39 is a survival curve showing patients expressing BCAR4 are resistant to aromatase inhibitor (AI) therapy in accordance with the present disclosure.

FIG. 40 is a Western blot showing BCAR4 protein localizes to the plasma membrane breast cancer cells in accordance with the present disclosure. Positive controls: nuclear (Lamin), cytoplasm (Actin), plasma membrane (EGFR).

FIG. 41 is a graph showing BCAR4 directly interacts with HER2 (Erbb2) in accordance with the present disclosure.

FIG. 42 is a graph showing cells respond to lapatinib (HER2 inhibitor) in the presence of BCAR4 protein in accordance with the present disclosure.

FIG. 43 is a graph showing BCAR4-expressing cells respond to Herceptin (trastuzumab) in accordance with the present disclosure.

### DETAILED DESCRIPTION

The present disclosure is based, at least in part, on the discovery that the most prevalent gene fusion among multiple cancer types activates BCAR4. As shown herein, BCAR4 gene fusions may be used as a predictive biomarker and therapeutic target across cancer types.

Frequent somatic aberration in cancer genomes are chromosomal rearrangements joining active regulatory regions to oncogenes to generate activated gene fusions. Gene fusions are used as diagnostic markers, prognostic indicators, and therapeutic targets. A pan cancer analysis of 9,638 patients across 32 cancer types discovered the most prevalent gene fusions activated Breast Cancer Anti-Estrogen Resistant 4 (BCAR4), a known oncogene (see e.g., Example 1). The data indicate that BCAR4-fusions: (i) are predominately expressed in HER2 non-amplified breast cancer patients; (ii) transform benign cells; (iii) activate HER2/HER3 signaling; and (iv) sensitize cells to lapatinib (HER2 inhibitor). BCAR4 gene fusions may be important in breast cancer progression by activating HER2/HER3 signaling in HER2-negative subtypes. BCAR4 may be clinically relevant to identify patients previously excluded from HER2-targeted therapies. Additionally, full length BCAR4, and gene fusions, are relevant in patient hormone treatment resistance (see e.g., Example 2). Biomarkers of aromatase inhibitor (hormone therapy) resistance have been identified by performing transcriptome analysis of pretreatment tumor biopsies accrued from patients enrolled in the preoperative letrozole phase 2 study (NCT00084396) and the American College of Surgeons Oncology Group (ACOSOG) Z1031 study (NCT00265759). It was discovered that recurrent BCAR4 gene fusions drive outlier expression of BCAR4 in four patients (11%) that were resistant to AI therapy. Initial studies also suggest that BCAR4 may interact directly with estrogen receptor providing another avenue for therapeutic potential.

Described herein is establishing BCAR4 as a predictive biomarker to non-invasively identify patients—beyond the existing HER2 amplified population—that would benefit from HER2-targeted treatments through development of a novel diagnostic assay. Cell-free DNA is leveraged to monitor BCAR4 gene activation through an epigenetic surrogate (5hmC) of gene expression (see e.g., Example 3). The data shows the ability to monitor BCAR4 gene activation (through 5hmC marks) alongside other breast cancer biomarkers useful for subtyping. Using cell lines with varied expression of BCAR4 and ERBB2 (HER2), a positive correlation between 5hmC and gene expression was determined. Also described herein is monitoring BCAR4 expression through tissue using techniques such as quantitative RT-PCR (qPCR) and RNA in situ hybridization (RNA ISH). This gene has broader impact in other aggressive cancers (ovarian and stomach) given the prevalence of BCAR4− fusions across 11 solid tumors.

The HER2-positive (HER2+) breast cancer treatment landscape continues to rapidly expand with new effective therapies—but approved for 15% of the breast cancer population. Current clinical HER2+ patient subtyping is based on gene amplification and protein expression through tissue staining. Without gene amplification, subtyping is scored by protein staining intensity with only the most intense (highest score) subtyped as HER2+. It is this non-amplified, HER2 low score range—with intact signaling—that may be BCAR4-positive and may respond to HER2-targeted therapies. Currently there is no method to identify these patients. To address this unmet clinical need, a noninvasive liquid assay may provide a more sensitive, less invasive method to detect molecular events to improve current clinical diagnosis and inform on patient treatment. This would expand the identification of patients that would immediately benefit from the use of existing drugs. Further, the mechanistic research may offer future strategies for improved therapeutic targeting of HER2-lo/BCAR4− positive patients.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gin by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Cancer Treatments

Described herein are methods of treating a subject having or suspected of having a HER2 non-amplified (HER2-negative) cancer or low HER2 expressing (HER2-low) cancer. The methods herein may be used to determine the appropriate cancer treatment for a subject, in particular whether a subject would benefit from or respond to a HER2-targeted treatment.

HER2-Targeted Cancer Treatments

As used herein, a HER2-targeted cancer treatment refers to a class of medicines traditionally used to treat HER2-positive cancers.

The HER2-positive treatment landscape historically, and recently, continues to expand with new, exciting therapies improving patient outcome. There are at least 9 HER2-directed FDA-approved therapies to treat HER2-positive breast cancer; three of these therapies were approved in the last two years. However, these therapies are only approved for the 15% of breast cancer patients clinically diagnosed as HER2-positive. Current clinical HER2-positive patient subtyping is based on gene amplification and protein expression through tissue staining. Without gene amplification, subtyping is scored by protein staining intensity with only the most intense (highest score) subtyped as HER2-positive. As described herein, it was surprisingly found that patients having HER2 non-amplified (HER2-negative) cancer or low HER2 expressing (HER2-low) cancer may also benefit from HER2-targeted cancer treatments.

In some embodiments, a HER2-targeted cancer treatment comprises a monoclonal antibody against HER2. For example, the monoclonal antibody can be Herceptin (trastuzumab), Perjeta (pertuzumab), Hylecta, Herzuma, Kanjinti, Ogivri, Ontruzant, Trazimera, or Margenza (margetuximab-cmkb).

In some embodiments, a HER2-targeted cancer treatment comprises an antibody-drug conjugate targeted to HER2. For example, the antibody-drug conjugate can be Enhertu (am-trastuzumab-deruxtecan-nxki), Kadcyla (T-DM1 or ado-trastuzumab emtansine), or Phesgo (pertuzumab, trastuzumab, and hyaluronidase-zzxf).

In some embodiments, a HER2-targeted cancer treatment comprises a small molecule inhibitor of HER2. For example, the small molecule inhibitor can be Nerlynx (neratinib), Tykerb (lapatinib), Tukysa (tucatinib), pyrotinib, or afatinib.

As another example, a HER2-targeted cancer treatment comprises a short hairpin RNA (shRNA) or a short interfering RNA (siRNA) targeting HER2.

As another example, a HER2-targeted cancer treatment comprises a single guide RNA (sgRNA) targeting HER2.

In some embodiments, a subject may be administered a cancer treatment that is not HER2-targeted, e.g., a cancer treatment that is not specifically indicated for HER2 positive cancer. For example, a subject may be administered a cancer treatment that is not HER2-targeted if the subject is predicted to be unresponsive to a HER2-targeted cancer treatment using the methods of the present disclosure. As another example, a subject that is predicted to be responsive to a HER2-targeted cancer treatment may be administered a cancer treatment that is not HER2-targeted in combination with or in addition to a HER2-targeted cancer treatment.

A cancer treatment that is not HER2-targeted may be any such treatment known in the art. For example, a cancer treatment that is not HER2-targeted may comprise hormone therapy, chemotherapy, immunotherapy, or radiotherapy.

Hormone Therapy

Hormone therapy is a type of cancer treatment that slows or stops the growth of cancers that use hormones to grow. Examples of hormone therapies include aromatase inhibitors (AIs), such as anastrozole, exemestane, and letrozole; selective estrogen receptor modulators (SERMs), such as tamoxifen and raloxifene; estrogen receptor antagonists, such as fulvestrant and toremifene; luteinizing hormone-releasing hormone (LHRH) agonists, such as goserelin, leuprolide, and triptorelin; anti-androgens, such as apalutamide, enzalutamide, darolutamide, bicalutamide, flutamide, and nilutamide (also called androgen deprivation therapy or ADT); CYP17 inhibitors, such as abiraterone and ketoconazole; luteinizing hormone-releasing hormone (LHRH) agonists and antagonists, such as goserelin, leuprolide, triptorelin, and degarelix; progestins, such as medroxyprogesterone acetate or megestrol acetate; or adrenolytics, such as mitotane.

Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents can include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines such as altretamine, triethylenemelamine, trietylenephosphoramide, triethiylenethiophosphoramide and trimethylolomelamine; acetogenins (such as bullatacin and bullatacinone); a camptothecin (such as the synthetic analogue topotecan); bryostatin; callystatin; CC-1065 (such as its adozelesin, carzelesin and bizelesin synthetic analogues); cryptophycins (particularly cryptophycin 1 and cryptophycin 8); dolastatin; duocarmycin (such as the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, such as dynemicin A; uncialamycin and derivatives thereof; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-l-norleucine, doxorubicin (e.g., morpholino-doxorubicin, cyanomorpholino-doxorubicin, 2-pyrrolino-doxorubicin, or deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, or methotrexate or pharmaceutically acceptable salts, acids or derivatives of any of the above. Other examples of chemotherapeutic agents can be Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Emtansine, Adriamycin (Doxorubicin Hydrochloride), Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran (Melphalan Hydrochloride), Alkeran (Melphalan), Alimta (Pemetrexed Disodium), Aloxi (Palonosetron Hydrochloride), Ambochlorin/Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar (Irinotecan Hydrochloride), Capecitabine, CAPOX, Carac (Fluorouracil-Topical), Carboplatin, Carboplatin-Taxol, Carfilzomib, Carmubris (Carmustine), Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, Chlorambucil-prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-bevacizumab, FOLFIRI-Cetuximab, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine—Oxaliplatin, Gemtuzumab Ozogamicin, Gemzar (Gemcitabine Hydrochloride), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Keoxifene (Raloxifene Hydrochloride), Kepivance (Palifermin), Keytruda (Pembrolizumab), Kisqali (Ribociclib), Kyprolis (Carfilzomib), Lanreotide Acetate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituximab, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Treanda (Bendamustine Hydrochloride), Trifluridine and Tipiracil Hydrochloride, Trisenox (Arsenic Trioxide), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate) or pharmaceutically acceptable salts, acids or derivatives of any of the above.

Immunotherapy In the context of cancer treatment, immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., CAR T, mesenchymal stem cells) or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of bispecific monoclonal antibody (BsMAb) therapy. For example, the BsMAb therapy can be any one or more of the currently FDA-approved BsMAb therapies, such as blinatumomab, emicizumab, or amivantamab. In one aspect of immunotherapy, the tumor cell must bear some marker that is amenable to targeting, i.e., is not present on the majority of other cells. Many tumor markers exist and any of these may be suitable for targeting in the context of the present disclosure. Other common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8, and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

In some embodiments, the immunotherapy in accordance with the present disclosure is CAR T cell therapy (e.g., CD19-specific chimeric antigen receptor T (CAR-T)). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

Checkpoint Immunotherapy

In some embodiments, a checkpoint immunotherapy may be administered as a cancer treatment. An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer do not work directly on the tumor at all. They only take the brakes off an immune response that has begun but has not yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), Atezolizumab, or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter can repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer or tumor. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment, the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity-modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

Therapeutic Methods

Also provided is a process of treating, preventing, or reversing cancer in a subject in need of administration of a therapeutically effective amount of a HER2-targeted cancer treatment, so as to inhibit cancer cell proliferation and cell cycle progression.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a HER2-targeted cancer treatment is, for example, an amount that would cause the desired therapeutic effect in a subject while minimizing undesired side effects. In various embodiments, an effective amount of a HER2-targeted cancer treatment described herein can substantially inhibit cancer cell proliferation and cell cycle progression, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of HER2-targeted cancer treatment can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit cancer cell proliferation and cell cycle progression.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a HER2-targeted cancer treatment can occur as a single event or over a time course of treatment. For example, a HER2-targeted cancer treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

A HER2-targeted cancer treatment can be administered simultaneously or sequentially with another agent, such as an antibiotic, an anti-inflammatory, or another agent. For example, a HER2-targeted cancer treatment can be administered simultaneously with another agent, such as an antibiotic or an anti-inflammatory. Simultaneous administration can occur through administration of separate compositions, each containing one or more of a HER2-targeted cancer treatment, an antibiotic, an anti-inflammatory, or another agent. Simultaneous administration can occur through administration of one composition containing two or more of a HER2-targeted cancer treatment, an antibiotic, an anti-inflammatory, or another agent. A HER2-targeted cancer treatment can be administered sequentially with an antibiotic, an anti-inflammatory, or another agent. For example, a HER2-targeted cancer treatment can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the HER2-targeted cancer treatment may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a HER2-targeted cancer treatment such as a compound described herein (e.g., lapatinib) may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to HER2-targeted cancer treatment or HER2 inhibitor, biological samples, sequencing reagents, cell-free DNA, etc. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Pan-Cancer Analysis Reveals Recurrent BCAR4 Gene Fusions Across Solid Tumors

This example describes discovery of a functionally recurrent class of BCAR4 gene fusions that act through a protein to alter cell cycle and proliferation across solid tumors.

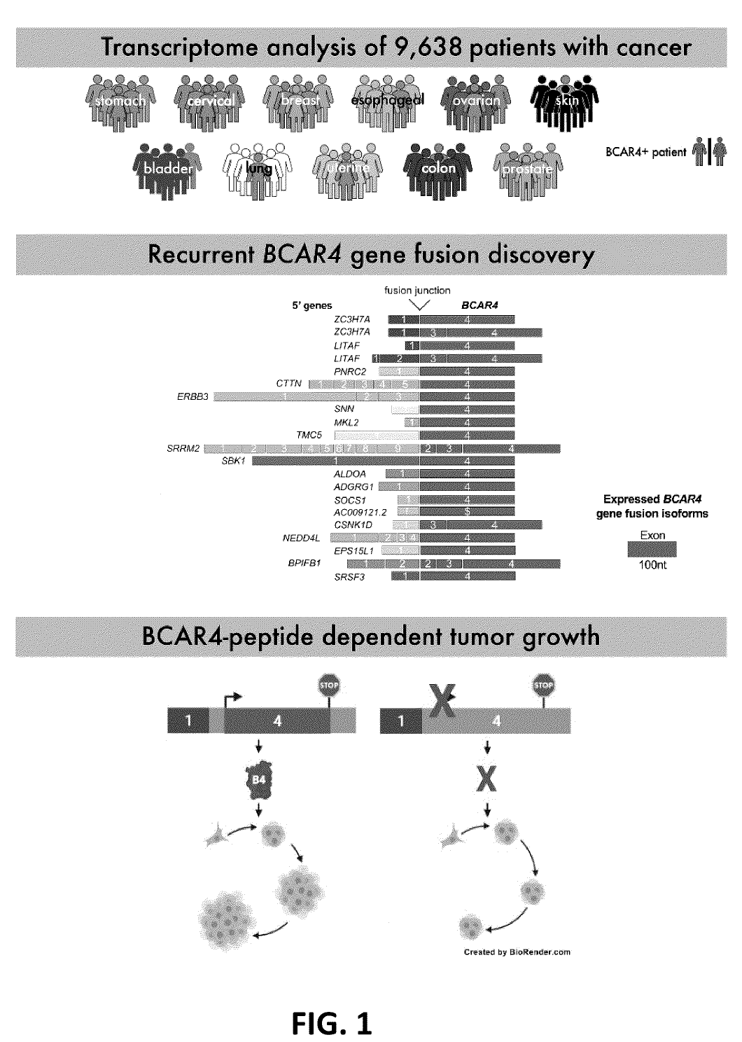

Chromosomal rearrangements often result in active regulatory regions juxtaposed upstream of an oncogene to generate an expressed gene fusion. Repeated activation of a common downstream partner—with differing upstream regions across a patient cohort-suggests a conserved oncogenic role. Analysis of 9,638 patients across 32 solid tumor types revealed an annotated long noncoding RNA (lncRNA), Breast Cancer Anti-Estrogen Resistance 4 (BCAR4), was the most prevalent, uncharacterized, downstream gene fusion partner occurring in 11 cancers (see e.g., FIG. 1). Its oncogenic role was confirmed using multiple cell lines with endogenous BCAR4 gene fusions. Furthermore, overexpressing clinically prevalent BCAR4 gene fusions in untransformed cell lines was sufficient to induce an oncogenic phenotype. As shown herein, the minimum common region to all gene fusions harbors an open reading frame that is necessary to drive proliferation. BCAR4 gene fusions represent an underappreciated class of gene fusions that may have biological and clinical implications across solid tumors.

Introduction

Chromosomal rearrangements are common somatic aberrations in cancer genomes, often leading to the juxtaposition of two genes, creating gene fusions. In addition to their biological roles in oncogenesis, some gene fusions are clinically relevant diagnostic markers, prognostic indicators, and therapeutic targets. Advances in next-generation sequencing coupled with improved gene fusion detection tools have accelerated the discovery of novel gene fusions in solid tumors. E26 transformation-specific (ETS) transcription factor family members, ALK and ROS fusions in lung cancer, and RAF kinase gene fusions in melanoma, gastric, and prostate cancer represent important gene fusion discoveries across cancers. These exemplify a biological pattern called “functional recurrence”: various 5′ regulatory regions join a particular 3′ oncogene across samples and cancer types. While a single gene fusion event may be infrequent and overlooked, functionally recurrent fusions activating the same downstream partner can accentuate highly relevant oncogenes and provide molecular insight into their function. Some gene fusions are low frequency (<5%) events in a single cancer (even with functionally recurrent events), but accumulation across solid tumors expands the potential biological and clinical relevance to a significantly broader patient population.

In this study, INTEGRATE, a highly sensitive gene fusion discovery tool, was applied in a pan-cancer strategy to detect functionally recurrent gene fusions. From an analysis of 9,638 patients across 32 different cancer types within The Cancer Genome Atlas (TCGA) consortium, a novel class of Breast Cancer Anti-Estrogen Resistance 4 (BCAR4) gene fusions was prioritized because: (i) they are the most prevalent gene fusions not previously characterized across solid tumors; (ii) full-length BCAR4 is implicated in cancer; and (iii) all patients express a minimum common region of BCAR4 that contains an open reading frame (ORF), suggesting a conserved oncogenic role. Silencing the two most common fusions (LITAFBCAR4 and ZC3H7A-BCAR4) decreased proliferation in cancer cell lines; conversely, overexpressing the fusions in benign models increased it. Mutating the ORF abated BCAR4 gene fusion-driven proliferation. Collectively, this pan-cancer analysis discovered a functionally recurrent class of BCAR4 gene fusions that act through a protein to alter cell-cycle and proliferation across solid tumors.

Results

Pan-Cancer Analysis Discovers Recurrent BCAR4 Fusions

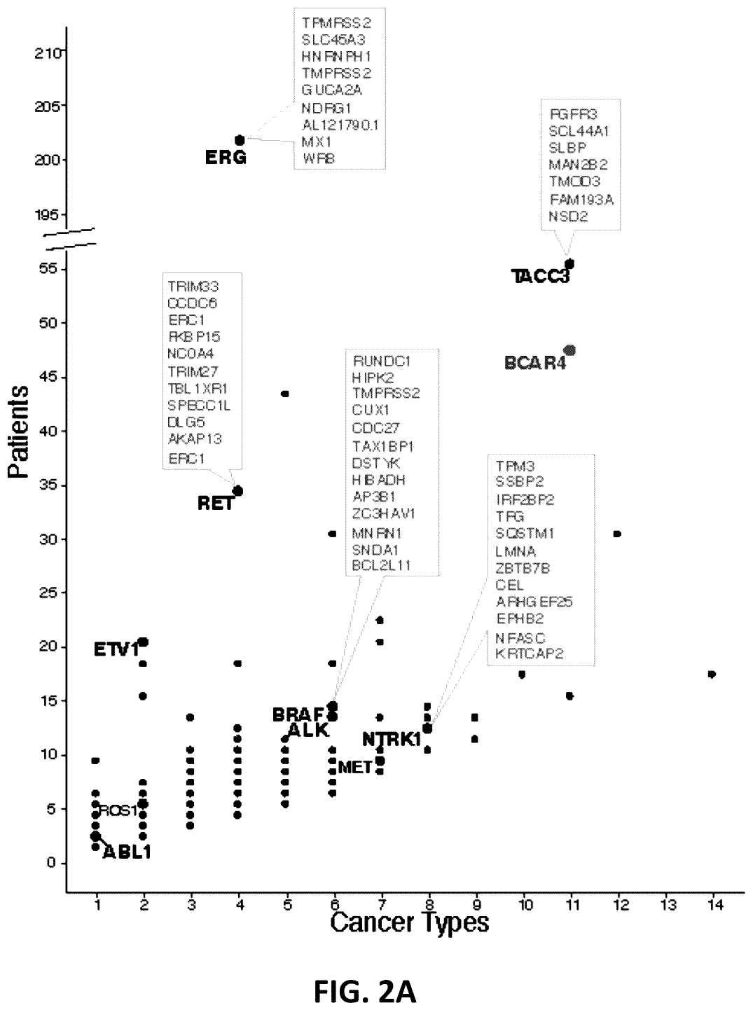

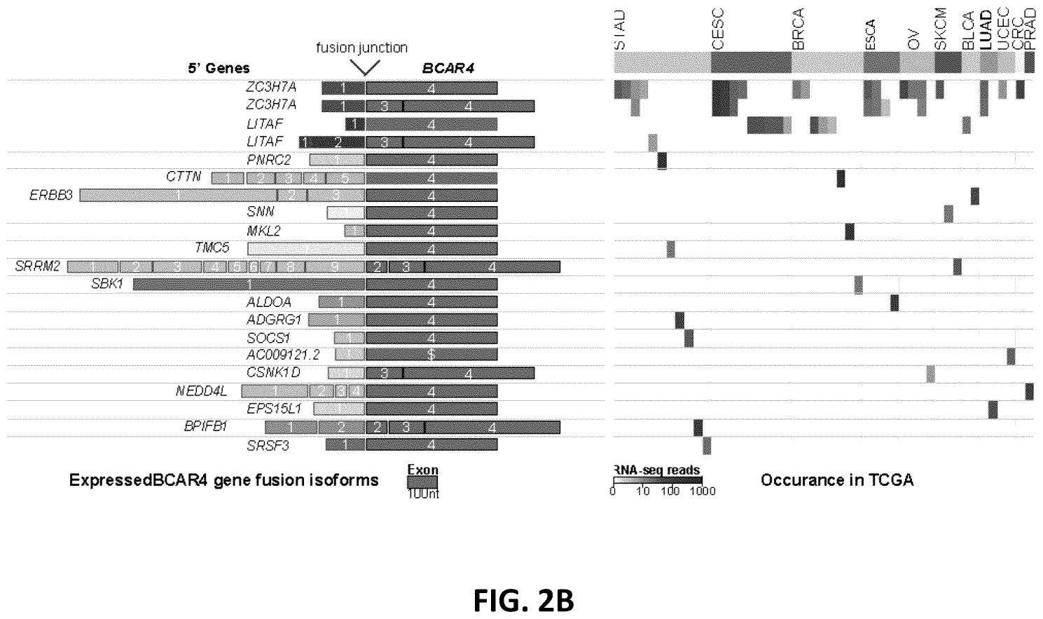

INTEGRATE was used to analyze RNA-seq data from 9,638 patients across 32 different cancer types as part of TCGA consortium. To identify functionally recurrent gene fusion candidates, genes that were recurrent 3′ gene fusion partners despite having different 5′ partners were prioritized. As shown in FIG. 2A, the two most prevalent recurrent 3′ gene fusion partners are ERG (202 patients) and TACC3 (55 patients). This is expected as ERG fusions with androgen-sensitive 5′ partners, including TMPRSS2, are highly recurrent gene fusions in prostate cancer. Further, FGFR3-TACC3 is a highly recurrent gene fusion observed across multiple solid tumors. The third most prevalent class of gene fusions resulted in the expression of an annotated lncRNA-BCAR4-in 47 patients across 11 cancer types. BCAR4 gene fusions are more prevalent than known clinically actionable recurrent gene fusions with immediate translational impact such as ABL1, BRAF, and ALK fusions.

The most common BCAR4 gene fusion events are intrachromosomal rearrangements with breakpoints between the initial untranslated exons of either ZC3H7A or LITAF and the fourth exon of BCAR4 (see e.g., FIG. 2B). This results in the regulatory regions of ZC3H7A or LITAF minimally activating the expression of the fourth exon of BCAR4. Similarly, most expressed BCAR4 gene fusion transcripts include early exons of 5′ partners spliced upstream of exon 4 of BCAR4. This functionally recurrent structure of BCAR4 gene fusions across patients suggests exon 4 is the minimum region necessary to drive its function. Importantly, BCAR4 gene fusions are tumor specific with no detection in adjacent normal tissues available from 718 TCGA patients.

Silencing BCAR4 Gene Fusions Decreases Cell-Cycle Progression and Proliferation in Cancer Cells

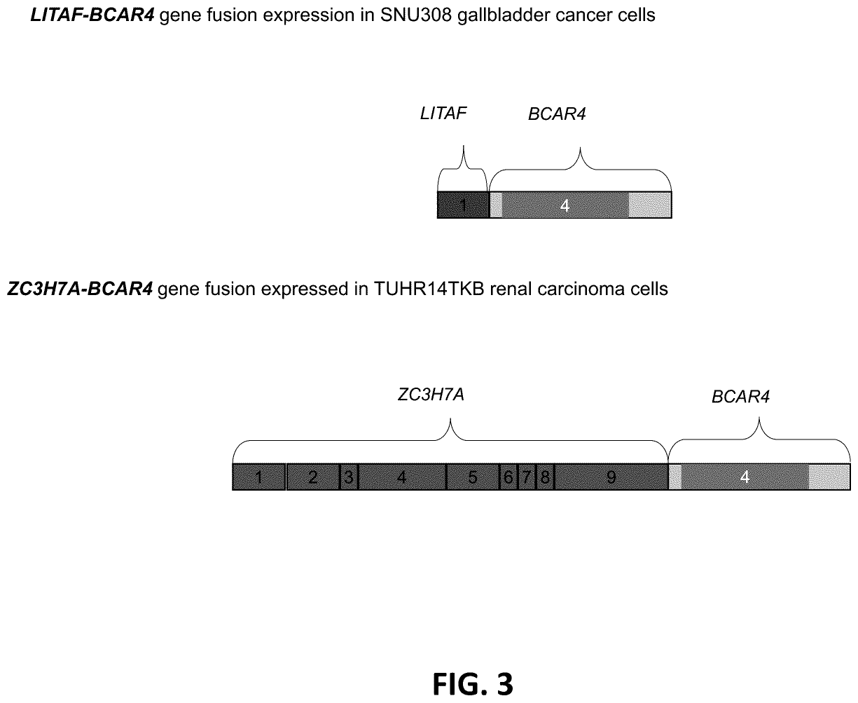

BCAR4 is a known oncogene promoting tumor growth, suggesting a cancer relevance for BCAR4 gene fusions. To identify cancer cell lines harboring BCAR4 fusions, RNA-seq data from the Cancer Cell Line Encyclopedia (CCLE) was analyzed using INTEGRATE and two cell lines harboring the most common 5′ gene fusion partners were found: SNU308 gallbladder cancer cells (expressing LITAF-BCAR4) and TUHR14TKB renal carcinoma cells (expressing ZC3H7A-BCAR4; see e.g., FIG. 3). These patient-derived lines, endogenously expressing only BCAR4 fusions, are ideally suited to study the effects of BCAR4 fusions on cancerous phenotypes.

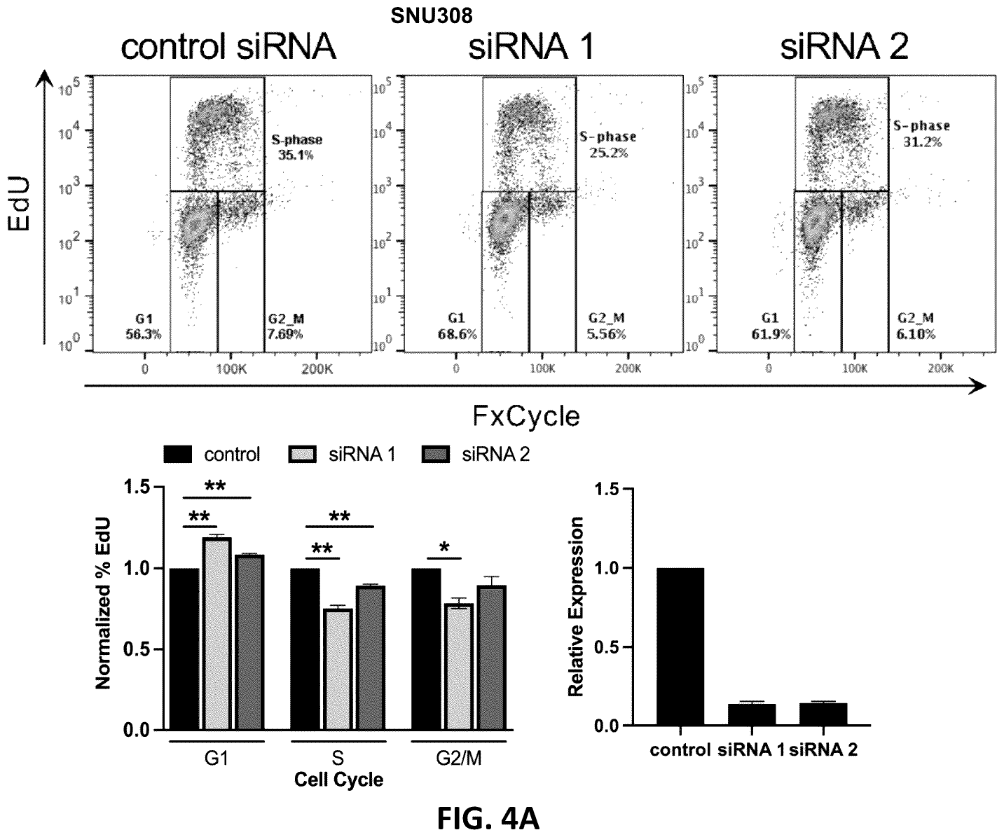

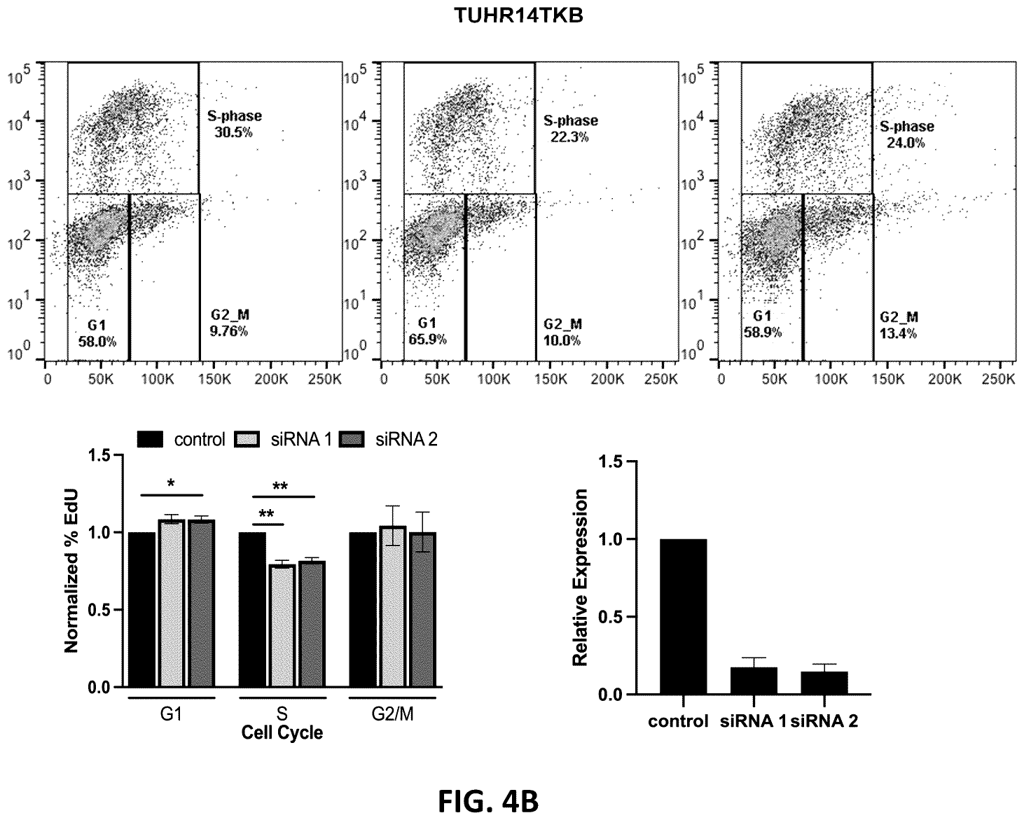

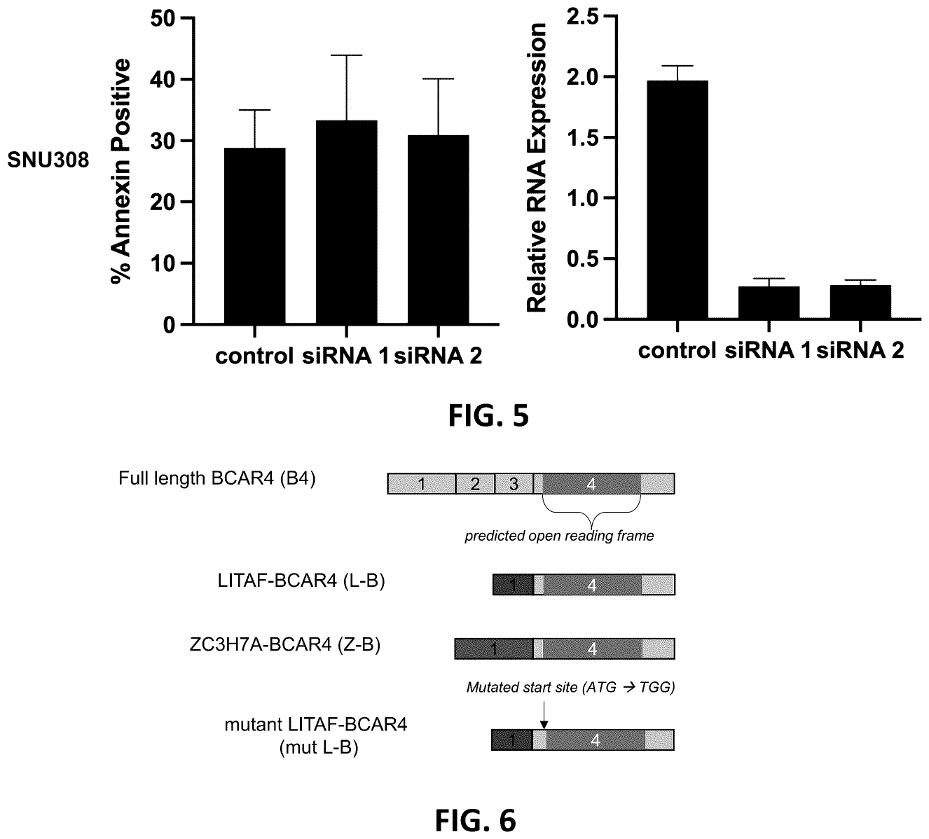

To determine whether the BCAR4 fusions drive cell-cycle progression in cancer cells, the fusions were transiently silenced with siRNAs and the cell-cycle profile assessed with flow cytometry to monitor EdU incorporation and DNA content. Knockdown of fusion expression resulted in significantly fewer S-phase cells and increase in G1 phase cells (see e.g., FIG. 4A and FIG. 4B). Indeed, these fusion-targeting siRNAs consistently reduced the proportion of S-phase cells by 10% to 25% across these cell lines and increased the proportion of G1 phase cells by 5% to 19%. Knockdown of BCAR4 gene fusions did not alter cell viability as determined by annexin staining (see e.g., FIG. 5). These data show that endogenously expressed BCAR4 gene fusions alter cell-cycle, specifically S-phase entry, in two different cancer cell lines.

BCAR4 Gene Fusions Increase Cell-Cycle Progression and Proliferation in Benign Cells

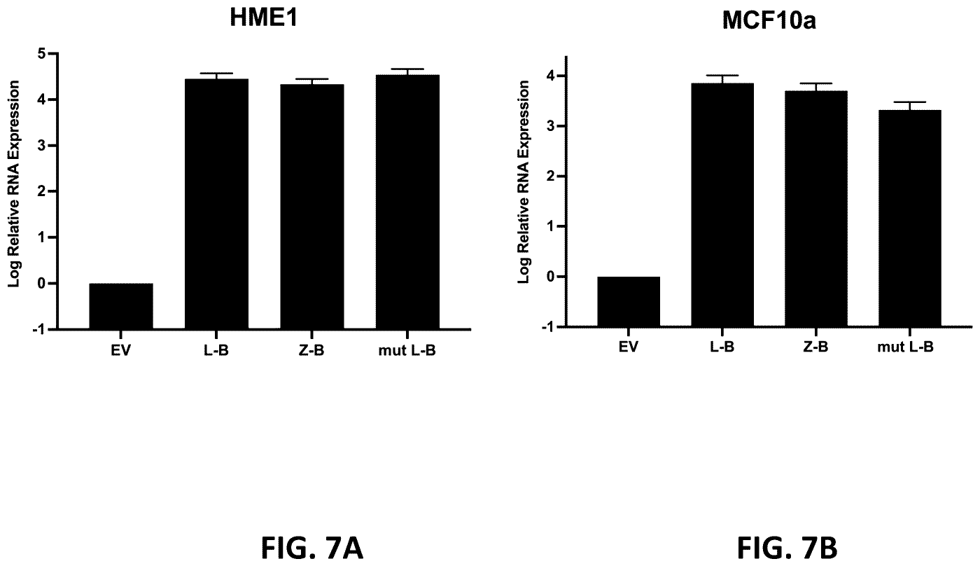

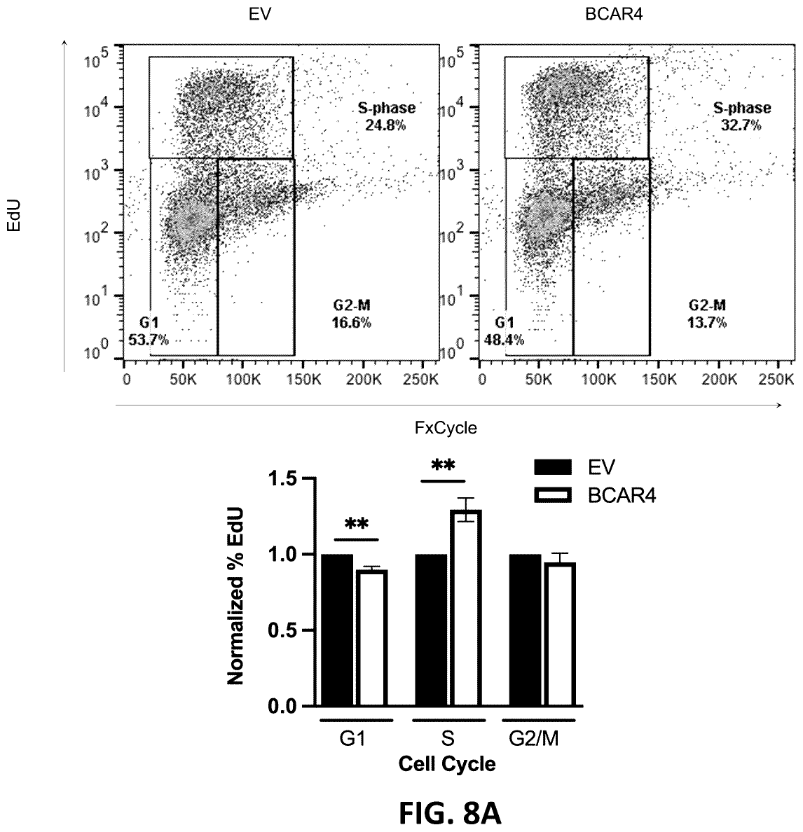

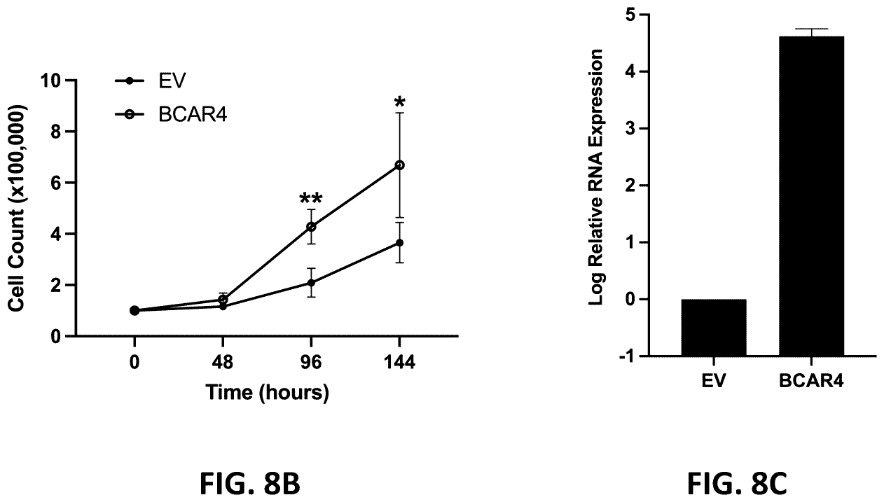

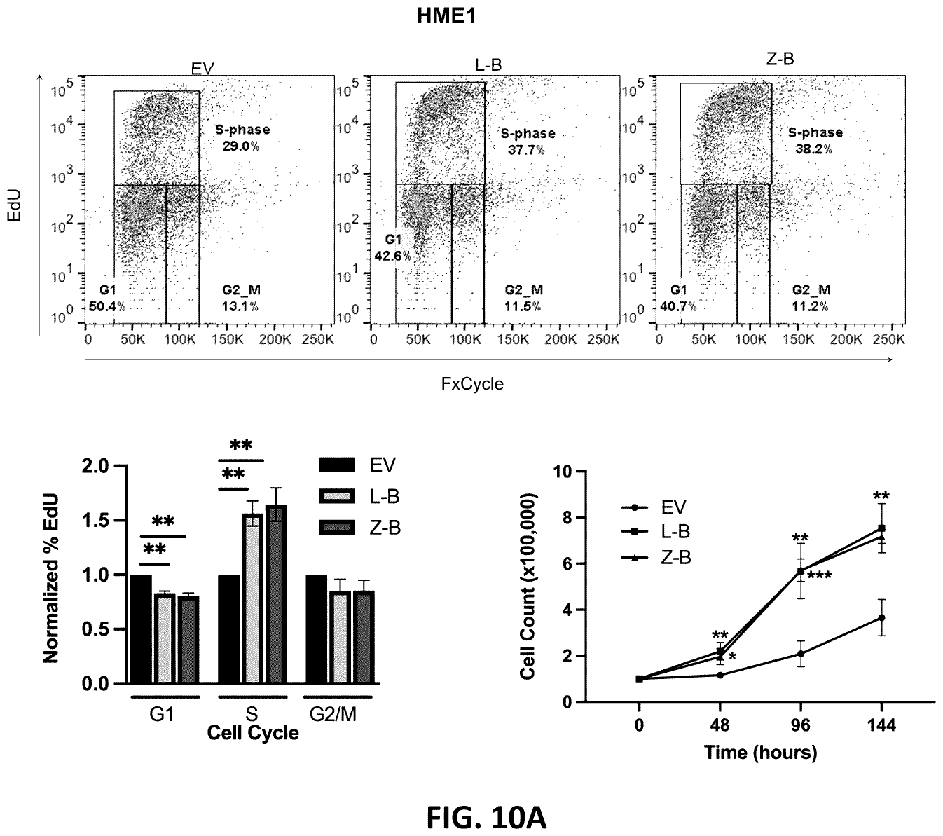

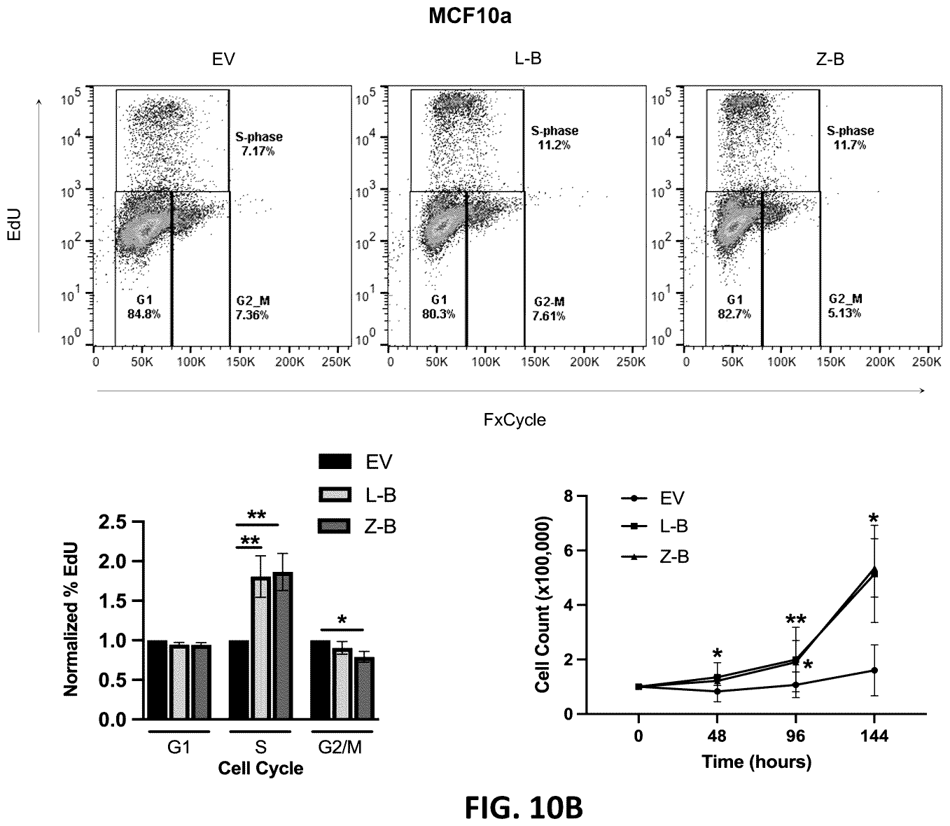

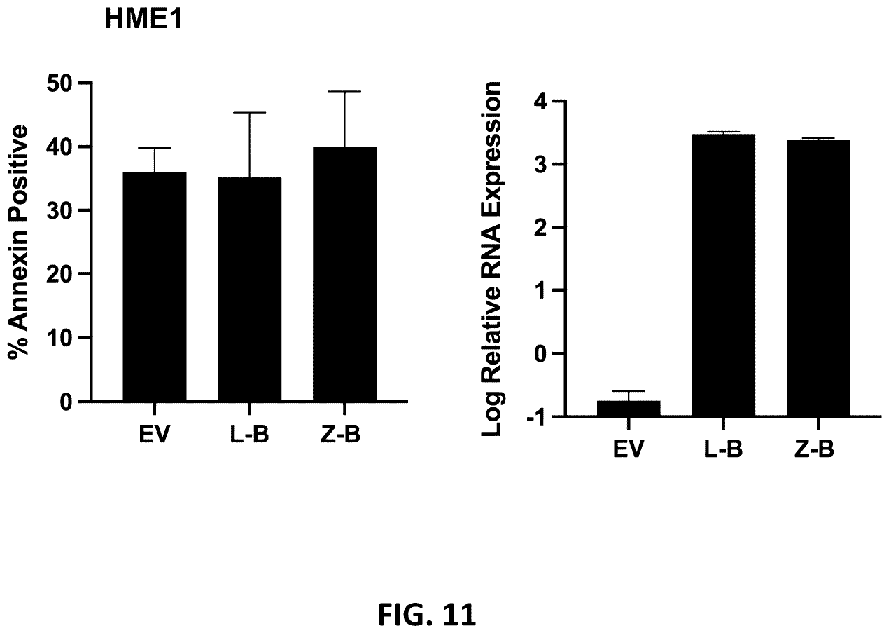

The data show that endogenous BCAR4 fusions influence the behavior of cancer cells; next, whether BCAR4 gene fusions can drive proliferation in normal epithelial cell lines was evaluated. It was hypothesized that expression of full length BCAR4 or the common BCAR4 fusions would drive cell-cycle progression resulting in more proliferating (S-phase) cells and fewer G1-phase cells. Full length BCAR4, the LITAF-BCAR4 (L-B fusion), or the ZC3H7A-BCAR4 fusion (Z-B fusion) were introduced into untransformed cell lines (HME1 and MCF10a; see e.g., FIG. 6) and expression levels were monitored by qRT-PCR (FIG. 7A-FIG. 7B); the cell-cycle profile and proliferation were then assessed. Cell-cycle analysis confirmed that HME1 cells expressing full length BCAR4 had 29% more S-phase cells than empty vector (EV) controls (see e.g., FIG. 8A-FIG. 8C). Interestingly, the L-B and Z-B fusions more efficaciously drove HME1 cells into S-phase (56% and 65% increases, respectively; see e.g., FIG. 10A). Similarly, expression of BCAR4 gene fusions in MCF10a cells significantly increased the proportion of S-phase cells (greater than 80% relative to EV; see e.g., FIG. 10B). There were no concurrent changes in cell viability (see e.g., FIG. 11). These results demonstrate BCAR4 fusion expression can promote cell-cycle progression in benign cell lines.

To determine whether the observed increase in cell-cycle progression leads to an increase in cellular proliferation, the growth of full length BCAR4− and fusion-overexpressing cells were monitored with cell counting. Significant increases in the number of HME1 cells expressing the L-B and Z-B fusions were observed starting at 48 hours, and at 96 hours there were approximately 3-fold more cells relative to EV controls (see e.g., FIG. 10A). Expression of full length BCAR4 yielded more modest results (<2-fold more than EV at 96 hours; see e.g., FIG. 8B). MCF10a BCAR4-fusion cells also had increased cellular proliferation (see e.g., FIG. 10B). These data indicate that BCAR4 gene fusion expression is sufficient to increase cell growth in two normal cell lines.

BCAR4 Encodes a Small Peptide Responsible for the Oncogenic Phenotypes

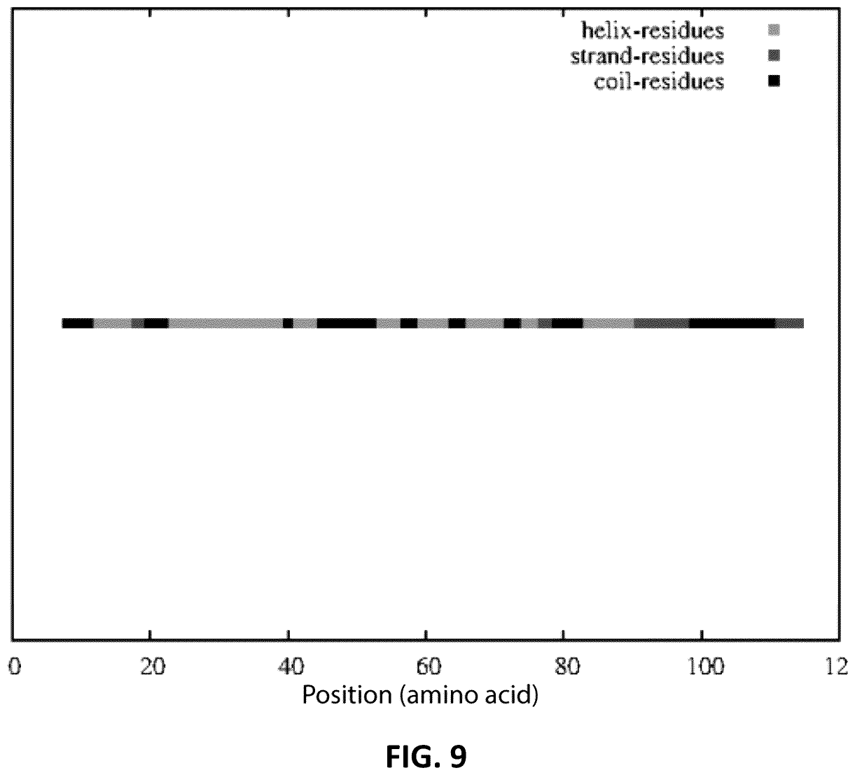

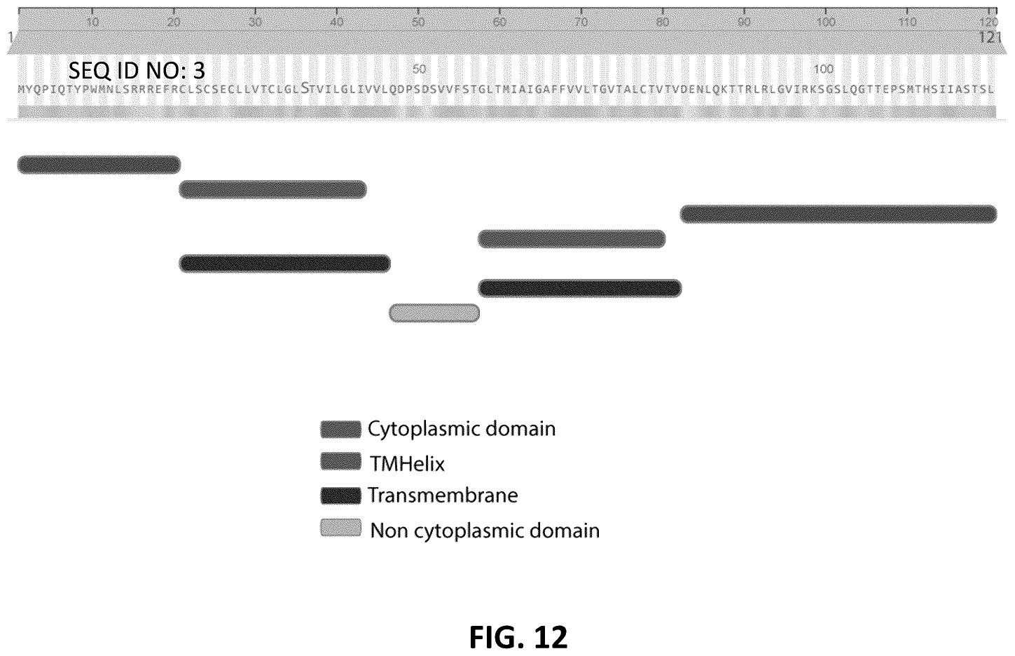

Since the fourth exon of BCAR4 is the minimum common region found in all gene fusion events across cancers, it was speculated to be functionally important. A sequence analysis of BCAR4 identified a previously reported small peptide. The BCAR4 ORF is 366 nucleotides in length and produces a 121 amino acid peptide chain predicted to have structure and multiple transmembrane domains (see e.g., FIG. 9 and FIG. 12). The 121 amino acid sequence is shown in FIG. 12 and below:

SEQ ID NO: 3:
MYQPIQTYPW MNLSRRREFR CLSCSECLLV TCLGLSTVIL
GLIVVLQDPS DSVVFSTGLT MIAIGAFFVV LTGVTALCTV
TVDENLQKTT RLRLGVIRKS GSLQGTTEPS MTHSIIASTS
L

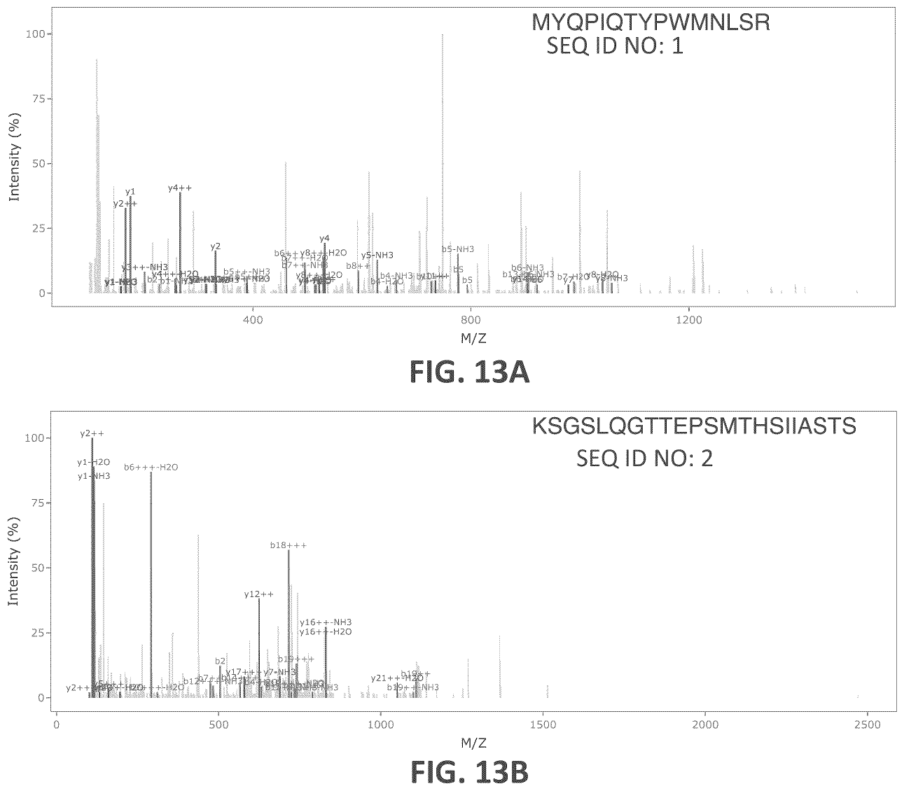

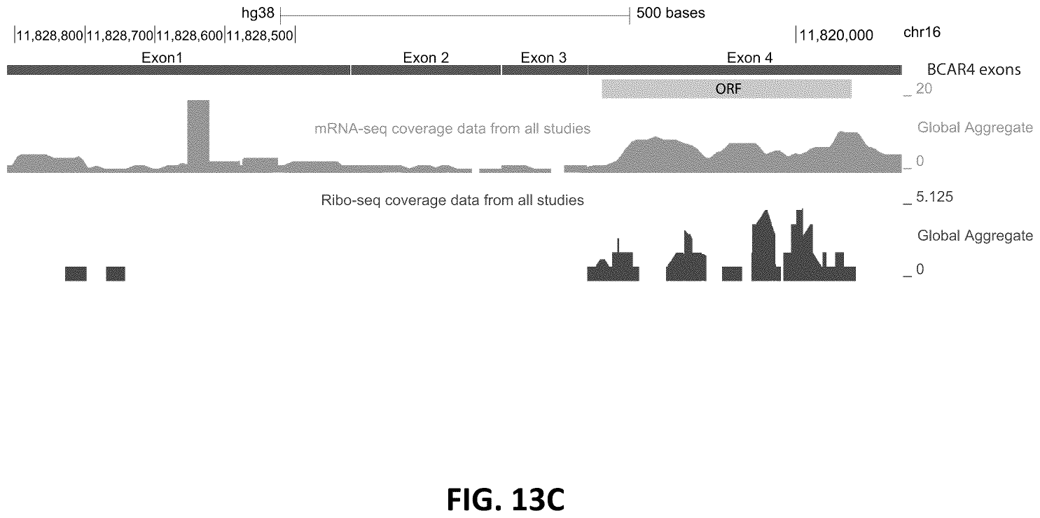

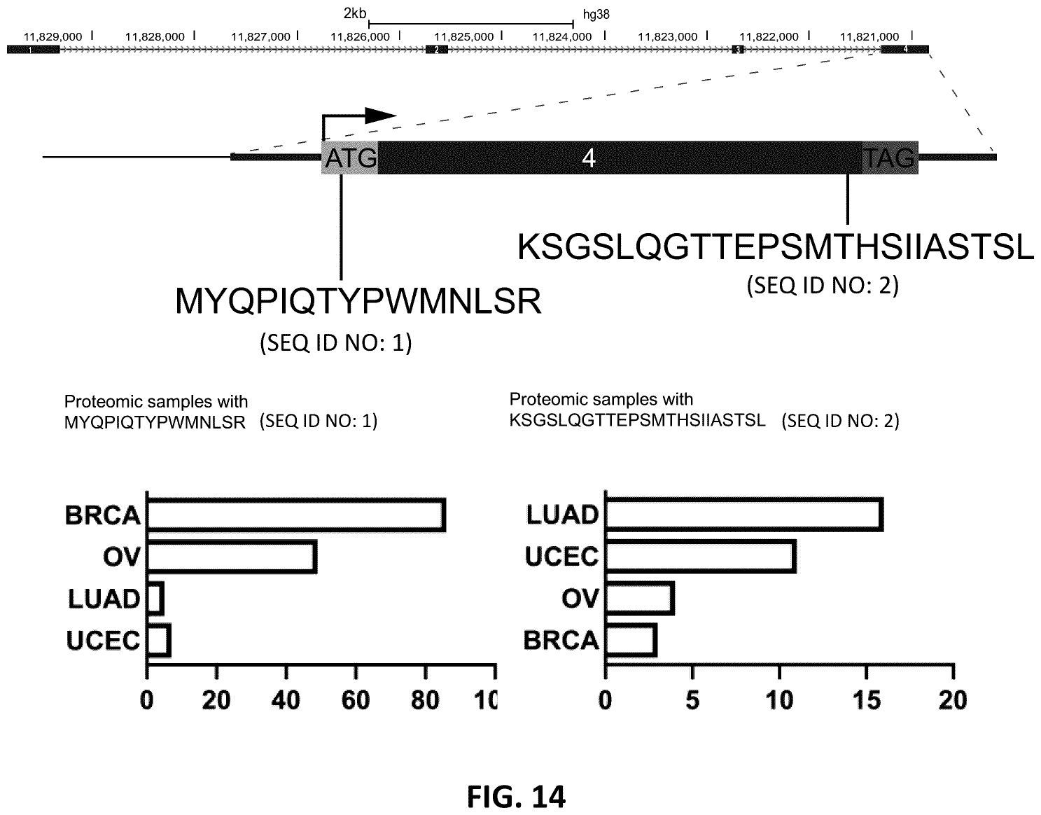

Evidence of the BCAR4 peptide in cancer patient samples was found by mining publicly available mass spectrometry data. Two different semi-tryptic digestion fragments corresponding to the BCAR4 protein were reliably detected in at least four cancer types (see e.g., FIG. 14, FIG. 13A-FIG. 13B, and TABLE 4).

TABLE 4
Peptide Spectrum Matches from PepQuery.

| | | | | | | | | | |
|---|---|---|---|---|---|---|---|---|---|
| | Spectrum | | Exp | | Pep | | | | |
| Modification | title | Charge | mass | ppm | mass | mz | Score | P val | Cancer |

Peptide: KSGSLQGTTEPSMTHSIIASTSL (SEQ ID NO: 2)

| | | | | | | | | | |
|---|---|---|---|---|---|---|---|---|---|
| TMT 10-plex | 05CPTAC_ | 13 | 2806.486044 | −2.574365002 | 2806.478819 | 936.5026245 | 14.71601972 | 0.001998002 | UCEC |
| of peptide | UCEC_W_ | | | | | | | | |
| N-term@0 | PNNL_20 | | | | | | | | |
| [229.1629]; | 170922_ | | | | | | | | |
| TMT | B2S1_ | | | | | | | | |
| 10-plex of | f17: | | | | | | | | |
| K@1 | 41692: | | | | | | | | |
| [229.1629]; | 3 | | | | | | | | |
| Oxidation | | | | | | | | | |
| ofM@13 | | | | | | | | | |
| [15.9949] | | | | | | | | | |
| TMT 10-plex | 03CPTAC_ | 3 | 2806.502707 | −8.511554886 | 2806.478819 | 936.5081787 | 14.7104528 | 0.002997003 | UCEC |
| of peptide N- | UCEC_W_ | | | | | | | | |
| term@0[229. | PNNL_20 | | | | | | | | |
| 1629];TMT | 170922_B | | | | | | | | |
| 10-plex of | 1S3_f17:4 | | | | | | | | |
| K@1[229.162 | 0235:3 | | | | | | | | |
| 9]; Oxidation | | | | | | | | | |
| of | | | | | | | | | |
| M@13[15.9949] | | | | | | | | | |
| TMT 10-plex | 02CPTAC_ | 3 | 2806.484396 | −1.987170398 | 2806.478819 | 936.5020752 | 14.68436384 | 0.002997003 | UCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 1S2_f17: | | | | | | | | |
| K@1[229.162 | 39784: | | | | | | | | |
| 9]; Oxidation | 3 | | | | | | | | |
| of | | | | | | | | | |
| M@13[15.9949] | | | | | | | | | |
| TMT 10-plex | 01CPTAC_ | 3 | 2806.483664 | −1.726195019 | 2806.478819 | 936.5018311 | 14.66059035 | 0.002997003 | UCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 9984: | | | | | | | | |
| K@1[229.162 | 31S1_f17: | | | | | | | | |
| 9]; Oxidation | 3 | | | | | | | | |
| of | | | | | | | | | |
| M@13[15.9949] | | | | | | | | | |
| TMT 10-plex | 08CPTAC_ | 3 | 2806.479635 | −0.290830431 | 2806.478819 | 936.5004883 | 14.52928826 | 9.99E−04 | JUCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 2S4_f16:4 | | | | | | | | |
| K@1[229.162 | 0542:3 | | | | | | | | |
| 9]; Oxidation | | | | | | | | | |
| of | | | | | | | | | |
| M@13[15.9949] | | | | | | | | | |
| TMT 10-plex | 08CPTAC_ | 3 | 2806.491354 | −4.466436503 | 2806.478819 | 936.5043945 | 13.61074709 | 9.99E−04 | UCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 2S4_f17: | | | | | | | | |
| K@1[229.162 | 41363: | | | | | | | | |
| 9]; Oxidation | 3 | | | | | | | | |
| of | | | | | | | | | |
| M@13[15.9949] | | | | | | | | | |
| TMT 10-plex | 06CPTAC_ | 4 | 2790.478951 | 1.775262321 | 2790.483905 | 698.6270142 | 13.01749246 | 0.00999001 | UCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 2S2_f05: | | | | | | | | |
| K@1[229.1629] | 20045: | | | | | | | | |
| | 4 | | | | | | | | |
| TMT 10-plex | 17CPTAC_ | 3 | 2790.474936 | 3.214094163 | 2790.483905 | 931.1655884 | 12.5141614 | 0.00999001 | UCEC |
| of peptide N- | UCEC_W_ | | | | | | | | |
| term@0[229. | PNNL_20 | | | | | | | | |
| 1629]; TMT | 181015_B | | | | | | | | |
| 10-plex of K@ | 5126: | | | | | | | | |
| 1[229.1629] | 35S1_f12: | | | | | | | | |
| | 3 | | | | | | | | |
| TMT 10-plex | 05CPTAC_ | 3 | 2790.473288 | 3.804654536 | 2790.483905 | 931.1650391 | 12.45779484 | 0.00999001 | UCEC |
| of peptide N- | UCEC_W_ | | | | | | | | |
| term@0[229. | PNNL_20 | | | | | | | | |
| 1629]; TMT | 170922_B | | | | | | | | |
| 10-plex of | 2S1_f12: | | | | | | | | |
| K@1[229.162 | 40137: | | | | | | | | |
| 9] | 3 | | | | | | | | |
| TMT 10-plex | 09CPTAC_ | 3 | 2806.481283 | −0.878025034 | 2806.478819 | 936.5010376 | 12.34245156 | 0.006993007 | UCEC |
| of peptide N- | UCEC_W_ | | | | | | | | |
| term@0[229. | PNNL_20 | | | | | | | | |
| 1629]; | 180222_B | | | | | | | | |
| TMT | 3S1_f16: | | | | | | | | |
| 10-plex of | 39914: | | | | | | | | |
| K@1[229.162 | 3 | | | | | | | | |
| 9]; | | | | | | | | | |
| Oxidation | | | | | | | | | |
| of | | | | | | | | | |
| M@13[15.9949] | | | | | | | | | |
| TMT 10-plex | 10CPTAC_ | 3 | 2806.488058 | −3.292047296 | 2806.478819 | 936.5032959 | 12.06775055 | 0.007992008 | UCEC |
| of peptide N- | UCEC_W_ | | | | | | | | |
| term@0[229.1629]; | PNNL_20 | | | | | | | | |
| TMT | 180222_B | | | | | | | | |
| 10-plex of | 9729: | | | | | | | | |
| K@1[229.1629]; | 33S2_ | | | | | | | | |
| Oxidation | f16: | | | | | | | | |
| of | 3 | | | | | | | | |
| M@13[15.9949] | | | | | | | | | |
| TMT 10-plex | 18CPTAC_ | 3 | 2806.497946 | −6.815214919 | 2806.478819 | 936.5065918 | 17.48129513 | 9.99E−04 | LUAD |
| of peptide N- | LUAD_W_ | | | | | | | | |
| term@0 | BI_20180 | | | | | | | | |
| [229.1629]; | 726_KL_ | | | | | | | | |
| TMT | f19: | | | | | | | | |
| 10-plex of | 37596: | | | | | | | | |
| K@1[229.1629]; | 3 | | | | | | | | |
| Oxidation | | | | | | | | | |
| of | | | | | | | | | |
| M@13[15.9949] | | | | | | | | | |
| TMT 10-plex | 23CPTAC_ | 3 | 2806.49172 | −4.596924193 | 2806.478819 | 936.5045166 | 17.47458706 | 9.99E−04 | LUAD |
| of peptide N- | LUAD_W_ | | | | | | | | |
| term@0[229. | BI_20180 | | | | | | | | |
| 1629]; | 822_KR_ | | | | | | | | |
| TMT | f19: | | | | | | | | |
| 10-plex of | 32708: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 19CPTAC_ | 3 | 2806.494467 | −5.575581866 | 2806.478819 | 936.5054321 | 17.41006334 | 9.99E−04 | LUAD |
| of peptide N- | LUAD_W_ | | | | | | | | |
| term@0 | BI_20180 | | | | | | | | |
| [229.1629]; | 730_KL_f1 | | | | | | | | |
| TMT | 9: | | | | | | | | |
| 10-plex of | 34556: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 22CPTAC_ | 3 | 2806.479269 | −0.160342741 | 2806.478819 | 936.5003662 | 14.66673191 | 9.99E−04 | LUAD |
| of peptide N- | LUAD_W_ | | | | | | | | |
| term@0[229. | BI_20180 | | | | | | | | |
| 1629]; | 820_KR_f | | | | | | | | |
| TMT | 3 | | | | | | | | |
| 10-plex of | 20: | | | | | | | | |
| K@1 | 35166: | | | | | | | | |
| [229.1629]; | | | | | | | | | |
| Oxidation of | | | | | | | | | |
| M@13 | | | | | | | | | |
| [15.9949] | | | | | | | | | |
| TMT 10-plex | 07CPTAC_ | 3 | 2806.481833 | −1.073756569 | 2806.478819 | 936.5012207 | 14.64574741 | 0.002997003 | LUAD |
| of peptide N- | LUAD_W_ | | | | | | | | |
| term@0 | BI_20180 | | | | | | | | |
| [229.1629]; | 602_KR_f | | | | | | | | |
| TMT | 20: | | | | | | | | |
| 10-plex of | 33243: | | | | | | | | |
| K@1[229.162 | 3 | | | | | | | | |
| 9]; | | | | | | | | | |
| Oxidation | | | | | | | | | |
| of | | | | | | | | | |
| M@13 | | | | | | | | | |
| [15.9949] | | | | | | | | | |
| TMT 10-plex | 25CPTAC_ | 3 | 2806.49227 | −4.792655727 | 2806.478819 | 936.5046997 | 14.63317617 | 0.001998002 | LUAD |
| of peptide N- | LUAD_W_ | | | | | | | | |
| term@0[229. | BI_20180 | | | | | | | | |
| 1629]; | 901_KR_f | | | | | | | | |
| TMT | 21: | | | | | | | | |
| 10-plex of | 32863: | | | | | | | | |
| K@1 | 3 | | | | | | | | |
| [229.1629]; | | | | | | | | | |
| Oxidation of | | | | | | | | | |
| M@13 | | | | | | | | | |
| [15.9949] | | | | | | | | | |
| TMT 10-plex | 12CPTAC_ | 3 | 2806.471029 | 2.775630279 | 2806.478819 | 936.4976196 | 14.62944939 | 9.99E−04 | LUAD |
| of peptide N- | LUAD_W_ | | | | | | | | |
| term@0 | BI_20180 | | | | | | | | |
| [229.1629]; | 912_KL_f2 | | | | | | | | |
| TMT | 0: | | | | | | | | |
| 10-plex of | 34890: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 06CPTAC_ | 3 | 2806.484946 | −2.182901932 | 2806.478819 | 936.5022583 | 14.62673521 | 0.002997003 | LUAD |
| of peptide N- | LUAD_W_ | | | | | | | | |
| term@0[229. | BI_20180 | | | | | | | | |
| 1629]; | 530_KR_f | | | | | | | | |
| TMT | 22: | | | | | | | | |
| 10-plex of | 33024: | | | | | | | | |
| K@1 | 3 | | | | | | | | |
| [229.1629]; | | | | | | | | | |
| Oxidation | | | | | | | | | |
| of | | | | | | | | | |
| M@13[15.99 | | | | | | | | | |
| 49] | | | | | | | | | |
| TMT 10-plex | 01CPTAC_ | 3 | 2806.498312 | −6.945702609 | 2806.478819 | 936.5067139 | 14.62599993 | 9.99E−04 | LUAD |
| of peptide N- | LUAD_W_ | | | | | | | | |
| term@0[229. | BI_20180 | | | | | | | | |
| 1629]; | 515_KR_f | | | | | | | | |
| TMT | 20: | | | | | | | | |
| 10-plex of | 32991: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 02CPTAC_ | 3 | 2806.485129 | −2.248145778 | 2806.478819 | 936.5023193 | 14.61999828 | 0.001998002 | LUAD |
| of peptide N- | LUAD_W_ | | | | | | | | |
| term@0 | BI_20180 | | | | | | | | |
| [229.1629]; | 518_KR_f | | | | | | | | |
| TMT | 20: | | | | | | | | |
| 10-plex of K@1 | 33028: | | | | | | | | |
| [229.1629]; | 3 | | | | | | | | |
| Oxidation | | | | | | | | | |
| of | | | | | | | | | |
| M@13[15.9949] | | | | | | | | | |
| TMT 10-plex | 15CPTAC_ | 3 | 2806.499228 | −7.271921833 | 2806.478819 | 936.507019 | 14.56936016 | 0.002997003 | LUAD |
| of peptide N- | LUAD_W_ | | | | | | | | |
| term@0[229. | BI_20180 | | | | | | | | |
| 1629]; | 714_KL_f1 | | | | | | | | |
| TMT | 9: | | | | | | | | |
| 10-plex of | 36783: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 12CPTAC_ | 3 | 2806.482382 | −1.269488104 | 2806.478819 | 936.5014038 | 14.55973951 | 0.001998002 | LUAD |
| of peptide N- | LUAD_W_ | | | | | | | | |
| term@0 | BI_20180 | | | | | | | | |
| [229.1629]; | 912_KL_f2 | | | | | | | | |
| TMT | 1: | | | | | | | | |
| 10-plex of | 35032: | | | | | | | | |
| K@1 | 3 | | | | | | | | |
| [229.1629]; | | | | | | | | | |
| Oxidation | | | | | | | | | |
| of | | | | | | | | | |
| M@13 | | | | | | | | | |
| [15.9949] | | | | | | | | | |
| TMT 10-plex | 09CPTAC_ | 3 | 2806.490988 | −4.335948814 | 2806.478819 | 936.5042725 | 14.55552758 | 0.002997003 | LUAD |
| of peptide N- | LUAD_W_ | | | | | | | | |
| term@0 | BI_20180 | | | | | | | | |
| [229.1629]; | 620_KR_f | | | | | | | | |
| TMT | 22: | | | | | | | | |
| 10-plex of | 37641: | | | | | | | | |
| K@1[229.1629]; | 3 | | | | | | | | |
| Oxidation of | | | | | | | | | |
| M@13[15.9949] | | | | | | | | | |
| TMT 10-plex | 11CPTAC_ | 3 | 2806.494101 | −5.445094177 | 2806.478819 | 936.5053101 | 14.54838704 | 0.001998002 | LUAD |
| of peptide N- | LUAD_W_ | | | | | | | | |
| term@0 | BI_20180 | | | | | | | | |
| [229.1629]; | 905_KR_f | | | | | | | | |
| TMT | 21: | | | | | | | | |
| 10-plex of | 33179: | | | | | | | | |
| K@1 | 3 | | | | | | | | |
| [229.1629]; | | | | | | | | | |
| Oxidation of | | | | | | | | | |
| M@13[15.9949] | | | | | | | | | |
| TMT 10-plex | 02CPTAC_ | 3 | 2790.474936 | 3.214094163 | 2790.483905 | 931.1655884 | 14.04976248 | 0.001998002 | LUAD |
| of peptide N- | LUAD_W_ | | | | | | | | |
| term@0 | BI_20180 | | | | | | | | |
| [229.1629]; | 518_KR_f | | | | | | | | |
| TMT | 16: | | | | | | | | |
| 10-plex of | 31970: | | | | | | | | |
| K@1[229.1629] | 3 | | | | | | | | |
| TMT 10-plex | 20CPTAC_ | 3 | 2806.497946 | −6.815214919 | 2806.478819 | 936.5065918 | 13.77365145 | 9.99E−04 | LUAD |
| of peptide N- | LUAD_W_ | | | | | | | | |
| term@0 | BI_20180 | | | | | | | | |
| [229.1629]; | 809_KR_f | | | | | | | | |
| TMT | 19: | | | | | | | | |
| 10-plex of | 33756: | | | | | | | | |
| K@1 | 3 | | | | | | | | |
| [229.1629]; | | | | | | | | | |
| Oxidation | | | | | | | | | |
| of | | | | | | | | | |
| M@13[15.9949] | | | | | | | | | |
| iTRAQ 4-plex | TCGA_D8 | 4 | 2620.370894 | −3.331198411 | 2620.362165 | 656.1 | 15.50376173 | 0.003996004 | BRCA |
| of | -A13Y_A8- | | | | | | | | |
| K@1 | A076_AO- | | | | | | | | |
| 1[144.102]; | A126_117 | | | | | | | | |
| iTRAQ 4- | C_W_BI_2 | | | | | | | | |
| plex of | 0130617_ | | | | | | | | |
| peptide N- | H- | | | | | | | | |
| term@0 | PM_f03: | | | | | | | | |
| [144.1021] | 32229: | | | | | | | | |
| | 4 | | | | | | | | |
| iTRAQ 4-plex | TCGA_E2- | 3 | 2620.388171 | −9.924358335 | 2620.362165 | 874.47 | 14.88712575 | 0.005994006 | BRCA |
| of | A159_A2- | | | | | | | | |
| K@1 | AOT3_A2- | | | | | | | | |
| [144.1021]; | AOYD_117 | | | | | | | | |
| iTRAQ 4- | C_W_BI_2 | | | | | | | | |
| plex of | 0130823_ | | | | | | | | |
| peptide N- | H- | | | | | | | | |
| term@0 | PM_f14: | | | | | | | | |
| [144.1021] | 38890: | | | | | | | | |
| | 3 | | | | | | | | |
| iTRAQ 4-plex | TCGA_A8- | 4 | 2620.370894 | −3.331198411 | 2620.362165 | 656.1 | 14.76428853 | 0.002997003 | BRCA |
| of | A091_C8- | | | | | | | | |
| K@1 | A12L_A2- | | | | | | | | |
| [144.1021]; | AOEX_117 | | | | | | | | |
| iTRAQ 4- | C_W_BI_2 | | | | | | | | |
| plex of | 0130302_ | | | | | | | | |
| peptide N- | H- | | | | | | | | |
| term@0 | PM_f02: | | | | | | | | |
| [144.1021] | 37693: | | | | | | | | |
| | 4 | | | | | | | | |

Peptide: MYQPIQTYPWMNLSR (SEQ ID NO: 1)

| | | | | | | | | | |
|---|---|---|---|---|---|---|---|---|---|
| TMT 10-plex | 15CPTAC_ | 4 | 2188.078072 | −6.055564979 | 2188.064822 | 548.0267944 | 20.11332548 | 0.00999001 | UCEC |
| of peptide N- | UCEC_W_ | | | | | | | | |
| term@0[229. | PNNL_20 | | | | | | | | |
| 1629]; | 180503_B | | | | | | | | |
| Oxidation | 4S3_f16: | | | | | | | | |
| of | 16238: | | | | | | | | |
| M@1 | 4 | | | | | | | | |
| [15.9949]; | | | | | | | | | |
| Oxidation | | | | | | | | | |
| of | | | | | | | | | |
| M@11 | | | | | | | | | |
| [15.9949] | | | | | | | | | |
| TMT 10-plex | 06CPTAC_ | 3 | 2156.070334 | 2.160858882 | 2156.074993 | 719.6973877 | 15.28010802 | 0.006993007 | UCEC |
| of peptide N- | UCEC_W_ | | | | | | | | |
| term@0 | PNNL_20 | | | | | | | | |
| [229.1629] | 170922_B | | | | | | | | |
| | 2S2_f04: | | | | | | | | |
| | 22881: | | | | | | | | |
| | 3 | | | | | | | | |
| TMT 10-plex | 05CPTAC_ | 2 | 2172.068211 | 0.781070905 | 2172.069907 | 1087.041382 | 13.95864453 | 0.001998002 | UCEC |
| of peptide N- | UCEC_W_ | | | | | | | | |
| term@0 | PNNL_20 | | | | | | | | |
| [229.1629]; | 170922_B | | | | | | | | |
| Oxidation | 2S1_f12: | | | | | | | | |
| of | 43057: | | | | | | | | |
| M@1 | 2 | | | | | | | | |
| [15.9949] | | | | | | | | | |
| TMT 10-plex | 10CPTAC_ | 3 | 2156.070151 | 2.245784254 | 2156.07499 3 | 719.6973267 | 13.76291226 | 0.006993007 | UCEC |
| of peptide N- | UCEC_W_ | | | | | | | | |
| term@0 | PNNL_20 | | | | | | | | |
| [229.1629] | 180222_B | | | | | | | | |
| | 3S2_f23: | | | | | | | | |
| | 25040: | | | | | | | | |
| | 3 | | | | | | | | |
| TMT 10-plex | 04CPTAC_ | 4 | 2156.070259 | 2.195328172 | 2156.074993 | 540.0248413 | 13.47211538 | 0.00999001 | UCEC |
| of peptide N- | UCEC_W_ | | | | | | | | |
| term@0 | PNNL_20 | | | | | | | | |
| [229.1629] | 170922_B | | | | | | | | |
| | 1S4_f09: | | | | | | | | |
| | 32223: | | | | | | | | |
| | 4 | | | | | | | | |
| TMT 10-plex | 15CPTAC_ | 4 | 2188.085884 | −9.626071631 | 2188.064822 | 548.0287476 | 19.77621319 | 0.005994006 | LUAD |
| of peptide N- | LUAD_W_ | | | | | | | | |
| term@0[229. | BI_20180 | | | | | | | | |
| 1629]; | 714_KL_ | | | | | | | | |
| Oxidation | f19: | | | | | | | | |
| of | 16534: | | | | | | | | |
| M@1 | 4 | | | | | | | | |
| [15.9949]; | | | | | | | | | |
| Oxidation | | | | | | | | | |
| of | | | | | | | | | |
| M@11 | | | | | | | | | |
| [15.9949] | | | | | | | | | |
| TMT 10-plex | 22CPTAC_ | 4 | 2188.07856 | −6.278721644 | 2188.064822 | 548.0269165 | 17.57720633 | 0.00999001 | LUAD |
| of peptide N- | LUAD_W_ | | | | | | | | |
| term@0[229. | BI_20180 | | | | | | | | |
| 1629]; | 820_KR_f | | | | | | | | |
| Oxidation of | 20: | | | | | | | | |
| M@1 | 18029: | | | | | | | | |
| [15.9949]; | 4 | | | | | | | | |
| Oxidation | | | | | | | | | |
| of | | | | | | | | | |
| M@11 | | | | | | | | | |
| [15.9949] | | | | | | | | | |
| TMT 10-plex | 02CPTAC_ | 3 | 2156.087362 | −5.737200625 | 2156.074993 | 719.703064 | 16.52488406 | 0.003996004 | LUAD |
| of peptide N- | LUAD_W_ | | | | | | | | |
| term@0 | BI_20180 | | | | | | | | |
| [229.1629] | 518_KR_f | | | | | | | | |
| | 19: | | | | | | | | |
| | 17455: | | | | | | | | |
| | 3 | | | | | | | | |
| TMT 10-plex | 05CPTAC_ | 3 | 2156.089377 | −6.671379707 | 2156.074993 | 719.7037354 | 15.647653 | 0.002997003 | LUAD |
| of peptide N- | LUAD_W_ | | | | | | | | |
| term@0 | BI_20180 | | | | | | | | |
| [229.1629] | 527_KR_f | | | | | | | | |
| | 02: | | | | | | | | |
| | 34310: | | | | | | | | |
| | 3 | | | | | | | | |
| TMT 10-plex | 14CPTAC_ | 3 | 2156.068136 | 3.179963334 | 2156.074993 | 719.6966553 | 14.6276659 | 0.006993007 | LUAD |
| of peptide N- | LUAD_W_ | | | | | | | | |
| term@0 | BI_20180 | | | | | | | | |
| [229.1629] | 711_KL_f2 | | | | | | | | |
| | 3: | | | | | | | | |
| | 42437: | | | | | | | | |
| | 3 | | | | | | | | |
| TMT 10-plex | 09CPTAC_ | 3 | 2156.061178 | 71276.40434 | 2156.074993 | 719.6943359 | 14.26148765 | 0.007992008 | LUAD |
| of peptide N- | LUAD_W_ | | | | | | | | |
| term@0 | BI_20180 | | | | | | | | |
| [229.1629] | 620_KR_f01: | | | | | | | | |
| | 44535: | | | | | | | | |
| | 3 | | | | | | | | |
| TMT 10-plex | 22CPTAC_ | 3 | 2172.079062 | −4.214612962 | 2172.069907 | 725.0336304 | 13.65996514 | 0.005994006 | LUAD |
| of peptide N- | LUAD_W_ | | | | | | | | |
| term@0 | BI_20180 | | | | | | | | |
| [229.1629]; | 820_KR_ | | | | | | | | |
| Oxidation of | f08: | | | | | | | | |
| M@1 | 5175: | | | | | | | | |
| [15.9949] | 3 | | | | | | | | |
| iTRAQ 4-plex | TCGA_ | 3 | 2215.118171 | −0.896220883 | 2215.116185 | 739.38 | 22.56300467 | 9.99E−04 | BRCA |
| of peptide N- | AR- | | | | | | | | |
| term@0 | AOTT_AR- | | | | | | | | |
| [144.1021]; | A1AQ_AO | | | | | | | | |
| iTRAQ | A12B_117 | | | | | | | | |
| 4-plex of | -C_ | | | | | | | | |
| Y@2[144.1021] | W_BI_2 | | | | | | | | |
| | 0131022_ | | | | | | | | |
| | H- | | | | | | | | |
| | PM_ | | | | | | | | |
| | f11: | | | | | | | | |
| | 18656: | | | | | | | | |
| | 3 | | | | | | | | |
| iTRAQ 4-plex | TCGA_AR | 3 | 2375.198171 | 6.311773281 | 2375.213162 | 792.74 | 20.40592608 | 9.99E−04 | BRCA |
| of peptide N- | -AOTR_ | | | | | | | | |
| term@0[144. | AO- | | | | | | | | |
| 1021]; | A030_BH- | | | | | | | | |
| iTRAQ | A18R_117 | | | | | | | | |
| 4-plex of | C_W_BI_2 | | | | | | | | |
| Y@2 | 0130825_H- | | | | | | | | |
| [144.1021]; | PM_f22: | | | | | | | | |
| İTRAQ 4- | 32391: | | | | | | | | |
| plex of | 3 | | | | | | | | |
| Y@8 | | | | | | | | | |
| [144.1021]; | | | | | | | | | |
| Oxidation of | | | | | | | | | |
| M@11 | | | | | | | | | |
| [15.9949] | | | | | | | | | |
| iTRAQ 4-plex | TCGA_A8- | 4 | 2215.130894 | −6.640178366 | 2215.116185 | 554.79 | 20.32976862 | 9.99E−04 | BRCA |
| of peptide N- | A06Z_A2- | | | | | | | | |
| term@0 | AOD1_A2- | | | | | | | | |
| [144.1021]; | AOCM_11 | | | | | | | | |
| iTRAQ | 7C_W_BI_ | | | | | | | | |
| 4-plex of | 20130401_ | | | | | | | | |
| Y@2[144.1021] | H- | | | | | | | | |
| | JQ_f24: | | | | | | | | |
| | 6308: | | | | | | | | |
| | 4 | | | | | | | | |
| iTRAQ 4-plex | TCGA_E2- | 3 | 2087.018171 | −4.376135637 | 2087.009038 | 696.68 | 18.18083197 | 0.003996004 | BRCA |
| of peptide N- | A10A_BH- | | | | | | | | |
| term@0 | A18Q_C8- | | | | | | | | |
| [144.1021]; | A130_117 | | | | | | | | |
| Oxidation of | C_W_BI_2 | | | | | | | | |
| M@1 | 0130222_ | | | | | | | | |
| [15.9949] | H- | | | | | | | | |
| | PM_f20: | | | | | | | | |
| | 37936: | | | | | | | | |
| | 3 | | | | | | | | |
| iTRAQ 4-plex | TCGA_A2- | 3 | 2375.198171 | 6.311773281 | 2375.213162 | 792.74 | 17.80083892 | 9.99E−04 | BRCA |
| of peptide N- | AOYF_BH- | | | | | | | | |
| term@0 | AODD_BH | | | | | | | | |
| [144.1021]; | -AOE9_ | | | | | | | | |
| Oxidation of | 117 | | | | | | | | |
| M@1 | C_W_BI_2 | | | | | | | | |
| [15.9949]; | 0131018_ | | | | | | | | |
| iTRAQ 4- | H- | | | | | | | | |
| plex of | PM_f19: | | | | | | | | |
| Y@2 | 35729: | | | | | | | | |
| [144.1021]; | 3 | | | | | | | | |
| İTRAQ 4- | | | | | | | | | |
| plex of | | | | | | | | | |
| Y@8 | | | | | | | | | |
| [144.1021] | | | | | | | | | |
| iTRAQ 4-plex | TCGA_A2- | 3 | 2375.198171 | 6.311773281 | 2375.213162 | 792.74 | 17.68258049 | 0.002997003 | BRCA |
| of peptide N- | A0D2_C8- | | | | | | | | |
| term@0 | A12U_AR- | | | | | | | | |
| [144.1021]; | A1AS_117 | | | | | | | | |
| Oxidation of | C_W_BI_2 | | | | | | | | |
| M@1 | 0131010_ | | | | | | | | |
| [15.9949]; | H- | | | | | | | | |
| iTRAQ 4- | PM_f20: | | | | | | | | |
| plex of | 37261: | | | | | | | | |
| Y@2 | 3 | | | | | | | | |
| [144.1021]; | | | | | | | | | |
| İTRAQ 4- | | | | | | | | | |
| plex of | | | | | | | | | |
| Y@8 | | | | | | | | | |
| [144.1021] | | | | | | | | | |
| iTRAQ 4-plex | TCGA_AO | 3 | 2087.018171 | −4.376135637 | 2087.009038 | 696.68 | 17.33022178 | 0.003996004 | BRCA |
| of peptide N- | -AOJE_A2- | | | | | | | | |
| term@0 | AOT2_AN- | | | | | | | | |
| [144.1021]; | AOAJ_117 | | | | | | | | |
| Oxidation of | C_W_BI_2 | | | | | | | | |
| M@1 | 0130802_ | | | | | | | | |
| [15.9949] | H- | | | | | | | | |
| | PM_f20: | | | | | | | | |
| | 38643: | | | | | | | | |
| | 3 | | | | | | | | |
| iTRAQ 4-plex | TCGA_A7- | 3 | 2087.018171 | −4.376135637 | 2087.009038 | 696.68 | 17.326379 | 0.001998002 | BRCA |
| of peptide N- | AOCJ_AO- | | | | | | | | |
| term@0 | A12F_A2- | | | | | | | | |
| [144.1021]; | AOYL_117 | | | | | | | | |
| Oxidation of | C_W_BI_2 | | | | | | | | |
| M@1 | 0130805_ | | | | | | | | |
| [15.9949] | H- | | | | | | | | |
| | PM_f20: | | | | | | | | |
| | 37564: | | | | | | | | |
| | 3 | | | | | | | | |
| iTRAQ 4-plex | TCGA_A2- | 3 | 2087.018171 | −4.376135637 | 2087.009038 | 696.68 | 17.30956675 | 0.001998002 | BRCA |
| of peptide N- | AOT6_E2- | | | | | | | | |
| term@0 | A158_E2- | | | | | | | | |
| [144.1021]; | A15A_117 | | | | | | | | |
| Oxidation of | C_W_BI_2 | | | | | | | | |
| M@1 | 0130918_ | | | | | | | | |
| [15.9949] | H- | | | | | | | | |
| | PM_f20: | | | | | | | | |
| | 35857: | | | | | | | | |
| | 3 | | | | | | | | |
| iTRAQ 4-plex | TCGA_AN | 3 | 2087.018171 | −4.376135637 | 2087.009038 | 696.68 | 17.29442272 | 0.004995005 | BRCA |
| of peptide N- | AOFL_BH- | | | | | | | | |
| term@0 | AODG_AN | | | | | | | | |
| [144.1021]; | -AOAS_ | | | | | | | | |
| Oxidation of | 117 | | | | | | | | |
| M@1[15.994 | C_W_BI_2 | | | | | | | | |
| 9] | 0130726_ | | | | | | | | |
| | H- | | | | | | | | |
| | PM_f19: | | | | | | | | |
| | 34822: | | | | | | | | |
| | 3 | | | | | | | | |
| iTRAQ 4-plex | TCGA_AO | 4 | 2231.130894 | −8.871878685 | 2231.1111 | 558.79 | 17.27007557 | 0.001998002 | BRCA |
| of peptide N- | -A12E_A8- | | | | | | | | |
| term@0[144. | A06N_A2- | | | | | | | | |
| 1021]; | AOT1_117 | | | | | | | | |
| iTRAQ | C_W_BI_2 | | | | | | | | |
| 4-plex of | 0130909_ | | | | | | | | |
| Y@2[144.102 | H- | | | | | | | | |
| 1]; | PM_f24: | | | | | | | | |
| Oxidation | 6484: | | | | | | | | |
| of | 4 | | | | | | | | |
| M@11 | | | | | | | | | |
| [15.9949] | | | | | | | | | |
| iTRAQ 4-plex | TCGA_C8 | 3 | 2087.018171 | −4.376135637 | 2087.009038 | 696.68 | 17.11676546 | 0.001998002 | BRCA |
| of peptide N- | -A12P_ | | | | | | | | |
| term@0 | BH- | | | | | | | | |
| [144.1021]; | AOC1_A2- | | | | | | | | |
| Oxidation of | AOEY_117 | | | | | | | | |
| M@1 | C_W_BI_2 | | | | | | | | |
| [15.9949] | 0130622_ | | | | | | | | |
| | H- | | | | | | | | |
| | PM_f18: | | | | | | | | |
| | 34865: | | | | | | | | |
| | 3 | | | | | | | | |
| iTRAQ 4-plex | TCGA_E2- | 3 | 2087.018171 | −4.376135637 | 2087.009038 | 696.68 | 17.05213208 | 0.002997003 | BRCA |
| of peptide N- | A159_A2- | | | | | | | | |
| term@0 | AOT3_A2- | | | | | | | | |
| [144.1021]; | AOYD_117 | | | | | | | | |
| Oxidation of | C_W_BI_2 | | | | | | | | |
| M@1 | 0130823_ | | | | | | | | |
| [15.9949] | H- | | | | | | | | |
| | PM_f19: | | | | | | | | |
| | 38 | | | | | | | | |
| | 057: | | | | | | | | |
| | 3 | | | | | | | | |
| iTRAQ 4-plex | TCGA_AO | 4 | 2215.130894 | −6.640178366 | 2215.116185 | 554.79 | 17.04986473 | 0.003996004 | BRCA |
| of peptide N- | -AOJM_ | | | | | | | | |
| term@0 | C8- | | | | | | | | |
| [144.1021]; | A12V_A8- | | | | | | | | |
| iTRAQ | A08G_117 | | | | | | | | |
| 4-plex of | C_W_BI_2 | | | | | | | | |
| Y@2 | 0130927_ | | | | | | | | |
| [144.1021] | H- | | | | | | | | |
| | PM_f24: | | | | | | | | |
| | 6719: | | | | | | | | |
| | 4 | | | | | | | | |
| iTRAQ 4-plex | TCGA_A2- | 3 | 2087.018171 | −4.376135637 | 2087.009038 | 696.68 | 17.02998754 | 0.004995005 | BRCA |
| of peptide N- | AOT7_C8- | | | | | | | | |
| term@0 | A12Q_A8- | | | | | | | | |
| [144.1021]; | A079_117 | | | | | | | | |
| Oxidation of | C_W_BI_2 | | | | | | | | |
| M@1 | 0130820_ | | | | | | | | |
| [15.9949] | H- | | | | | | | | |
| | PM_f19: | | | | | | | | |
| | 37885: | | | | | | | | |
| | 3 | | | | | | | | |
| iTRAQ 4-plex | TCGA_A2-1 | 3 | 2087.018171 | −4.376135637 | 2087.009038 | 696.68 | 16.98123963 | 0.006993007 | BRCA |
| of peptide N- | AODO_BH- | | | | | | | | |
| term@0[144. | AOHK_C8- | | | | | | | | |
| 1021]; | A12T_117 | | | | | | | | |
| Oxidation of | C_W_BI_2 | | | | | | | | |
| M@1[15.9949] | 0130326_ | | | | | | | | |
| | H- | | | | | | | | |
| | JQ_f20: | | | | | | | | |
| | 32127: | | | | | | | | |
| | 3 | | | | | | | | |
| iTRAQ 4-plex | TCGA_D8 | 3 | 2071.028171 | −6.782983287 | 2071.014123 | 691.35 | 16.92507984 | 9.99E−04 | BRCA |
| of peptide N- | -A13Y_A8- | | | | | | | | |
| term@0 | A076_AO- | | | | | | | | |
| [144.1021] | A126_117 | | | | | | | | |
| | C_W_BI_2 | | | | | | | | |
| | 0130617_ | | | | | | | | |
| | H- | | | | | | | | |
| | PM_f09: | | | | | | | | |
| | 42446: | | | | | | | | |
| | 3 | | | | | | | | |
| iTRAQ 4-plex | TCGA_AO | 3 | 2087.018171 | −4.376135637 | 2087.009038 | 696.68 | 16.83702318 | 0.005994006 | BRCA |
| of peptide N- | - | | | | | | | | |
| term@0[144. | A12D_C8- | | | | | | | | |
| 1021]; | A131_AO- | | | | | | | | |
| Oxidation of | A12B_117 | | | | | | | | |
| M@1[15.9949] | C_W_BI_2 | | | | | | | | |
| | 0130208_ | | | | | | | | |
| | H- | | | | | | | | |
| | PM_f20: | | | | | | | | |
| | 36958: | | | | | | | | |
| | 3 | | | | | | | | |
| iTRAQ 4-plex | TCGA_C8 | 3 | 2087.018171 | −4.376135637 | 2087.009038 | 696.68 | 16.82217829 | 0.004995005 | BRCA |
| of peptide N- | -A138_E2- | | | | | | | | |
| term@0[144. | A154_BH- | | | | | | | | |
| 1021]; | AOBZ_117 | | | | | | | | |
| Oxidation of | C_W_BI_2 | | | | | | | | |
| M@1[15.9949] | 0130225_ | | | | | | | | |
| | H- | | | | | | | | |
| | PM_f20: | | | | | | | | |
| | 36784: | | | | | | | | |
| | 3 | | | | | | | | |
| iTRAQ 4-plex | TCGA_A7- | 3 | 2087.018171 | −4.376135637 | 2087.009038 | 696.68 | 16.79265305 | 0.003996004 | BRCA |
| of peptide N- | AOCD_C8- | | | | | | | | |
| term@0[144. | A12W_AN | | | | | | | | |
| 1021]; | -AOAL_ | | | | | | | | |
| Oxidation of | 117 | | | | | | | | |
| M@1[15.9949] | C_W_BI_2 | | | | | | | | |
| | 0130913_ | | | | | | | | |
| | H- | | | | | | | | |
| | PM_f19: | | | | | | | | |
| | 36 | | | | | | | | |
| | 048: | | | | | | | | |
| | 3 | | | | | | | | |
| iTRAQ 4-plex | CPTAC_2 | 3 | 2359.208171 | 4.271409576 | 2359.21824 8 | 787.41 | 16.76729881 | 0.007992008 | BRCA |
| of peptide N- | 63d3f- | | | | | | | | |
| term@0[144. | I_blcdb9- | | | | | | | | |
| 1021]; | I_ | | | | | | | | |
| iTRAQ | c4155b- | | | | | | | | |
| 4-plex of | C_117C_ | | | | | | | | |
| Y@2 | W_BI_201 | | | | | | | | |
| [144.1021]; | 072: | | | | | | | | |
| iTRAQ 4- | 340417_ | | | | | | | | |
| plex of | H- | | | | | | | | |
| Y@8[144.1021] | PM_f05: | | | | | | | | |
| | 31 | | | | | | | | |
| iTRAQ 4-plex | TCGA_BH | 3 | 2215.118171 | −0.896220883 | 2215.116185 | 739.38 | 16.76494205 | 0.001998002 | BRCA |
| of peptide N- | -A18U_ | | | | | | | | |
| term@0[144. | A2- | | | | | | | | |
| 1021]; | AOYI_A2- | | | | | | | | |
| iTRAQ | AOEQ_117 | | | | | | | | |
| 4-plex of | C_W_BI_2 | | | | | | | | |
| Y@2[144.1021] | 0130405_H- | | | | | | | | |
| | JQ_f07: | | | | | | | | |
| | 31507: | | | | | | | | |
| | 3 | | | | | | | | |
| iTRAQ 4-plex | TCGA_A2- | 3 | 2375.198171 | 6.311773281 | 2375.213162 | 792.74 | 16.74790443 | 0.003996004 | BRCA |
| of peptide N- | AOT7_C8- | | | | | | | | |
| term@0 | A12Q_A8- | | | | | | | | |
| [144.1021]; | A079_117 | | | | | | | | |
| iTRAQ | C_W_BI_2 | | | | | | | | |
| 4-plex of | 0130820_ | | | | | | | | |
| Y@2 | H- | | | | | | | | |
| [144.1021]; | PM_f20: | | | | | | | | |
| iTRAQ 4- | 38900: | | | | | | | | |
| plex of | 3 | | | | | | | | |
| Y@8 | | | | | | | | | |
| 1[144.102]; | | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_BH | 4 | 2215.130894 | −6.640178366 | 2215.116185 | 554.79 | 16.73149302 | 9.99E−04 | BRCA |
| of peptide N- | -A18V_A7- | | | | | | | | |
| term@0 | A13F_BH- | | | | | | | | |
| [144.1021]; | AOE1_117 | | | | | | | | |
| iTRAQ | C_W_BI_2 | | | | | | | | |
| 4-plex of | 0130520_ | | | | | | | | |
| Y@2 | H- | | | | | | | | |
| [144.1021] | PM_f12: | | | | | | | | |
| | 7645: | | | | | | | | |
| | 4 | | | | | | | | |
| iTRAQ 4-plex | TCGA_BH | 3 | 2087.018171 | 314.3761356 | 2087.009038 | 696.68 | 16.72737988 | 0.003996004 | BRCA |
| of peptide N- | -A18U_ | | | | | | | | |
| term@0[144. | A2- | | | | | | | | |
| 1021]; | AOYI_A2- | | | | | | | | |
| Oxidation of | AOEQ_117 | | | | | | | | |
| M@11[15.9949] | C_W_BI_ | | | | | | | | |
| | 2013045_ | | | | | | | | |
| | H- | | | | | | | | |
| | JQ_f19: | | | | | | | | |
| | 32085: | | | | | | | | |
| | 3 | | | | | | | | |
| iTRAQ 4-plex | TCGA_A8- | 3 | 2087.018171 | 314.3761356 | 2087.009038 | 696.68 | 16.72722145 | 0.006993007 | BRCA |
| of peptide N- | A091_C8- | | | | | | | | |
| term@0[144. | A12L_A2- | | | | | | | | |
| 1021 ]; | AOEX_117 | | | | | | | | |
| Oxidation of | C_W_BI_2 | | | | | | | | |
| M@1[15.9949] | 0130302_ | | | | | | | | |
| | H- | | | | | | | | |
| | PM_f20: | | | | | | | | |
| | 37086: | | | | | | | | |
| | 3 | | | | | | | | |
| iTRAQ 4-plex | TCGA_AO | 3 | 2087.018171 | 314.3761356 | 2087.009038 | 696.68 | 16.71325405 | 0.004995005 | BRCA |
| of peptide N- | - | | | | | | | | |
| term@0[144. | A12D_AN- | | | | | | | | |
| 1021]; | A04A_BH- | | | | | | | | |
| Oxidation of | AOAV_117 | | | | | | | | |
| M@1[15.9949] | C_W_BI_2 | | | | | | | | |
| | 0130310_ | | | | | | | | |
| | H- | | | | | | | | |
| | PM_f20: | | | | | | | | |
| | 34513: | | | | | | | | |
| | 3 | | | | | | | | |
| iTRAQ 4-plex | TCGA_E2- | 3 | 2087.018171 | 314.3761356 | 2087.009038 | 696.68 | 16.71299364 | 0.005994006 | BRCA |
| of peptide N- | A10A_BH- | | | | | | | | |
| term@0[144. | A18Q_C8- | | | | | | | | |
| 1021]; | A130_117 | | | | | | | | |
| Oxidation of | C_W_BI_2 | | | | | | | | |
| M@1[15.9949] | 0130222_ | | | | | | | | |
| | H- | | | | | | | | |
| | PM_f19: | | | | | | | | |
| | 38506: | | | | | | | | |
| | 3 | | | | | | | | |
| iTRAQ 4-plex | CPTAC_2 | 3 | 2359.208171 | 4.271409576 | 2359.218248 | 787.41 | 16.70103487 | 0.004995005 | BRCA |
| of peptide N- | 63d3f- | | | | | | | | |
| term@0[144. | I_blcdb9- | | | | | | | | |
| 1021]; | I_c4155b- | | | | | | | | |
| iTRAQ | C_117C_ | | | | | | | | |
| 4-plex of | 813: | | | | | | | | |
| Y@2[144.102 | 3 | | | | | | | | |
| 1]; | W_BI_2014 | | | | | | | | |
| iTRAQ 4- | 0417_H- | | | | | | | | |
| plex of | PM_f04: | | | | | | | | |
| Y@8[144.1021] | 29 | | | | | | | | |
| iTRAQ 4-plex | CPTAC_2 | 3 | 2375.198171 | 6.311773281 | 2375.213162 | 792.74 | 16.69422113 | 0.003996004 | BRCA |
| of peptide N- | 63d3f- | | | | | | | | |
| term@0[144. | I_blcdb9- | | | | | | | | |
| 1021]; | _c4155b- | | | | | | | | |
| Oxidation of | C_117C_ | | | | | | | | |
| M@1 | W_BI_201 | | | | | | | | |
| [15.9949]; | 208: | | | | | | | | |
| iTRAQ 4- | 340417_H- | | | | | | | | |
| plex of Y@ | PM_f15: | | | | | | | | |
| 2[144.1021]; | 30 | | | | | | | | |
| iTRAQ 4- | | | | | | | | | |
| plex of | | | | | | | | | |
| Y@8[144.1021] | | | | | | | | | |
| iTRAQ 4-plex | TCGA_A7- | 3 | 2071.028171 | 316.7829833 | 2071.014123 | 691.35 | 16.67303881 | 0.004995005 | BRCA |
| of peptide N- | AOCE_BH- | | | | | | | | |
| term@0[144. | AOCO_A2- | | | | | | | | |
| 1021] | AOYC_117 | | | | | | | | |
| | C_W_BI_2 | | | | | | | | |
| | 0130524_ | | | | | | | | |
| | H- | | | | | | | | |
| | PM_f20: | | | | | | | | |
| | 29894: | | | | | | | | |
| | 3 | | | | | | | | |
| iTRAQ 4-plex | CPTAC_2 | 3 | 2359.208171 | 4.271409576 | 2359.218248 | 787.41 | 16.65815347 | 0.003996004 | BRCA |
| of peptide N- | 63d3f- | | | | | | | | |
| term@0[144. | I_blcdb9- | | | | | | | | |
| 1021]; | I_c4155b- | | | | | | | | |
| iTRAQ | C_117C_ | | | | | | | | |
| 4-plex of | 758: | | | | | | | | |
| Y@2[144.102 | 3 | | | | | | | | |
| 1]; | W_BI_201 | | | | | | | | |
| iTRAQ 4- | 40417_H- | | | | | | | | |
| plex of Y | PM_f14: | | | | | | | | |
| @8[144.1021] | 31758:3 | | | | | | | | |
| iTRAQ 4-plex | TCGA_AO | 3 | 2087.018171 | 314.3761356 | 2087.009038 | 696.68 | 16.59598794 | 9.99E−04 | BRCA |
| of peptide N- | - | | | | | | | | |
| term@0[144. | A12D_AN- | | | | | | | | |
| 1021]; | A04A_BH- | | | | | | | | |
| Oxidation of | AOAV_117 | | | | | | | | |
| M@1[15.9949] | C_W_BI_2 | | | | | | | | |
| | 0130310_ | | | | | | | | |
| | H- | | | | | | | | |
| | PM_f19: | | | | | | | | |
| | 35306: | | | | | | | | |
| | 3 | | | | | | | | |
| iTRAQ 4-plex | TCGA_C8 | 3 | 2087.018171 | 314.3761356 | 2087.009038 | 696.68 | 16.46472766 | 0.005994006 | BRCA |
| of peptide N- | -A138_E2- | | | | | | | | |
| term@0[144. | A154_BH- | | | | | | | | |
| 1021]; | AOBZ_117 | | | | | | | | |
| Oxidation of | C_W_BI_2 | | | | | | | | |
| M@1[15.9949] | 0130225_ | | | | | | | | |
| | H- | | | | | | | | |
| | PM_f19: | | | | | | | | |
| | 37778: | | | | | | | | |
| | 3 | | | | | | | | |
| iTRAQ 4-plex | TCGA_A2- | 3 | 2375.198171 | 6.311773281 | 2375.213162 | 792.74 | 16.24435955 | 0.001998002 | BRCA |
| of peptide N- | AOYM_BH | | | | | | | | |
| term@0[144. | - | | | | | | | | |
| 1021]; | AOC7_A2- | | | | | | | | |
| Oxidation of | AOSX_117 | | | | | | | | |
| M@1[15.9949]; | C_W_BI_2 | | | | | | | | |
| | 0131025_ | | | | | | | | |
| | H- | | | | | | | | |
| iTRAQ 4-plex of | PM_f16: | | | | | | | | |
| Y@2 | 22 | | | | | | | | |
| [144.1021]; | 452: | | | | | | | | |
| iTRAQ 4- | 3 | | | | | | | | |
| plex of | | | | | | | | | |
| Y@8[144.1021] | | | | | | | | | |
| iTRAQ 4-plex | TCGA_BH | 3 | 2375.198171 | 6.311773281 | 2375.213162 | 792.74 | 15.95345862 | 0.004995005 | BRCA |
| of peptide N- | -A18U_ | | | | | | | | |
| term@0 | A2- | | | | | | | | |
| [144.1021]; | AOYI_A2- | | | | | | | | |
| iTRAQ | AOEQ_117 | | | | | | | | |
| 4-plex of | C_W_BI_2 | | | | | | | | |
| Y@2 | 0130405_ | | | | | | | | |
| [144.1021]; | H- | | | | | | | | |
| iTRAQ 4- | JQ_f16: | | | | | | | | |
| plex of | 18542: | | | | | | | | |
| Y@8 | 3 | | | | | | | | |
| [144.1021]; | | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_AR | 3 | 2071.028171 | 316.7829833 | 2071.014123 | 691.35 | 15.84057791 | 9.99E−04 | BRCA |
| of peptide N- | -AOTT_ | | | | | | | | |
| term@0 | AR- | | | | | | | | |
| [144.1021] | A1AQ_ | | | | | | | | |
| | AO- | | | | | | | | |
| | A12B_117 | | | | | | | | |
| | C_W_BI_2 | | | | | | | | |
| | 0131022_ | | | | | | | | |
| | H- | | | | | | | | |
| | PM_f09: | | | | | | | | |
| | 36167: | | | | | | | | |
| | 3 | | | | | | | | |
| iTRAQ 4-plex | TCGA_A7- | 3 | 2215.118171 | 310.8962209 | 2215.116185 | 739.38 | 15.77291244 | 0.003996004 | BRCA |
| of peptide N- | AOCE_BH- | | | | | | | | |
| term@0[144. | AOCO_A2- | | | | | | | | |
| 1021]; | AOYC_117 | | | | | | | | |
| iTRAQ | C_W_BI_2 | | | | | | | | |
| 4-plex of | 0130524_ | | | | | | | | |
| Y@2[144.1021] | H- | | | | | | | | |
| | PM_f06: | | | | | | | | |
| | 18392: | | | | | | | | |
| | 3 | | | | | | | | |
| iTRAQ 4-plex | TCGA_A8- | 3 | 2247.128171 | 319.8597904 | 2247.106015 | 750.05 | 15.75423476 | 0.002997003 | BRCA |
| of peptide N- | A09G_C8- | | | | | | | | |
| term@0 | A131_C8- | | | | | | | | |
| [144.1021]; | A134_117 | | | | | | | | |
| Oxidation of | C_W_BI_2 | | | | | | | | |
| M@1 | 0131011_ | | | | | | | | |
| [15.9949]; | H- | | | | | | | | |
| iTRAQ 4- | PM_f09: | | | | | | | | |
| plex of | 24324: | | | | | | | | |
| Y@8 | 3 | | | | | | | | |
| [144.1021]; | | | | | | | | | |
| Oxidation | | | | | | | | | |
| of | | | | | | | | | |
| M@11[15.99 | | | | | | | | | |
| 49] | | | | | | | | | |
| iTRAQ 4-plex | CPTAC_2 | 2 | 2359.205447 | 5.425831466 | 2359.218248 | 1180.61 | 14.64410275 | 9.99E−04 | BRCA |
| of peptide N- | 63d3f- | | | | | | | | |
| term@0 | I_blcdb9- | | | | | | | | |
| [144.1021]; | I_c4155b- | | | | | | | | |
| iTRAQ | C_117C_ | | | | | | | | |
| 4-plex of | W_BI_201 | | | | | | | | |
| Y@2 | 40417_H- | | | | | | | | |
| [144.1021]; | PM_f01: | | | | | | | | |
| iTRAQ 4- | 30099: | | | | | | | | |
| plex of | 2 | | | | | | | | |
| Y@8[144.1021] | | | | | | | | | |
| iTRAQ 4-plex | TCGA_AR | 3 | 2375.228171 | 316.3186721 | 2375.213162 | 792.75 | 14.63775209 | 0.002997003 | BRCA |
| of peptide N- | - | | | | | | | | |
| term@0[144. | AOTV_C8- | | | | | | | | |
| 1021]; | A12Z_AO- | | | | | | | | |
| Oxidation of | AOJJ_117 | | | | | | | | |
| M@1 | C_W_BI_2 | | | | | | | | |
| [15.9949]; | 0130730_ | | | | | | | | |
| iTRAQ 4- | H- | | | | | | | | |
| plex of | PM_f15: | | | | | | | | |
| Y@2 | 37695: | | | | | | | | |
| [144.1021]; | 3 | | | | | | | | |
| İTRAQ 4- | | | | | | | | | |
| plex of | | | | | | | | | |
| Y@8 | | | | | | | | | |
| [144.1021] | | | | | | | | | |
| iTRAQ 4-plex | CPTAC_2 | 3 | 2375.228171 | 316.3186721 | 2375.213162 | 792.75 | 14.62779283 | 0.002997003 | BRCA |
| of peptide N- | 63d3f- | | | | | | | | |
| term@0[144. | _blcdb9- | | | | | | | | |
| 1021]; | I_c4155b- | | | | | | | | |
| Oxidation of | C_117C_ | | | | | | | | |
| M@1 | W_BI_ | | | | | | | | |
| [15.9949]; | 201898: | | | | | | | | |
| iTRAQ 4- | 340417_ | | | | | | | | |
| plex of Y@2 | H- | | | | | | | | |
| [144.1021]; | PM_f14: | | | | | | | | |
| iTRAQ 4- | 36 | | | | | | | | |
| plex of | | | | | | | | | |
| Y@8 | | | | | | | | | |
| [144.1021] | | | | | | | | | |
| iTRAQ 4-plex | TCGA_D8 | 3 | 2375.228171 | 316.3186721 | 2375.213162 | 792.75 | 14.60152126 | 9.99E−04 | BRCA |
| of peptide N- | -A13Y_A8- | | | | | | | | |
| term@0[144. | A076_AO- | | | | | | | | |
| 1021]; | A126_117 | | | | | | | | |
| Oxidation of | C_W_BI_2 | | | | | | | | |
| M@1 | 0130617_ | | | | | | | | |
| [15.9949]; | H- | | | | | | | | |
| iTRAQ 4- | PM_f15: | | | | | | | | |
| plex of | 36800: | | | | | | | | |
| Y@2 | 3 | | | | | | | | |
| [144.1021]; | | | | | | | | | |
| İTRAQ 4- | | | | | | | | | |
| plex of | | | | | | | | | |
| Y@8 | | | | | | | | | |
| [144.1021] | | | | | | | | | |
| iTRAQ 4-plex | TCGA_A7- | 3 | 2375.228171 | 316.3186721 | 2375.213162 | 792.75 | 14.55836167 | 9.99E−04 | BRCA |
| of peptide N- | AOCJ_AO- | | | | | | | | |
| term@0[144. | A12F_A2- | | | | | | | | |
| 1021]; | AOYL_117 | | | | | | | | |
| Oxidation of | C_W_BI_2 | | | | | | | | |
| M@1 | 0130805_ | | | | | | | | |
| [15.9949]; | H-PM_f15: | | | | | | | | |
| iTRAQ 4- | 37121: | | | | | | | | |
| plex of | 3 | | | | | | | | |
| Y@2 | | | | | | | | | |
| [144.1021]; | | | | | | | | | |
| iTRAQ 4- | | | | | | | | | |
| plex of | | | | | | | | | |
| Y@8 | | | | | | | | | |
| [144.1021] | | | | | | | | | |
| iTRAQ 4-plex | TCGA_AR | 3 | 2087.018171 | 314.3761356 | 2087.009038 | 696.68 | 14.52796333 | 0.001998002 | BRCA |
| of peptide N- | - | | | | | | | | |
| term@0 | AOTT_AR- | | | | | | | | |
| [144.1021]; | A1AQ_AO | | | | | | | | |
| Oxidation of | A12B_117 | | | | | | | | |
| M@1 | C_W_BI_2 | | | | | | | | |
| [15.9949] | 0131022_ | | | | | | | | |
| | H- | | | | | | | | |
| | PM_f20: | | | | | | | | |
| | 34431: | | | | | | | | |
| | 3 | | | | | | | | |
| iTRAQ 4-plex | TCGA_AR | 4 | 2087.010894 | 310.889583 | 2087.009038 | 522.76 | 14.51808309 | 0.005994006 | BRCA |
| of peptide N- | - | | | | | | | | |
| term@0 | AOTR_AO- | | | | | | | | |
| [144.1021]; | A03O_BH- | | | | | | | | |
| Oxidation of | A18R_117 | | | | | | | | |
| M@11 | C_W_BI_2 | | | | | | | | |
| [15.9949] | 0130825_ | | | | | | | | |
| | H- | | | | | | | | |
| | PM_f03: | | | | | | | | |
| | 6191: | | | | | | | | |
| | 4 | | | | | | | | |
| iTRAQ 4-plex | TCGA_AO | 4 | 2231.130894 | 318.8718787 | 2231.1111 | 558.79 | 14.42712204 | 0.00999001 | BRCA |
| of peptide N- | - | | | | | | | | |
| term@0 | AOJM_C8- | | | | | | | | |
| [144.1021]; | A12V_A8- | | | | | | | | |
| iTRAQ | A08G_117 | | | | | | | | |
| 4-plex of | C_W_BI_2 | | | | | | | | |
| Y@2 | 0130927_ | | | | | | | | |
| [144.1021]; | H- | | | | | | | | |
| Oxidation | PM_124: | | | | | | | | |
| of | 6547: | | | | | | | | |
| M@11[15.9949] | 4 | | | | | | | | |
| iTRAQ 4-plex | TCGA_AO | 4 | 2231.130894 | 318.8718787 | 2231.1111 | 558.79 | 14.41283587 | 0.005994006 | BRCA |
| of peptide N- | - | | | | | | | | |
| term@0[144. | A12D_AN- | | | | | | | | |
| 1021]; | A04A_BH- | | | | | | | | |
| iTRAQ | AOAV_117 | | | | | | | | |
| 4-plex of | C_W_BI_2 | | | | | | | | |
| Y@2 | 0130310_ | | | | | | | | |
| [144.1021]; | H- | | | | | | | | |
| Oxidation | PM_f24: | | | | | | | | |
| of | 5640: | | | | | | | | |
| M@11 | 4 | | | | | | | | |
| [15.9949] | | | | | | | | | |
| iTRAQ 4-plex | TCGA_A8- | 3 | 2087.018171 | 314.3761356 | 2087.009038 | 696.68 | 14.41085006 | 0.006993007 | BRCA |
| of peptide N- | A06Z_A2- | | | | | | | | |
| term@0 | AOD1_A2- | | | | | | | | |
| [144.1021]; | AOCM_11 | | | | | | | | |
| Oxidation of | 7C_W_BI_ | | | | | | | | |
| M@1 | 20130401_ | | | | | | | | |
| [15.9949] | H- | | | | | | | | |
| | JQ_f19: | | | | | | | | |
| | 32145: | | | | | | | | |
| | 3 | | | | | | | | |
| iTRAQ 4-plex | TCGA_C8 | 4 | 2231.130894 | 318.8718787 | 2231.1111 | 558.79 | 14.40291217 | 0.005994006 | BRCA |
| of peptide N- | -A138_E2- | | | | | | | | |
| term@0[144. | A154_BH- | | | | | | | | |
| 1021]; | AOBZ_117 | | | | | | | | |
| iTRAQ | C_W_BI_2 | | | | | | | | |
| 4-plex of | 0130225_ | | | | | | | | |
| Y@2 | H- | | | | | | | | |
| [144.1021]; | PM_f24: | | | | | | | | |
| Oxidation | 6661: | | | | | | | | |
| of | 4 | | | | | | | | |
| M@11 | | | | | | | | | |
| [15.9949] | | | | | | | | | |
| iTRAQ 4-plex | TCGA_AR | 3 | 2087.018171 | 314.3761356 | 2087.009038 | 696.68 | 14.21999861 | 0.004995005 | BRCA |
| of peptide N- | -AOTT_ | | | | | | | | |
| term@0 | AR- | | | | | | | | |
| [144.1021]; | A1AQ_AO- | | | | | | | | |
| Oxidation of | A12B_117 | | | | | | | | |
| M@11 | C_W_BI_2 | | | | | | | | |
| [15.9949] | 0131022_ | | | | | | | | |
| | H- | | | | | | | | |
| | PM_f20: | | | | | | | | |
| | 34553: | | | | | | | | |
| | 3 | | | | | | | | |
| iTRAQ 4-plex | TCGA_AR | 2 | 2087.025447 | 317.8626882 | 2087.009038 | 1044.52 | 14.13564819 | 0.001998002 | BRCA |
| of peptide N- | -_AOTT_AR- | | | | | | | | |
| term@0 | A1AQ_AO- | | | | | | | | |
| [144.1021]; | A12B_117 | | | | | | | | |
| Oxidation of | C_W_BI_2 | | | | | | | | |
| M@1[15.9949] | 0131022_ | | | | | | | | |
| | H- | | | | | | | | |
| | PM_f20: | | | | | | | | |
| | 34469: | | | | | | | | |
| | 2 | | | | | | | | |
| iTRAQ 4-plex | TCGA_ | 2 | 2087.025447 | 317.8626882 | 2087.009038 | 104452 | 14.10986902 | 0.002997003 | BRCA |
| of peptide N- | AN- | | | | | | | | |
| term@0 | AOFL_BH- | | | | | | | | |
| [144.1021]; | AODG_AN | | | | | | | | |
| Oxidation of | AOAS_117 | | | | | | | | |
| M@1 | C_W_BI_2 | | | | | | | | |
| [15.9949] | 0130726_ | | | | | | | | |
| | H- | | | | | | | | |
| | PM_f19: | | | | | | | | |
| | 34842: | | | | | | | | |
| | 2 | | | | | | | | |
| iTRAQ 4-plex | TCGA_BH | 2 | 2087.025447 | 317.8626882 | 2087.009038 | 1044.52 | 14.10125275 | 0.002997003 | BRCA |
| of peptide N- | - | | | | | | | | |
| term@0 | AOEE_AO- | | | | | | | | |
| [144.1021]; | AOJ9_BH- | | | | | | | | |
| Oxidation of | AOE0_117 | | | | | | | | |
| M@1 | C_W_BI_2 | | | | | | | | |
| [15.9949] | 0130412_ | | | | | | | | |
| | H- | | | | | | | | |
| | PM_f20: | | | | | | | | |
| | 37258: | | | | | | | | |
| | 2 | | | | | | | | |
| iTRAQ 4-plex | TCGA_AO | 3 | 2215.118171 | 310.8962209 | 2215.116185 | 739.38 | 14.07807459 | 0.006993007 | BRCA |
| of peptide N- | - | | | | | | | | |
| term@0 | A12D_AN- | | | | | | | | |
| [144.1021]; | A04A_BH- | | | | | | | | |
| iTRAQ | AOAV_117 | | | | | | | | |
| 4-plex of | C_W_BI_2 | | | | | | | | |
| Y@2 | 0130416_ | | | | | | | | |
| [144.1021] | H- | | | | | | | | |
| | PM_f14: | | | | | | | | |
| | 41266: | | | | | | | | |
| | 3 | | | | | | | | |
| iTRAQ 4-plex | TCGA_AO | 2 | 2087.025447 | 317.8626882 | 2087.009038 | 1044.52 | 14.0677017 | 0.001998002 | BRCA |
| of peptide N- | -AOJE_A2- | | | | | | | | |
| term@0 | AOT2_AN- | | | | | | | | |
| [144.1021]; | AOAJ_117 | | | | | | | | |
| Oxidation of | C_W_BI_2 | | | | | | | | |
| M@1[15.9949] | 0130802_ | | | | | | | | |
| | H- | | | | | | | | |
| | PM_f19: | | | | | | | | |
| | 38410: | | | | | | | | |
| | 2 | | | | | | | | |
| iTRAQ 4-plex | TCGA_D8 | 2 | 2087.025447 | 317.8626882 | 2087.009038 | 1044.52 | 14.0670005 | 9.99E−04 | BRCA |
| of peptide N- | -A13Y_A8- | | | | | | | | |
| term@0 | A076_AO- | | | | | | | | |
| [144.1021]; | A126_117 | | | | | | | | |
| Oxidation of | C_W_BI_2 | | | | | | | | |
| M@1 | 0130617_ | | | | | | | | |
| [15.9949] | H- | | | | | | | | |
| | PM_f19: | | | | | | | | |
| | 37437: | | | | | | | | |
| | 2 | | | | | | | | |
| iTRAQ 4-plex | TCGA_C8 | 2 | 2087.025447 | 317.8626882 | 2087.009038 | 1044.52 | 14.0397655 | 9.99E−04 | BRCA |
| of peptide N- | - | | | | | | | | |
| term@0 | A12P_BH- | | | | | | | | |
| [144.1021]; | AOC1_A2- | | | | | | | | |
| Oxidation of | AOEY_117 | | | | | | | | |
| M@1 | C_W_BI_2 | | | | | | | | |
| [15.9949] | 0130622_ | | | | | | | | |
| | H- | | | | | | | | |
| | PM_f18: | | | | | | | | |
| | 34817: | | | | | | | | |
| | 2 | | | | | | | | |
| iTRAQ 4-plex | TCGA_A2- | 2 | 2087.025447 | 317.8626882 | 2087.009038 | 1044.52 | 14.01121141 | 9.99E−04 | BRCA |
| of peptide N- | AOT7_C8- | | | | | | | | |
| term@0 | A12Q_A8- | | | | | | | | |
| [144.1021]; | A079_117 | | | | | | | | |
| Oxidation of | C_W_BI_2 | | | | | | | | |
| M@1 | 0130820_ | | | | | | | | |
| [15.9949] | H- | | | | | | | | |
| | PM_f19: | | | | | | | | |
| | 37861: | | | | | | | | |
| | 2 | | | | | | | | |
| iTRAQ 4-plex | TCGA_A8- | 3 | 2087.018171 | 314.3761356 | 2087.009038 | 696.68 | 14.00858049 | 0.008991009 | BRCA |
| of peptide N- | A09G_C8- | | | | | | | | |
| term@0 | A131_C8- | | | | | | | | |
| [144.1021]; | A134_117 | | | | | | | | |
| Oxidation of | C_W_BI_2 | | | | | | | | |
| M@11 | 0131011_ | | | | | | | | |
| [15.9949] | H- | | | | | | | | |
| | PM_f19: | | | | | | | | |
| | 35762: | | | | | | | | |
| | 3 | | | | | | | | |
| iTRAQ 4-plex | TCGA_A2- | 3 | 2087.018171 | 314.3761356 | 2087.009038 | 696.68 | 13.98394207 | 0.003996004 | BRCA |
| of peptide N- | AOYM_BH | | | | | | | | |
| term@0[144. | - | | | | | | | | |
| 1021]; | AOC7_A2- | | | | | | | | |
| Oxidation of | AOSX_117 | | | | | | | | |
| M@11[15.9949] | C_W_BI_2 | | | | | | | | |
| | 0131025_ | | | | | | | | |
| | H- | | | | | | | | |
| | PM_f19: | | | | | | | | |
| | 28223: | | | | | | | | |
| | 3 | | | | | | | | |
| iTRAQ 4-plex | TCGA_E2- | 4 | 2231.130894 | 318.8718787 | 2231.1111 | 558.79 | 13.93947078 | 0.004995005 | BRCA |
| of peptide N- | A10A_BH- | | | | | | | | |
| term@0 | A18Q_C8- | | | | | | | | |
| [144.1021]; | A130_117 | | | | | | | | |
| iTRAQ | C_W_BI_2 | | | | | | | | |
| 4-plex of | 0130222_ | | | | | | | | |
| Y@2 | H- | | | | | | | | |
| [144.1021]; | PM_f24: | | | | | | | | |
| Oxidation | 6726: | | | | | | | | |
| of | 4 | | | | | | | | |
| M@11 | | | | | | | | | |
| [15.9949] | | | | | | | | | |
| iTRAQ 4-plex | TCGA_AO | 3 | 2087.018171 | 314.3761356 | 2087.009038 | 696.68 | 13.90022959 | 0.004995005 | BRCA |
| of peptide N- | - | | | | | | | | |
| term@0 | A12D_AN- | | | | | | | | |
| [144.1021]; | A04A_BH- | | | | | | | | |
| Oxidation | AOAV_117 | | | | | | | | |
| of | C_W_BI_2 | | | | | | | | |
| M@1 | 0130416_ | | | | | | | | |
| [15.9949] | H- | | | | | | | | |
| | PM_f19: | | | | | | | | |
| | 37719: | | | | | | | | |
| | 3 | | | | | | | | |
| iTRAQ 4-plex | TCGA_A2- | 2 | 2087.025447 | 317.8626882 | 2087.009038 | 1044.52 | 13.89656997 | 9.99E−04 | BRCA |
| of peptide N- | AODO_BH- | | | | | | | | |
| term@0 | AOHK_C8- | | | | | | | | |
| [144.1021]; | A12T_117 | | | | | | | | |
| Oxidation | C_W_BI_2 | | | | | | | | |
| of | 0130326_ | | | | | | | | |
| M@1 | H- | | | | | | | | |
| [15.9949] | JQ_f20: | | | | | | | | |
| | 32142: | | | | | | | | |
| | 2 | | | | | | | | |
| iTRAQ 4-plex | TCGA_C8 | 2 | 2087.025447 | 317.8626882 | 2087.009038 | 104452 | 13.83473588 | 0.002997003 | BRCA |
| of peptide N- | -A138_E2- | | | | | | | | |
| term@0 | A154_BH- | | | | | | | | |
| [144.1021]; | AOBZ_117 | | | | | | | | |
| Oxidation | C_W_BI_2 | | | | | | | | |
| of | 0130225_ | | | | | | | | |
| M@1 | H- | | | | | | | | |
| [15.9949] | PM_f20: | | | | | | | | |
| | 36777: | | | | | | | | |
| | 2 | | | | | | | | |
| iTRAQ 4-plex | TCGA_E2- | 4 | 2231.130894 | 318.8718787 | 2231.1111 | 558.79 | 13.77205915 | 0.003996004 | BRCA |
| of peptide N- | A10A_BH- | | | | | | | | |
| term@0 | A18Q_C8- | | | | | | | | |
| [144.1021]; | A130_117 | | | | | | | | |
| iTRAQ | C_W_BI_2 | | | | | | | | |
| 4-plex of | 0130222_ | | | | | | | | |
| Y@2 | H- | | | | | | | | |
| [144.1021]; | PM_f01: | | | | | | | | |
| Oxidation | 6326: | | | | | | | | |
| of | 4 | | | | | | | | |
| M@11[15.99 | | | | | | | | | |
| 49] | | | | | | | | | |
| iTRAQ 4-plex | TCGA_A8- | 4 | 2215.130894 | 316.6401784 | 2215.116185 | 554.79 | 13.65481715 | 0.004995005 | BRCA |
| of peptide N- | A091_C8- | | | | | | | | |
| term@0[144. | A12L_A2- | | | | | | | | |
| 1021]; | AOEX_117 | | | | | | | | |
| iTRAQ | C_W_BI_2 | | | | | | | | |
| 4-plex of | 0130302_ | | | | | | | | |
| Y@2[144.1021] | H- | | | | | | | | |
| | PM_f24: | | | | | | | | |
| | 6807: | | | | | | | | |
| | 4 | | | | | | | | |
| iTRAQ 4-plex | TCGA_A2- | 4 | 2087.010894 | 310.889583 | 2087.009038 | 522.76 | 13.64308712 | 0.001998002 | BRCA |
| of peptide N- | AOT7_C8- | | | | | | | | |
| term@0[144. | A12Q_A8- | | | | | | | | |
| 1021]; | A079_117 | | | | | | | | |
| Oxidation | C_W_BI_2 | | | | | | | | |
| of | 0130820_ | | | | | | | | |
| M@11[15.9949] | H- | | | | | | | | |
| | PM_f03: | | | | | | | | |
| | 6512: | | | | | | | | |
| | 4 | | | | | | | | |
| iTRAQ 4-plex | TCGA_ | 2 | 2087.005447 | 1.720403937 | 2087.009038 | 1044.51 | 13.54023999 | 0.002997003 | BRCA |
| of peptide N- | AO- | | | | | | | | |
| term@0 | A12D_AN- | | | | | | | | |
| [144.1021]; | A04A_BH- | | | | | | | | |
| Oxidation | AOAV_117 | | | | | | | | |
| of | C_W_BI_2 | | | | | | | | |
| M@1 | 0130310_ | | | | | | | | |
| [15.9949] | H- | | | | | | | | |
| | PM_f20: | | | | | | | | |
| | 34519: | | | | | | | | |
| | 2 | | | | | | | | |
| iTRAQ 4-plex | TCGA_AO | 2 | 2087.025447 | 317.8626882 | 2087.009038 | 1044.52 | 13.51017933 | 9.99E−04 | BRCA |
| of peptide N- | - | | | | | | | | |
| term@0 | A12D_AN- | | | | | | | | |
| [144.1021]; | A04A_BH- | | | | | | | | |
| Oxidation | AOAV_117 | | | | | | | | |
| of | C_W_BI_2 | | | | | | | | |
| M@1 | 0130310_ | | | | | | | | |
| [15.9949] | H- | | | | | | | | |
| | PM_f19: | | | | | | | | |
| | 35293: | | | | | | | | |
| | 2 | | | | | | | | |
| iTRAQ 4-plex | TCGA_E2- | 2 | 2087.005447 | 1.720403937 | 2087.009038 | 1044.51 | 13.49889843 | 0.001998002 | BRCA |
| of peptide N- | A10A_BH- | | | | | | | | |
| term@0 | A18Q_C8- | | | | | | | | |
| [144.1021]; | A130_117 | | | | | | | | |
| Oxidation | C_W_BI_2 | | | | | | | | |
| of | 0130222_ | | | | | | | | |
| M@1 | H- | | | | | | | | |
| [15.9949] | PM_f19: | | | | | | | | |
| | 38508: | | | | | | | | |
| | 2 | | | | | | | | |
| iTRAQ 4-plex | TCGA_ | 3 | 2231.108171 | 1.312971898 | 2231.1111 | 744.71 | 13.49830934 | 0.008991009 | BRCA |
| of peptide N- | AO- | | | | | | | | |
| term@0 | A12D_AN- | | | | | | | | |
| [144.1021]; | A04A_BH- | | | | | | | | |
| Oxidation | AOAV_117 | | | | | | | | |
| of | C_W_BI_2 | | | | | | | | |
| M@1 | 0130416_ | | | | | | | | |
| [15.9949]; | H- | | | | | | | | |
| iTRAQ 4- | PM_f23: | | | | | | | | |
| plex of | 16587: | | | | | | | | |
| Y@8 | 3 | | | | | | | | |
| [144.1021] | | | | | | | | | |
| iTRAQ 4-plex | TCGA_AO | 3 | 2359.238171 | 318.444667 | 2359.218248 | 787.42 | 13.48419625 | 0.007992008 | BRCA |
| of peptide N- | -A12E_A8- | | | | | | | | |
| term@0 | A06N_A2- | | | | | | | | |
| [144.1021]; | AOT1_117 | | | | | | | | |
| iTRAQ | C_W_ | | | | | | | | |
| 4-plex of | BI_2 | | | | | | | | |
| Y@2 | 0130909_ | | | | | | | | |
| [144.1021]; | H- | | | | | | | | |
| iTRAQ 4- | PM_f05: | | | | | | | | |
| plex of | 37703: | | | | | | | | |
| Y@8[144.1021] | 3 | | | | | | | | |
| iTRAQ 4-plex | TCGA_A8- | 3 | 2215.118171 | 310.8962209 | 2215.116185 | 739.38 | 13.42021779 | 0.007992008 | BRCA |
| of peptide N- | A09G_C8- | | | | | | | | |
| term@0 | A131_C8- | | | | | | | | |
| [144.1021]; | A134_117 | | | | | | | | |
| iTRAQ | C_W_BI_2 | | | | | | | | |
| 4-plex of | 0131011_ | | | | | | | | |
| Y@2 | H- | | | | | | | | |
| [144.1021] | PM_f24: | | | | | | | | |
| | 7519: | | | | | | | | |
| | 3 | | | | | | | | |
| iTRAQ 4-plex | TCGA_AO | 3 | 2359.208171 | 4.271409576 | 2359.218248 | 787.41 | 13.1260299 | 0.008991009 | BRCA |
| of peptide N- | - | | | | | | | | |
| term@0 | A12D_AN- | | | | | | | | |
| [144.1021]; | A04A_BH- | | | | | | | | |
| iTRAQ | AOAV_117 | | | | | | | | |
| 4-plex of | C_W_BI_2 | | | | | | | | |
| Y@2 | 0130416_ | | | | | | | | |
| [144.1021]; | H- | | | | | | | | |
| iTRAQ 4- | PM_f22: | | | | | | | | |
| plex of | 29730: | | | | | | | | |
| Y@8 | 3 | | | | | | | | |
| [144.1021] | | | | | | | | | |
| iTRAQ 4-plex | TCGA_AR | 3 | 2359.208171 | 4.271409576 | 2359.218248 | 787.41 | 12.98000352 | 0.00999001 | BRCA |
| of peptide N- | - | | | | | | | | |
| term@0 | AOTY_AR- | | | | | | | | |
| [144.1021]; | AOU4_BH- | | | | | | | | |
| iTRAQ | AOHP_117 | | | | | | | | |
| 4-plex of | C_W_BI_2 | | | | | | | | |
| Y@2 | 0130408_ | | | | | | | | |
| [144.1021]; | H- | | | | | | | | |
| iTRAQ 4- | JQ_f17: | | | | | | | | |
| plex of | 25054: | | | | | | | | |
| Y@8 | 3 | | | | | | | | |
| [144.1021] | | | | | | | | | |
| iTRAQ 4-plex | TCGA_C8 | 3 | 2215.118171 | 310.8962209 | 2215.116185 | 739.38 | 12.94538391 | 0.006993007 | BRCA |
| of | -A12P_ | | | | | | | | |
| peptide | BH- | | | | | | | | |
| N-term@0 | AOC1_A2- | | | | | | | | |
| [144.1021]; | AOEY_117 | | | | | | | | |
| iTRAQ | C_W_BI_2 | | | | | | | | |
| 4-plex of | 0130622_ | | | | | | | | |
| Y@2 | H- | | | | | | | | |
| [144.1021] | PM_f24: | | | | | | | | |
| | 6060: | | | | | | | | |
| | 3 | | | | | | | | |
| iTRAQ 4-plex | TCGA_AR | 4 | 2231.130894 | −8.871878685 | 2231.1111 | 558.79 | 12.80852006 | 0.007992008 | BRCA |
| of peptide N- | -AOTY_ | | | | | | | | |
| term@0 | AR- | | | | | | | | |
| [144.1021]; | AOU4_BH- | | | | | | | | |
| iTRAQ | AOHP_117 | | | | | | | | |
| 4-plex of | C_W_BI_2 | | | | | | | | |
| Y@2 | 0130408_ | | | | | | | | |
| [144.1021]; | H- | | | | | | | | |
| Oxidation | JQ_f24: | | | | | | | | |
| of | 6317: | | | | | | | | |
| M@11 | 4 | | | | | | | | |
| [15.9949] | | | | | | | | | |
| iTRAQ 4-plex | TCGA_BH | 2 | 2247.085447 | 9.152901016 | 2247.106015 | 1124.55 | 12.8004864 | 0.002997003 | BRCA |
| of peptide N- | - | | | | | | | | |
| term@0 | A18U_A2- | | | | | | | | |
| [144.1021]; | AOYI_A2- | | | | | | | | |
| Oxidation of | AOEQ_117 | | | | | | | | |
| M@1 | C_W_BI_2 | | | | | | | | |
| [15.9949]; | 0130405 | | | | | | | | |
| iTRAQ 4- | H- | | | | | | | | |
| plex of | JQ_f14: | | | | | | | | |
| Y@8 | 12231: | | | | | | | | |
| [144.1021]; | 2 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_C8 | 3 | 2359.208171 | 4.271409576 | 2359.21824 8 | 787.41 | 12.69108278 | 0.008991009 | BRCA |
| of peptide N- | -A138_E2- | | | | | | | | |
| term@0 | A154_BH- | | | | | | | | |
| [144.1021]; | AOBZ_117 | | | | | | | | |
| İTRAQ | C_W_BI_ | | | | | | | | |
| 4-plex of | 20130225_ | | | | | | | | |
| Y@2 | H- | | | | | | | | |
| [144.1021]; | PM_f17: | | | | | | | | |
| iTRAQ 4- | 30187: | | | | | | | | |
| plex of | 3 | | | | | | | | |
| Y@8 | | | | | | | | | |
| [144.1021] | | | | | | | | | |
| iTRAQ 4-plex | TCGA_AR | 3 | 2215.118171 | −0.896220883 | 2215.116185 | 739.38 | 12.6695597 | 0.005994006 | BRCA |
| of peptide N- | -AOTV_ | | | | | | | | |
| term@0 | C8- | | | | | | | | |
| [144.1021]; | A12Z_AO- | | | | | | | | |
| iTRAQ | AOJJ_117 | | | | | | | | |
| 4-plex of | C_W_BI_2 | | | | | | | | |
| Y@8 | 0130730_ | | | | | | | | |
| [144.1021] | H- | | | | | | | | |
| | PM_f21: | | | | | | | | |
| | 38288: | | | | | | | | |
| | 3 | | | | | | | | |
| iTRAQ 4-plex | TCGA_A2- | 2 | 2231.125447 | −6.43046446 | 2231.1111 | 1116.57 | 12.28516607 | 0.002997003 | BRCA |
| of peptide N- | AOT7_C8- | | | | | | | | |
| term@0 | A12Q_A8- | | | | | | | | |
| [144.1021]; | A079_117 | | | | | | | | |
| Oxidation of | C_W_BI_2 | | | | | | | | |
| M@1 | 0130820_ | | | | | | | | |
| [15.9949]; | H- | | | | | | | | |
| iTRAQ 4- | PM_f15: | | | | | | | | |
| plex of | 31729: | | | | | | | | |
| Y@8 | 2 | | | | | | | | |
| [144.1021] | | | | | | | | | |
| iTRAQ 4-plex | TCGA_AO | 4 | 2087.010894 | −0.889583025 | 2087.009038 | 522.76 | 12.27878069 | 0.006993007 | BRCA |
| of peptide N- | -AOJC_A8- | | | | | | | | |
| term@0 | A08Z_AR- | | | | | | | | |
| [144.1021]; | AOTX_117 | | | | | | | | |
| Oxidation of | C_W_BI_2 | | | | | | | | |
| M@1 | 0130527_ | | | | | | | | |
| [15.9949] | H- | | | | | | | | |
| | PM_f10: | | | | | | | | |
| | 20350: | | | | | | | | |
| | 4 | | | | | | | | |
| iTRAQ 4-plex | TCGA_A2- | 4 | 2215.130894 | −6.640178366 | 2215.116185 | 554.79 | 12.26434715 | 0.00999001 | BRCA |
| of peptide N- | AODO_BH- | | | | | | | | |
| term@0 | AOHK_C8- | | | | | | | | |
| [144.1021]; | A12T_117 | | | | | | | | |
| iTRAQ | C_W_BI_2 | | | | | | | | |
| 4-plex of | 0130326_ | | | | | | | | |
| Y@2 | H- | | | | | | | | |
| [144.1021] | JQ_f24: | | | | | | | | |
| | 6745: | | | | | | | | |
| | 4 | | | | | | | | |
| iTRAQ 4-plex | TCGA_AO | 4 | 2071.010894 | 1.559049063 | 2071.014123 | 518.76 | 12.19226041 | 0.006993007 | BRCA |
| of peptide N- | -AOJE_A2- | | | | | | | | |
| term@0 | AOT2_AN- | | | | | | | | |
| [144.1021] | AOAJ_117 | | | | | | | | |
| | C_W_BI_2 | | | | | | | | |
| | 0130802_ | | | | | | | | |
| | H- | | | | | | | | |
| | PM_f16: | | | | | | | | |
| | 5866: | | | | | | | | |
| | 4 | | | | | | | | |
| iTRAQ 4-plex | TCGA_ | 3 | 2215.118171 | −0.896220883 | 2215.116185 | 739.38 | 12.14428524 | 0.003996004 | BRCA |
| of peptide N- | BH- | | | | | | | | |
| term@0[144. | AOEE_AO- | | | | | | | | |
| 1021]; | AOJ9_BH- | | | | | | | | |
| iTRAQ | AOEO_117 | | | | | | | | |
| 4-plex of | C_W_BI_2 | | | | | | | | |
| Y@2 | 0130412_ | | | | | | | | |
| [144.1021] | H- | | | | | | | | |
| | PM_f01: | | | | | | | | |
| | 6625: | | | | | | | | |
| | 3 | | | | | | | | |

Peptide: MYQPIQTYPWMNLSRR (SEQ ID NO: 20)

| | | | | | | | | | |
|---|---|---|---|---|---|---|---|---|---|
| TMT 10-plex | 13CPTAC_ | 3 | 2328.190024 | −8.16319948 | 2328.171018 | 777.07061 | 20.67328769 | 10.008991009 | UCEC |
| of peptide N- | UCEC_W_ | | | | | | | | |
| term@0[229. | PNNL_20 | | | | | | | | |
| 1629]; | 180503_ | | | | | | | | |
| Oxidation of | B | | | | | | | | |
| M@11[15.9949] | 4S1_f06: | | | | | | | | |
| | 39892:3 | | | | | | | | |
| TMT 10-plex | 14CPTAC_ | 4 | 2312.196968 | −9.023823302 | 2312.176104 | 579.0565186 | 14.62543433 | 0.003996004 | UCEC |
| of peptide N- | UCEC_W_ | | | | | | | | |
| term@0 | PNNL_20 | | | | | | | | |
| [229.1629] | 180503_B | | | | | | | | |
| | 4S2_f24: | | | | | | | | |
| | 3 | | | | | | | | |
| | 3490: | | | | | | | | |
| | 4 | | | | | | | | |
| TMT 10-plex | 23CPTAC_ | 3 | 2312.175802 | 0.130287393 | 2312.176104 | 771.7325439 | 18.72747628 | 0.008991009 | LUAD |
| of peptide N- | LUAD_W_ | | | | | | | | |
| term@0[229. | BI_20180 | | | | | | | | |
| 1629] | 822_KR_f | | | | | | | | |
| | 08: | | | | | | | | |
| | 11639: | | | | | | | | |
| | 3 | | | | | | | | |
| TMT 10-plex | 20CPTAC_ | 4 | 2344.145211 | 8.839981302 | 2344.165933 | 587.0435791 | 17.55243803 | 0.008991009 | LUAD |
| of peptide N- | LUAD_W_ | | | | | | | | |
| term@0 | BI_20180 | | | | | | | | |
| [229.1629]; | 809_KR_f | | | | | | | | |
| Oxidation of | 01: | | | | | | | | |
| M@1 | 42299: | | | | | | | | |
| [15.9949]; | 4 | | | | | | | | |
| Oxidation | | | | | | | | | |
| of | | | | | | | | | |
| M@11 | | | | | | | | | |
| [15.9949] | | | | | | | | | |
| TMT 10-plex | 18CPTAC_ | 3 | 2328.191672 | −8.87102945 | 2328.171018 | 777.071167 | 17.32700385 | 0.007992008 | LUAD |
| of peptide N- | LUAD_W_ | | | | | | | | |
| term@0 | BI_20180 | | | | | | | | |
| [229.1629]; | 726_KL_f0 | | | | | | | | |
| Oxidation of | 8: | | | | | | | | |
| M@11 | 38624: | | | | | | | | |
| [15.9949] | 3 | | | | | | | | |
| TMT 10-plex | 04CPTAC_ | 3 | 2328.153952 | 7.330412054 | 2328.171018 | 777.0585938 | 15.27424191 | 9.99E−04 | ILUAD |
| of peptide N- | LUAD_W_ | | | | | | | | |
| term@0 | BI_20180 | | | | | | | | |
| [229.1629]; | 524_KR_f | | | | | | | | |
| Oxidation | 19: | | | | | | | | |
| of | 37406:3 | | | | | | | | |
| M@11 | | | | | | | | | |
| [15.9949] | | | | | | | | | |
| TMT 10-plex | 04CPTAC_ | 3 | 2328.185629 | −6.275652897 | 2328.171018 | 777.0691528 | 13.53696176 | 0.003996004 | LUAD |
| of peptide N- | LUAD_W_ | | | | | | | | |
| term@0 | BI_20180 | | | | | | | | |
| [229.1629]; | 524_KR_f | | | | | | | | |
| Oxidation | 07: | | | | | | | | |
| of | 21798: | | | | | | | | |
| M@11 | 3 | | | | | | | | |
| [15.9949] | | | | | | | | | |
| iTRAQ 4-plex | TCGA_C8 | 3 | 2243.108171 | 0.881806441 | 2243.110149 | 748.71 | 24.875 66584 | 9.99E−04 | BRCA |
| of peptide N- | -A138_E2- | | | | | | | | |
| term@0 | A154_BH- | | | | | | | | |
| [144.1021]; | AOBZ_117 | | | | | | | | |
| Oxidation of | C_W_BI_2 | | | | | | | | |
| M@1 | 0130225_ | | | | | | | | |
| [15.9949] | H- | | | | | | | | |
| | PM_f23: | | | | | | | | |
| | 32999: | | | | | | | | |
| | 3 | | | | | | | | |
| iTRAQ 4-plex | TCGA_A8- | 3 | 2243.108171 | 0.881806441 | 2243.110149 | 748.71 | 21.31712585 | 0.003996004 | BRCA |
| of peptide N- | A091_C8- | | | | | | | | |
| term@0[144. | A12L_A2- | | | | | | | | |
| 1021]; | AOEX_117 | | | | | | | | |
| Oxidation of | C_W_BI_2 | | | | | | | | |
| M@11 | 0130302_ | | | | | | | | |
| [15.9949] | H- | | | | | | | | |
| | PM_f24: | | | | | | | | |
| | 32350: | | | | | | | | |
| | 3 | | | | | | | | |
| iTRAQ 4-plex | TCGA_ | 3 | 2243.108171 | 0.881806441 | 2243.110149 | 748.71 | 21.3118649 | 0.001998002 | BRCA |
| of peptide N- | AO- | | | | | | | | |
| term@0 | A12D_AN- | | | | | | | | |
| [144.1021]; | A04A_BH- | | | | | | | | |
| Oxidation of | AOAV_117 | | | | | | | | |
| M@11 | C_W_BI_2 | | | | | | | | |
| [15.9949] | 0130310_ | | | | | | | | |
| | H- | | | | | | | | |
| | PM_f23: | | | | | | | | |
| | 31122: | | | | | | | | |
| | 3 | | | | | | | | |
| iTRAQ 4-plex | TCGA_ | 3 | 2403.188171 | 7.887388954 | 2403.207126 | 802.07 | 20.95342244 | 9.99E−04 | BRCA |
| of peptide N- | AO- | | | | | | | | |
| term@0 | AOJM_C8- | | | | | | | | |
| [144.1021]; | A12V_A8- | | | | | | | | |
| Oxidation of | A08G_117 | | | | | | | | |
| M@1 | C_W_BI_2 | | | | | | | | |
| [15.9949]; | 0130927_ | | | | | | | | |
| İTRAQ 4- | H- | | | | | | | | |
| plex of | PM_f03: | | | | | | | | |
| Y@8 | 26958: | | | | | | | | |
| [144.1021]; | 3 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_A7- | 3 | 2387.228171 | −6.685450983 | 2387.212211 | 796.75 | 19.70934688 | 0.005994006 | BRCA |
| of peptide N- | AOCD_C8- | | | | | | | | |
| term@0 | A12W_AN | | | | | | | | |
| [144.1021]; | - | | | | | | | | |
| Oxidation of | AOAL_117 | | | | | | | | |
| M@1 | C_W_BI_2 | | | | | | | | |
| [15.9949]; | 0130913_ | | | | | | | | |
| iTRAQ 4- | H- | | | | | | | | |
| plex of | PM_f14_1 | | | | | | | | |
| Y@2 | 30917083 | | | | | | | | |
| [144.1021] | 211: | | | | | | | | |
| | 25675: | | | | | | | | |
| | 3 | | | | | | | | |
| TRAQ 4-plex | TCGA_BH | 3 | 2403.188171 | 7.887388954 | 2403.207126 | 802.07 | 18.70529112 | 0.001998002 | BRCA |
| of peptide N- | - | | | | | | | | |
| term@0 | A18U_A2- | | | | | | | | |
| [144.1021]; | AOYI_A2- | | | | | | | | |
| Oxidation of | AOEQ_117 | | | | | | | | |
| M@1 | C_W_BI_2 | | | | | | | | |
| [15.9949]; | 0130405_ | | | | | | | | |
| iTRAQ 4- | H- | | | | | | | | |
| plex of | JQ_f03: | | | | | | | | |
| Y@2 | 25191: | | | | | | | | |
| [144.1021]; | 3 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_A8- | 3 | 2243.108171 | 0.881806441 | 2243.110149 | 748.71 | 18.12596444 | 0.002997003 | BRCA |
| of peptide N- | A09G_C8- | | | | | | | | |
| term@0 | A131_C8- | | | | | | | | |
| [144.1021]; | A134_117 | | | | | | | | |
| Oxidation of | C_W_BI_2 | | | | | | | | |
| M@11 | 0131011_ | | | | | | | | |
| [15.9949] | H- | | | | | | | | |
| | PM_f23: | | | | | | | | |
| | 31631: | | | | | | | | |
| | 3 | | | | | | | | |
| iTRAQ 4-plex | TCGA_A2- | 3 | 2243.108171 | 0.881806441 | 2243.110149 | 748.71 | 18.05910886 | 0.006993007 | BRCA |
| of peptide N- | AOYG_E2- | | | | | | | | |
| term@0 | A150_BH- | | | | | | | | |
| [144.1021]; | A18N_117 | | | | | | | | |
| Oxidation of | C_W_BI_2 | | | | | | | | |
| M@11 | 0130912_ | | | | | | | | |
| [15.9949] | H- | | | | | | | | |
| | PM_f23: | | | | | | | | |
| | 27811: | | | | | | | | |
| | 3 | | | | | | | | |
| iTRAQ 4-plex | TCGA_A2- | 3 | 2403.188171 | 7.887388954 | 2403.207126 | 802.07 | 18.05049453 | 0.005994006 | BRCA |
| of peptide N- | AOT6_E2- | | | | | | | | |
| term@0 | A158_E2- | | | | | | | | |
| [144.1021]; | A15A_117 | | | | | | | | |
| Oxidation of | C_W_BI_2 | | | | | | | | |
| M@1 | 0130918_ | | | | | | | | |
| [15.9949]; | H-292: | | | | | | | | |
| iTRAQ 4- | 3 | | | | | | | | |
| plex of Y@8 | PM_f01: | | | | | | | | |
| [144.1021]; | 28 | | | | | | | | |
| Oxidation of | | | | | | | | | |
| M@11 | | | | | | | | | |
| [15.9949] | | | | | | | | | |
| iTRAQ 4-plex | TCGA_AR | 3 | 2243.108171 | 0.881806441 | 2243.110149 | 8.7741 | 18.04478088 | 0.006993007 | BRCA |
| of peptide N- | A1AP_AN- | | | | | | | | |
| term@0 | AOFK_AO- | | | | | | | | |
| [144.1021]; | AOJ6_117 | | | | | | | | |
| Oxidation of | C_W_BI_2 | | | | | | | | |
| M@1 | 0130517_ | | | | | | | | |
| [15.9949] | H- | | | | | | | | |
| | PM_f22: | | | | | | | | |
| | 34062: | | | | | | | | |
| | 3 | | | | | | | | |
| iTRAQ 4-plex | TCGA_AO | 3 | 2243.108171 | 0.881806441 | 2243.110149 | 748.71 | 18.03674674 | 0.008991009 | BRCA |
| of peptide N- | - | | | | | | | | |
| term@0 | AOJM_C8- | | | | | | | | |
| [144.1021]; | A12V_A8- | | | | | | | | |
| Oxidation of | A08G_117 | | | | | | | | |
| M@1 | C_W_BI_2 | | | | | | | | |
| [15.9949] | 0130927_ | | | | | | | | |
| | H- | | | | | | | | |
| | PM_f23: | | | | | | | | |
| | 30748: | | | | | | | | |
| | 3 | | | | | | | | |
| iTRAQ 4-plex | TCGA_A2- | 3 | 2403.188171 | 7.887388954 | 2403.207126 | 802.07 | 17.97188895 | 0.003996004 | BRCA |
| of peptide N- | AOYG_E2- | | | | | | | | |
| term@0 | A150_BH- | | | | | | | | |
| [144.1021]; | A18N_117 | | | | | | | | |
| Oxidation of | C_W_BI_2 | | | | | | | | |
| M@1 | 0130912_ | | | | | | | | |
| [15.9949]; | H- | | | | | | | | |
| iTRAQ 4- | PM_f03: | | | | | | | | |
| plex of | 25837: | | | | | | | | |
| Y@8 | 3 | | | | | | | | |
| [144.1021]; | | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_ | 3 | 2403.188171 | 7.887388954 | 2403.207126 | 802.07 | 17.87811513 | 0.004995005 | BRCA |
| of peptide N- | AR- | | | | | | | | |
| term@0 | AOTT_AR- | | | | | | | | |
| [144.1021]; | A1AQ_ | | | | | | | | |
| Oxidation | AO- | | | | | | | | |
| of | A12B_117 | | | | | | | | |
| M@1 | C_W_BI_2 | | | | | | | | |
| [15.9949]; | 0131022_ | | | | | | | | |
| iTRAQ 4- | H- | | | | | | | | |
| plex of | PM_f04: | | | | | | | | |
| Y@8 | 28170: | | | | | | | | |
| [144.1021]; | 3 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_ | 3 | 2387.198171 | 5.881508863 | 2387.212211 | 796.74 | 17.10643429 | 0.001998002 | BRCA |
| of peptide N- | AR- | | | | | | | | |
| term@0 | AOTR_AO- | | | | | | | | |
| [144.1021]; | A030_BH- | | | | | | | | |
| Oxidation of | A18R_117 | | | | | | | | |
| M@1 | C_W_BI_2 | | | | | | | | |
| [15.9949]; | 0130825_ | | | | | | | | |
| iTRAQ 4- | H- | | | | | | | | |
| plex of | PM_f24: | | | | | | | | |
| Y@2 | 25366: | | | | | | | | |
| [144.1021] | 3 | | | | | | | | |
| iTRAQ 4-plex | TCGA_AO- | 3 | 2227.118171 | −1.318580439 | 2227.115234 | 743.38 | 16.68997431 | 9.99E−04 | BRCA |
| of peptide N- | A12E_A8- | | | | | | | | |
| term@0 | A06N_A2- | | | | | | | | |
| [144.1021] | AOT1_117 | | | | | | | | |
| | C_W_BI_2 | | | | | | | | |
| | 0130909_ | | | | | | | | |
| | H- | | | | | | | | |
| | PM_f05: | | | | | | | | |
| | 29701: | | | | | | | | |
| | 3 | | | | | | | | |
| iTRAQ 4-plex | TCGA_A2- | 3 | 2227.118171 | −1.318580439 | 2227.115234 | 743.38 | 16.40540448 | 9.99E−04 | BRCA |
| of peptide N- | AOT7_C8- | | | | | | | | |
| term@0 | A12Q_A8- | | | | | | | | |
| [144.1021] | A079_117 | | | | | | | | |
| | C_W_BI_2 | | | | | | | | |
| | 0130820_ | | | | | | | | |
| | H- | | | | | | | | |
| | PM_f05: | | | | | | | | |
| | 30266: | | | | | | | | |
| | 3 | | | | | | | | |
| iTRAQ 4-plex | CPTAC_2 | 3 | 2531.318171 | −1.539583395 | 2531.314273 | 844.78 | 16.3122638 | 0.001998002 | BRCA |
| of peptide N- | 63d3f- | | | | | | | | |
| term@0 | I_blcdb9- | | | | | | | | |
| [144.1021]; | I_c4155b- | | | | | | | | |
| Oxidation of | C_117C_ | | | | | | | | |
| M@1 | W_BI_201 | | | | | | | | |
| [15.9949]; | 40417_H- | | | | | | | | |
| iTRAQ 4- | PM_f01: | | | | | | | | |
| plex of | 20 | | | | | | | | |
| Y@2 | 213: | | | | | | | | |
| [144.1021]; | 3 | | | | | | | | |
| iTRAQ 4- | | | | | | | | | |
| plex of | | | | | | | | | |
| Y@8 | | | | | | | | | |
| [144.1021] | | | | | | | | | |
| iTRAQ 4-plex | TCGA_A2- | 3 | 2387.228171 | −6.685450983 | 2387.212211 | 796.75 | 16.22462066 | 0.002997003 | BRCA |
| of peptide N- | AOSW_AO | | | | | | | | |
| term@0 | -AOJL_BH- | | | | | | | | |
| [144.1021]; | AOBV_117 | | | | | | | | |
| Oxidation of | C_W_BI_2 | | | | | | | | |
| M@1 | 0131024_ | | | | | | | | |
| [15.9949]; | H- | | | | | | | | |
| iTRAQ 4- | PM_f14: | | | | | | | | |
| plex of | 19560: | | | | | | | | |
| Y@2 | 3 | | | | | | | | |
| [144.1021] | | | | | | | | | |
| iTRAQ 4-plex | TCGA_A7- | 4 | 2387.210894 | 0.55163784 | 2387.212211 | 7.8591 | 15.84236377 | 0.004995005 | BRCA |
| of peptide N- | AOCE_BH- | | | | | | | | |
| term@0 | AOCO_A2- | | | | | | | | |
| [144.1021]; | AOYC_117 | | | | | | | | |
| Oxidation of | C_W_BI_2 | | | | | | | | |
| M@1 | 0130524_ | | | | | | | | |
| [15.9949]; | H- | | | | | | | | |
| iTRAQ 4- | PM_f15: | | | | | | | | |
| plex of | 20335: | | | | | | | | |
| Y@2 | 4 | | | | | | | | |
| [144.1021] | | | | | | | | | |
| iTRAQ 4-plex | TCGA_A2- | 3 | 2531.318171 | −1.539583395 | 2531.314273 | 844.78 | 15.38834916 | 9.99E−04 | BRCA |
| of peptide N- | AOEV_AN- | | | | | | | | |
| term@0 | AOAM_D8- | | | | | | | | |
| [144.1021]; | A142_117 | | | | | | | | |
| Oxidation of | C_W_BI_2 | | | | | | | | |
| M@1 | 0130625_ | | | | | | | | |
| [15.9949]; | H- | | | | | | | | |
| iTRAQ 4- | PM_f11: | | | | | | | | |
| plex of | 39 | | | | | | | | |
| Y@2 | 809: | | | | | | | | |
| [144.1021]; | 3 | | | | | | | | |
| iTRAQ 4- | | | | | | | | | |
| plex of | | | | | | | | | |
| Y@8 | | | | | | | | | |
| [144.1021] | | | | | | | | | |
| iTRAQ 4-plex | TCGA_A7- | 3 | 2227.118171 | −1.318580439 | 2227.115234 | 743.38 | 15.22141659 | 0.00999001 | BRCA |
| of peptide N- | AOCJ_AO- | | | | | | | | |
| term@0 | A12F_A2- | | | | | | | | |
| [144.1021] | AOYL_117 | | | | | | | | |
| | C_W_BI_2 | | | | | | | | |
| | 0130805_ | | | | | | | | |
| | H- | | | | | | | | |
| | PM_f15: | | | | | | | | |
| | 35639: | | | | | | | | |
| | 3 | | | | | | | | |
| iTRAQ 4-plex | TCGA_A8- | 3 | 2403.188171 | 7.887388954 | 2403.207126 | 802.07 | 15.0099336 | 0.003996004 | BRCA |
| of peptide N- | A091_C8- | | | | | | | | |
| term@0 | A12L_A2- | | | | | | | | |
| [144.1021]; | AOEX_117 | | | | | | | | |
| Oxidation of | C_W_BI_2 | | | | | | | | |
| M@1 | 0130302_ | | | | | | | | |
| [15.9949]; | H- | | | | | | | | |
| iTRAQ 4- | PM_f04: | | | | | | | | |
| plex of | 29362: | | | | | | | | |
| Y@8 | 3 | | | | | | | | |
| [144.1021]; | | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_A8- | 3 | 2227.118171 | −1.318580439 | 2227.115234 | 743.38 | 14.80312338 | 0.005994006 | BRCA |
| of peptide N- | A091_C8- | | | | | | | | |
| term@0 | A12L_A2- | | | | | | | | |
| [144.1021] | AOEX_117 | | | | | | | | |
| | C_W_BI_2 | | | | | | | | |
| | 0130302_ | | | | | | | | |
| | H- | | | | | | | | |
| | PM_f09: | | | | | | | | |
| | 38904: | | | | | | | | |
| | 3 | | | | | | | | |
| iTRAQ 4-plex | TCGA_A8-1 | 3 | 2403.218171 | −4.595929559 | 2403.207126 | 802.08 | 14.71391547 | 0.008991009 | BRCA |
| of peptide N- | A06Z_A2- | | | | | | | | |
| term@0 | AOD1_A2- | | | | | | | | |
| [144.1021]; | AOCM_11 | | | | | | | | |
| Oxidation of | 7C_W_BI | | | | | | | | |
| M@1 | 20130401_ | | | | | | | | |
| [15.9949]; | H- | | | | | | | | |
| iTRAQ 4- | JQ_f19: | | | | | | | | |
| plex of | 27106: | | | | | | | | |
| Y@2 | 3 | | | | | | | | |
| [144.1021]; | | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_ | 3 | 2227.118171 | −1.318580439 | 2227.115234 | 743.38 | 14.66881737 | 0.002997003 | BRCA |
| of peptide N- | AO- | | | | | | | | |
| term@0 | AOJM_C8- | | | | | | | | |
| [144.1021] | A12V_A8- | | | | | | | | |
| | A08G_117 | | | | | | | | |
| | C_W_BI_2 | | | | | | | | |
| | 0130927_ | | | | | | | | |
| | H- | | | | | | | | |
| | PM_f18: | | | | | | | | |
| | 29383: | | | | | | | | |
| | 3 | | | | | | | | |
| iTRAQ 4-plex | TCGA_A2- | 3 | 2403.218171 | −4.595929559 | 2403.207126 | 802.08 | 14.59007039 | 0.003996004 | BRCA |
| of peptide N- | AOSW_AO | | | | | | | | |
| term@0 | -AOJL_BH- | | | | | | | | |
| [144.1021]; | AOBV_117 | | | | | | | | |
| Oxidation | C_W_BI_2 | | | | | | | | |
| of | 0131024_ | | | | | | | | |
| M@1 | H- | | | | | | | | |
| [15.9949]; | PM_f19: | | | | | | | | |
| İTRAQ 4- | 23582: | | | | | | | | |
| plex of | 3 | | | | | | | | |
| Y@2 | | | | | | | | | |
| [144.1021]; | | | | | | | | | |
| Oxidation | | | | | | | | | |
| of | | | | | | | | | |
| M@11[15.9949] | | | | | | | | | |
| iTRAQ 4-plex | TCGA_A7- | 3 | 2403.218171 | −4.595929559 | 2403.207126 | 802.08 | 14.55807076 | 0.002997003 | BRCA |
| of peptide N- | AOCD_C8- | | | | | | | | |
| term@0 | A12W_ | | | | | | | | |
| [144.1021]; | AN- | | | | | | | | |
| Oxidation of | AOAL_117 | | | | | | | | |
| M@1 | C_W_BI_2 | | | | | | | | |
| [15.9949]; | 0130913_ | | | | | | | | |
| iTRAQ 4- | H- | | | | | | | | |
| plex of | PM_f19: | | | | | | | | |
| Y@2 | 30242: | | | | | | | | |
| [144.1021]; | 3 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_A7- | 3 | 2371.208171 | 3.848567666 | 2371.217296 | 791.41 | 14.38165333 | 0.008991009 | BRCA |
| of peptide N- | AOCD_C8- | | | | | | | | |
| term@0 | A12W_ | | | | | | | | |
| [144.1021]; | AN- | | | | | | | | |
| iTRAQ | AOAL_117 | | | | | | | | |
| 4-plex of | C_W_BI_2 | | | | | | | | |
| Y@8 | 0130913_ | | | | | | | | |
| [144.1021] | H- | | | | | | | | |
| | PM_f03: | | | | | | | | |
| | 10929: | | | | | | | | |
| | 3 | | | | | | | | |
| iTRAQ 4-plex | TCGA_A8- | 3 | 2227.118171 | −1.318580439 | 2227.115234 | 743.38 | 14.34757509 | 0.008991009 | BRCA |
| of peptide N- | A091_C8- | | | | | | | | |
| term@0 | A12L_A2- | | | | | | | | |
| [144.1021] | AOEX_117 | | | | | | | | |
| | C_W_BI_ | | | | | | | | |
| | 20130302_ | | | | | | | | |
| | H- | | | | | | | | |
| | PM_f19: | | | | | | | | |
| | 26206: | | | | | | | | |
| | 3 | | | | | | | | |
| ITRAQ 4-plex | CPTAC_2 | 3 | 2403.188171 | 7.887388954 | 2403.207126 | 802.07 | 14.11660881 | 0.003996004 | BRCA |
| of peptide N- | 63d3f- | | | | | | | | |
| term@0 | blcdb9- | | | | | | | | |
| [144.1021]; | I_c4155b- | | | | | | | | |
| Oxidation of | C_117C_ | | | | | | | | |
| M@1 | W_BI_ | | | | | | | | |
| [15.9949]; | 201398: | | | | | | | | |
| iTRAQ 4- | 340417_ | | | | | | | | |
| plex of | H- | | | | | | | | |
| Y@2 | PM_f11: | | | | | | | | |
| [144.1021]; | 24 | | | | | | | | |
| Oxidation | | | | | | | | | |
| of | | | | | | | | | |
| M@11 | | | | | | | | | |
| [15.9949] | | | | | | | | | |
| iTRAQ 4-plex | TCGA_AO | 3 | 2243.108171 | 0.881806441 | 2243.110149 | 748.71 | 14.04648282 | 0.006993007 | BRCA |
| of peptide N- | -AOJC_A8- | | | | | | | | |
| term@0 | A08Z_AR- | | | | | | | | |
| [144.1021]; | AOTX_117 | | | | | | | | |
| Oxidation | C_W_BI_2 | | | | | | | | |
| of | 0130527_ | | | | | | | | |
| M@1 | H- | | | | | | | | |
| [15.9949] | PM_f23: | | | | | | | | |
| | 30839: | | | | | | | | |
| | 3 | | | | | | | | |
| iTRAQ 4-plex | TCGA_A2- | 3 | 2531.318171 | −1.539583395 | 2531.314273 | 844.78 | 12.96072217 | 0.004995005 | BRCA |
| of peptide N- | AOYM_BH | | | | | | | | |
| term@0 | - | | | | | | | | |
| [144.1021]; | AOC7_A2- | | | | | | | | |
| Oxidation | AOSX_117 | | | | | | | | |
| of | C_W_BI_2 | | | | | | | | |
| M@1 | 0131025_ | | | | | | | | |
| [15.9949]; | H- | | | | | | | | |
| İTRAQ 4- | PM_f24: | | | | | | | | |
| plex of | 33487: | | | | | | | | |
| Y@2 | 3 | | | | | | | | |
| [144.1021]; | | | | | | | | | |
| iTRAQ 4- | | | | | | | | | |
| plex of | | | | | | | | | |
| Y@8 | | | | | | | | | |
| [144.1021] | | | | | | | | | |
| iTRAQ 4-plex | TCGA_D8 | 3 | 2531.318171 | −1.539583395 | 2531.314273 | 844.78 | 12.94380806 | 0.003996004 | BRCA |
| of peptide N- | -A13Y_A8- | | | | | | | | |
| term@0[144. | A076_AO- | | | | | | | | |
| 1021]; | A126_117 | | | | | | | | |
| Oxidation of | C_W_BI_2 | | | | | | | | |
| M@1[15.994 | 0130617_ | | | | | | | | |
| 9]; | H- | | | | | | | | |
| İTRAQ 4- | PM_f23: | | | | | | | | |
| plex of | 43263: | | | | | | | | |
| Y@2 | 3 | | | | | | | | |
| [144.1021]; | | | | | | | | | |
| iTRAQ 4- | | | | | | | | | |
| plex of | | | | | | | | | |
| Y@8 | | | | | | | | | |
| [144.1021] | | | | | | | | | |

Peptide: SGSLQGTTEPSMTHSIIASTSL (SEQ ID NO: 21)

| | | | | | | | | | |
|---|---|---|---|---|---|---|---|---|---|
| TMT 10-plex | 12CPTAC_ | 3 | 2449.233542 | −5.151682333 | 2449.220924 | 817.418457 | 13.47138227 | 0.004995005 | LUAD |
| of peptide N- | LUAD_W_ | | | | | | | | |
| term@0 | BI_20180 | | | | | | | | |
| [229.1629]; | 912_KL_ | | | | | | | | |
| Oxidation of | f08: | | | | | | | | |
| M@12 | 34207: | | | | | | | | |
| [15.9949] | 3 | | | | | | | | |
| iTRAQ 4-plex | TCGA_AO | 5 | 2348.163618 | 0.648202166 | 2348.16514 | 470.64 | 17.11418468 | 0.00999001 | BRCA |
| of peptide N- | - | | | | | | | | |
| term@0 | A12D_C8- | | | | | | | | |
| [144.1021] | A131_AO- | | | | | | | | |
| | A12B_117 | | | | | | | | |
| | C_W_BI_2 | | | | | | | | |
| | 0130208_ | | | | | | | | |
| | H- | | | | | | | | |
| | PM_f04: | | | | | | | | |
| | 5856: | | | | | | | | |
| | 5 | | | | | | | | |
| iTRAQ 4-plex | TCGA_D8 | 3 | 2364.158171 | 0.796803779 | 2364.160054 | 789.06 | 16.06480776 | 0.001998002 | BRCA |
| of peptide N- | -A13Y_A8- | | | | | | | | |
| term@0 | A076_AO- | | | | | | | | |
| [144.1021]; | A126_117 | | | | | | | | |
| Oxidation of | C_W_BI_2 | | | | | | | | |
| M@12 | 0130617_ | | | | | | | | |
| [15.9949] | H- | | | | | | | | |
| | PM_f14: | | | | | | | | |
| | 17026: | | | | | | | | |
| | 3 | | | | | | | | |
| iTRAQ 4-plex | TCGA | 4 | 2364.170894 | −4.585037084 | 2364.160054 | 592.05 | 15.91237957 | 0.002997003 | BRCA |
| of peptide N- | _BH- | | | | | | | | |
| term@0 | AOEE_AO- | | | | | | | | |
| [144.1021]; | AOJ9_BH- | | | | | | | | |
| Oxidation of | AOE0_117 | | | | | | | | |
| M@12 | C_W_BI_2 | | | | | | | | |
| [15.9949] | 0130412_ | | | | | | | | |
| | H- | | | | | | | | |
| | PM_f16: | | | | | | | | |
| | 19019: | | | | | | | | |
| | 4 | | | | | | | | |
| iTRAQ 4-plex | TCGA_ | 4 | 2364.170894 | −4.585037084 | 2364.160054 | 592.05 | 13.74961469 | 9.99E−04 | BRCA |
| of peptide N- | AN- | | | | | | | | |
| term@0 | AOFL_BH- | | | | | | | | |
| [144.1021]; | AODG_ | | | | | | | | |
| Oxidation of | AN- | | | | | | | | |
| M@12 | AOAS_117 | | | | | | | | |
| [15.9949] | C_W_BI_2 | | | | | | | | |
| | 0130726_ | | | | | | | | |
| | H- | | | | | | | | |
| | PM_f02: | | | | | | | | |
| | 5274: | | | | | | | | |
| | 4 | | | | | | | | |
| iTRAQ 4-plex | TCGA_A2- | 4 | 2364.170894 | −4.585037084 | 2364.160054 | 592.05 | 13.49307705 | 0.001998002 | BRCA |
| of peptide N- | AOT7_C8- | | | | | | | | |
| term@0 | A12Q_A8- | | | | | | | | |
| [144.1021]; | A079_117 | | | | | | | | |
| Oxidation of | C_W_BI_2 | | | | | | | | |
| M@12 | 0130820_ | | | | | | | | |
| [15.9949] | H- | | | | | | | | |
| | PM_f18: | | | | | | | | |
| | 17728: | | | | | | | | |
| | 4 | | | | | | | | |
| iTRAQ 4-plex | TCGA_ | 3 | 2348.168171 | −1.290730299 | 2348.16514 | 783.73 | 12.23769746 | 0.00999001 | BRCA |
| of peptide N- | C8- | | | | | | | | |
| term@0 | A12P_BH- | | | | | | | | |
| [144.1021] | AOC1_A2- | | | | | | | | |
| | AOEY_117 | | | | | | | | |
| | C_W_BI_2 | | | | | | | | |
| | 0130622_ | | | | | | | | |
| | H- | | | | | | | | |
| | PM_f04: | | | | | | | | |
| | 35009: | | | | | | | | |
| | 3 | | | | | | | | |

In addition, ribosome profiling (Ribo-seq) analysis from public datasets shows a focal enrichment in the predicted ORF within exon 4 of the BCAR4 transcript (see e.g., FIG. 13C). These data are strong evidence that BCAR4 encodes an expressed protein in solid tumors.

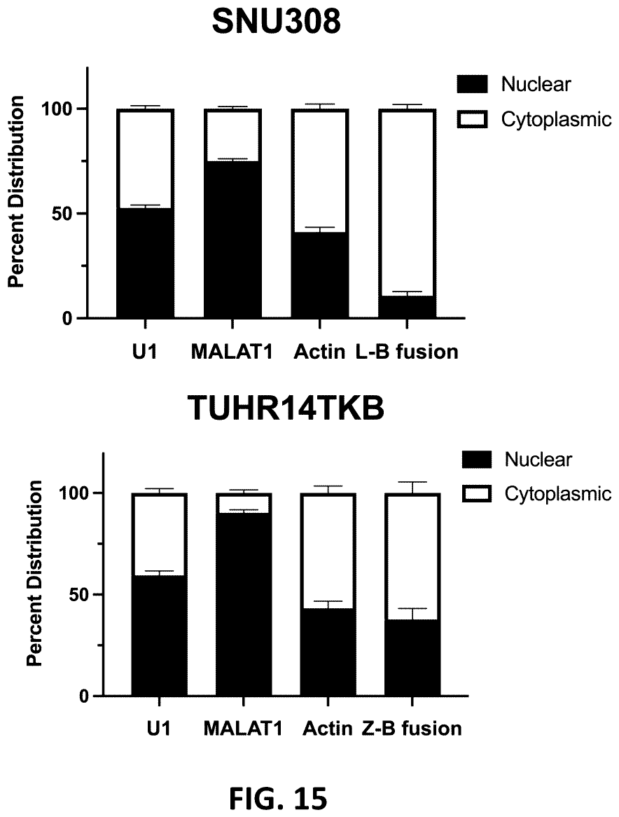

The BCAR4 protein contributes to cancer phenotypes; however, there is also evidence that BCAR4 functions as a lncRNA. For insight into the function of BCAR4, the localization of BCAR4 fusion transcripts was assayed: cytoplasmic localization would indicate translation potential while nuclear localization would indicate a noncoding function of BCAR4. Endogenously expressed BCAR4 fusion transcripts localized predominantly to the cytoplasm: 89% of LITAF-BCAR4 RNA in SNU308 cells and 62% of ZC3H7A-BCAR4 RNA in TUHR14TKB cells (see e.g., FIG. 15). These data are consistent with translation of the BCAR4 ORF.

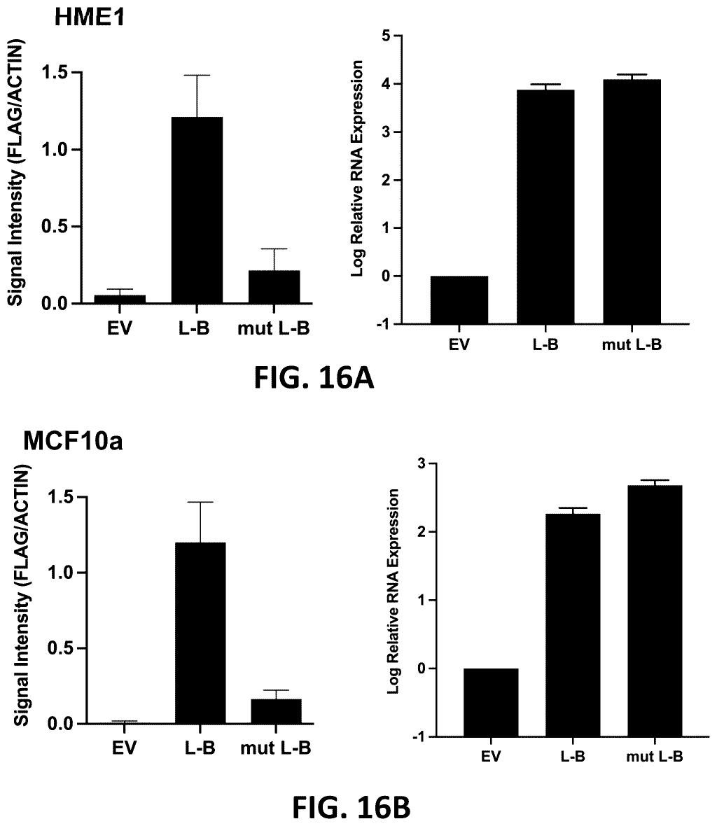

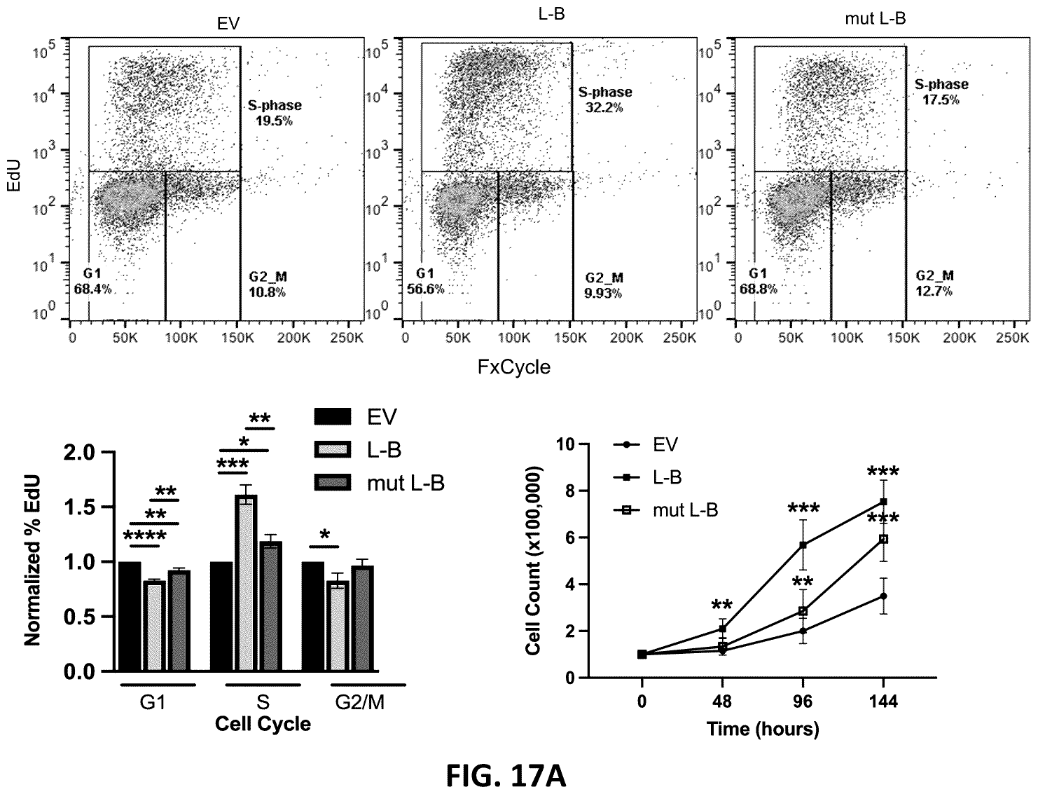

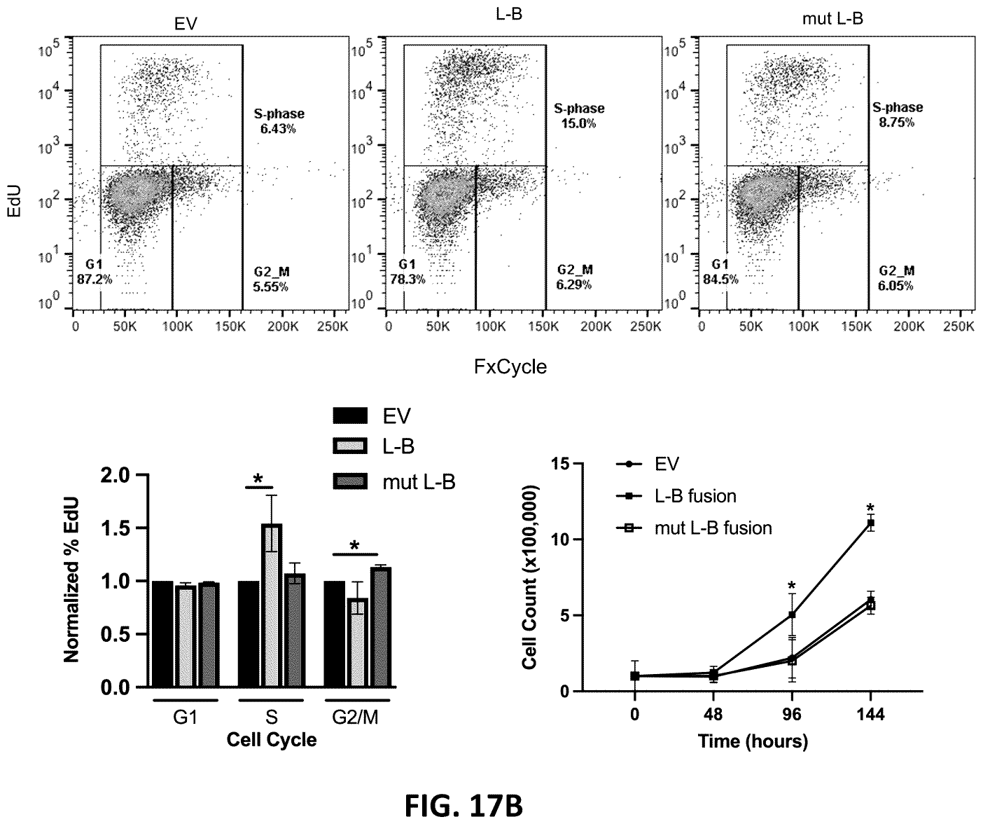

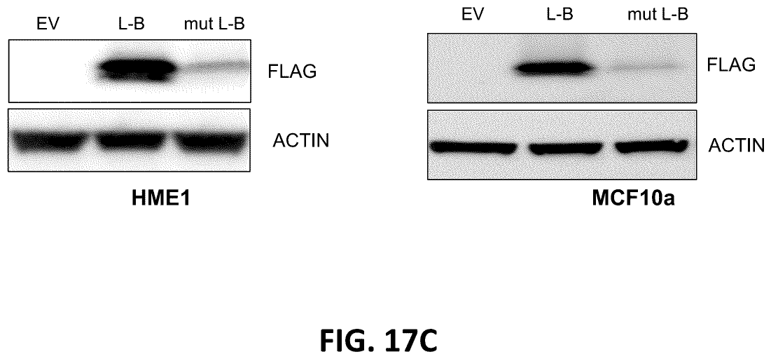

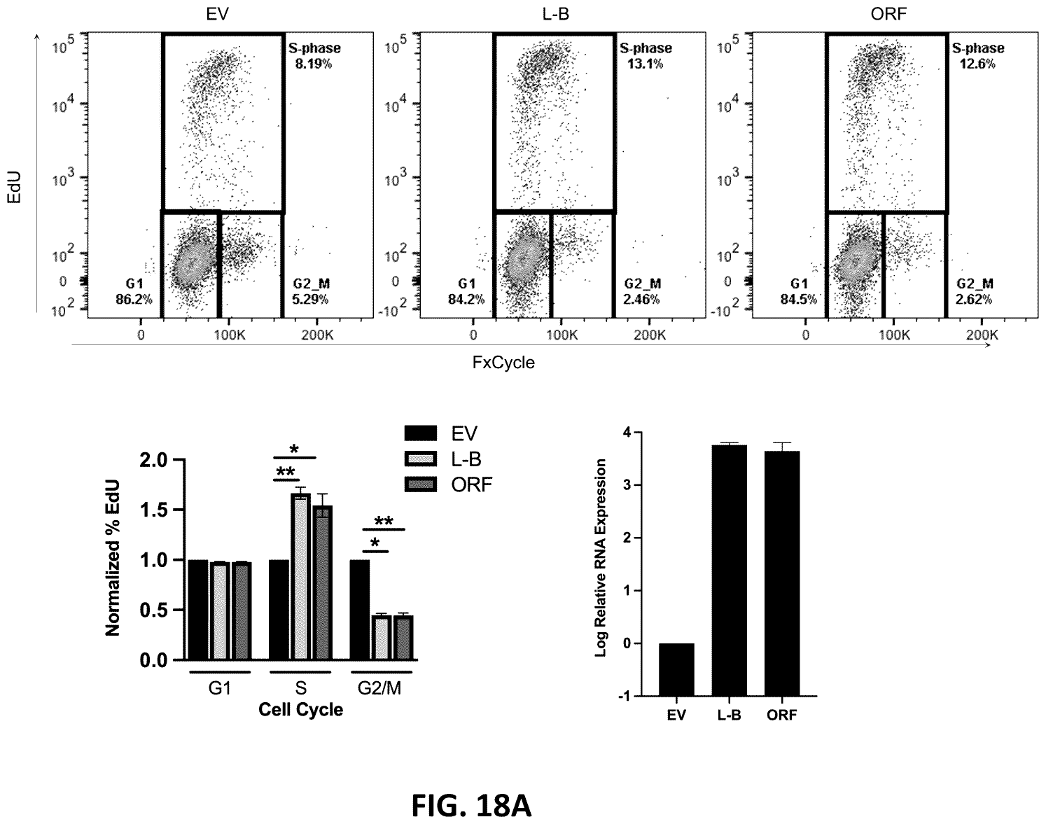

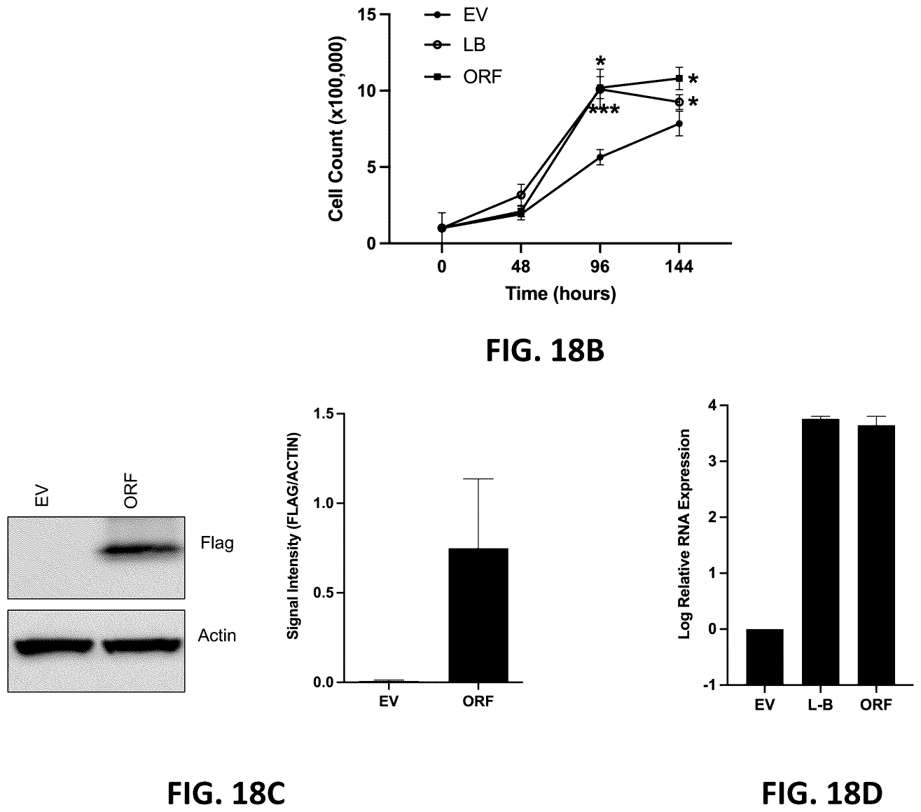

To directly test the functional significance of the BCAR4 ORF, the start codon in the LITAF-BCAR4 fusion plasmid was mutated, thus abrogating potential translation of the BCAR4 ORF (see e.g., FIG. 6). HME1 and MCF10a cells expressing the mutant L-B fusion had significantly fewer S-phase cells than wild-type L-B fusion expressing cells, indicating that translation of the ORF is at least partly responsible for the cell-cycle phenotype (see e.g., FIG. 17A and FIG. 171B). Over time these differences in cell-cycle progression led to large changes in cell number: 96 hours after plating, there were twice as many HME1 cells expressing the L-B fusion than the mutant L-B fusion (see e.g., FIG. 17A). Changes in cell-cycle and cell number were also observed in MCF10a cells (see e.g., FIG. 17B). To directly detect BCAR4 protein in the models, the constructs were FLAG-tagged and robust expression was observed in cells expressing the L-B fusion but not the mutant L-B fusion (see e.g., FIG. 17C and FIG. 16A-FIG. 16B). Some residual signal was observed from the mutant L-B construct, potentially caused by use of a second in-frame ATG codon just downstream of the canonical start codon. Expression of the BCAR4 ORF alone (without additional noncoding sequence) similarly promoted cell-cycle progression and proliferation in MCF10a cell models (see e.g., FIG. 18A-FIG. 18D). Overall, these data show that the fourth exon of BCAR4 can produce a small peptide capable of inducing cell growth.

Discussion

Using a highly sensitive gene fusion discovery tool, INTEGRATE, a novel gene fusion activating a known oncogene, BCAR4, was discovered. This is the first unbiased pan-cancer study to systematically discover functionally recurrent BCAR4 gene fusions across cancer types. These findings have clinical significance as (i) full-length BCAR4 is implicated in poor clinical outcome and treatment resistance; (ii) full-length BCAR4 has oncogenic potential in breast cancer and NSCLC; and (iii) focal amplifications of BCAR4 were found in 20% of cervical patients in the TCGA dataset. The findings extend the clinical significance of BCAR4 into new cancer types-including highly aggressive cancers- to provide an important mechanism of how BCAR4 induces oncogenic properties.

The findings indicate that BCAR4 chimeras are oncogenic driver events across cancer types. 47 BCAR4 gene fusions were discovered with 19 different 5′ partners-mostly involving only the first untranslated regulatory exon of the 5′ gene. A previous study in NCSLC also found BCAR4 gene fusions with the 5′ partner CD63. This pan-cancer study expands beyond lung cancer to discover BCAR4 gene fusion events in 11 cancers, which surpasses the prevalence of well characterized functionally recurrent gene fusions such as EVT and ROS (two cancers), ERG (four cancers), BRAF and ALK (six cancers). The consistent expression of exon 4 of BCAR4 suggests functional recurrence and implicates a biological and clinical importance of BCAR4 gene fusions in solid tumors.

It is challenging to confidently annotate RNAs as coding or noncoding with growing evidence suggesting RNAs may encode small ORFs producing stable, functional peptides. Complicating this process is the possibility that RNAs can have both coding and noncoding functions. Full length BCAR4 was first classified as a protein with expression in human oocytes and placenta; however, more recently is it described as a lncRNA, raising the possibility that BCAR4 may function as a noncoding RNA and/or a protein-coding gene. Herein is shown that the minimum common region of BCAR4 across all gene fusions is the fourth exon, which harbors an ORF. The computational and experimental data provide evidence for translation of the ORF to produce a functional protein capable of influencing oncogenic phenotypes. This is supported by detecting BCAR4 peptide in patient tumors and FLAG-tagged BCAR4 protein in cell culture models. Fusion and ORF-only constructs had significantly increased proliferation in benign cell lines while a construct with a mutated ATG start site had significantly diminished protein expression and proliferation. Genomic editing could provide additional evidence for the importance of the peptide relative to the lncRNA function. These results establish BCAR4 fusions and their resulting peptide products as potential oncogenic drivers and possible therapeutic targets.

Materials and Methods

TCGA Datasets

The aligned BAM files at for the cancer types included in the TCGA Pan-Cancer Analysis listed in TABLE 1 were downloaded.

TABLE 1
Summary of TCGA cohorts and sample sizes.

| | | | Matched |
|---|---|---|---|
| | | | Normals |
| | | Tumors with | with |
| Cohort | Cancer Type | RNA-Seq | RNA-Seq |
| ACC | Adrenocortical carcinoma | 79 | 0 |
| BLCA | Bladder Urothelial Carcinoma | 408 | 19 |
| BRCA | Breast Invasive Carcinoma | 1092 | 113 |
| CESC | Cervical squamous cell | 303 | 3 |
| | carcinoma and endocervical | | |
| | adenocarcinoma | | |
| CHOL | Cholangiocarcinoma | 36 | 9 |
| CRC | Colon Adenocarcinoma (COAD) | 387 | 51 |
| | and Rectum Adenocarcinoma | | |
| | (READ) | | |
| DLBC | Lymphoid Neoplasm Diffuse | 48 | 0 |
| | Large B-cell Lymphoma | | |
| ESCA | Esophageal carcinoma | 164 | 10 |
| GBM | Glioblastoma multiforme | 166 | 5 |
| HNSC | Head and Neck squamous cell | 501 | 44 |
| | carcinoma | | |
| KIRH | Kidney Chromophobe | 66 | 24 |
| KIRC | Kidney Renal Cell Carcinoma | 530 | 72 |
| KIRP | Kidney renal papillary cell | 289 | 32 |
| | carcinoma | | |
| LAML | Acute Myeloid Leukemia | 151 | 0 |
| LGG | Brain Lower Grade Glioma | 512 | 0 |
| LIHC | Liver hepatocellular carcinoma | 371 | 50 |
| LUAD | Lung Adenocarcinoma | 515 | 59 |
| LUSC | Lung squamous cell carcinoma | 501 | 49 |
| MESO | Mesothelioma | 86 | 0 |
| OV | Ovarian serous | 376 | 0 |
| | cystadenocarcinoma | | |
| PAAD | Pancreatic adenocarcinoma | 177 | 4 |
| PCPG | Pheochromocytoma and | 179 | 3 |
| | Paraganglioma | | |
| PRAD | Prostate adenocarcinoma | 496 | 52 |
| SARC | Sarcoma | 259 | 2 |
| SKCM | Skin Cutaneous Melanoma | 468 | 1 |
| STAD | Stomach adenocarcinoma | 378 | 32 |
| TGCT | Testicular Germ Cell Tumors | 150 | 0 |
| THCA | Thyroid carcinoma | 502 | 58 |
| THYM | Thymoma | 119 | 2 |
| UCEC | Uterine Corpus Endometrial | 194 | 24 |
| | Carcinoma | | |
| UCS | Uterine Carcinosarcoma | 56 | 0 |
| UVM | Uveal Melanoma | 79 | 0 |
| Total | | 9638 | 349 |

When available, RNA sequencing (RNA-seq) BAM files for matched adjacent normal tissue were also downloaded. Following TCGA practices, COAD and READ were merged to form a colorectal cancer cohort.

Gene Fusion Discovery

INTEGRATE is an open-source gene fusion discovery tool designed to map gene fusions using aligned RNA-seq reads and whole genome sequencing (WGS) paired-end sequencing reads, if available. INTEGRATE version 0.2.6 was run using default parameters in “RNA only” mode on the aligned RNA-seq reads. Analysis was based on hg38. Functionally recurrent 3′ fusion partners were identified if a gene was only found as a 3′ partner in somatic gene fusions across patients. Summary figures of novel and previously reported functionally recurrent 3′ fusion partners were generated using R (version 4.0.5).

Cell Culture

SNU308 and TUHR14TKB were selected for their endogenous expression of BCAR4 gene fusions with no expression of full length BCAR4. The TUHR14TKB cell line was purchased from RIKEN BRC Cell Engineering Division (RCB1383; RRID_CVCL_5953). SNU308 gallbladder cell line was purchased from the Korean Cell Line Bank (RRID:CVCL_5048). Cell lines were grown in RPMI-1640 (Genesee Scientific) supplemented with 10% FBS (Sigma) and 1% penicillin/streptomycin (Thermo Fisher Scientific). hTER1-HME1 were purchased from ATCC (AlTCC, catalog no, CRL-10317, RRID: CVCL_0598). M:F10a were from Dr. Ron Bose (Washington University; St. Louis, MO). Cell lines were cultured in MEGM Mammary Epithelial Cell Growth Medium BulletKit (Lonza) or following the Brugge Lab protocol. Cell line authentication and validation were performed by ATCC, RIKEN, and Korean Cell Line Bank. Cells were passaged less than 10 times and monitored for Mycoplasma by PCR.

Transfections and Overexpression Models

Cells were transfected with 10 nmol/L of custom silencer select siRNA (see e.g., TABLE 2) and Lipofectamine RNAiMax (Thermo Fisher Scientific) following the manufacturer's protocol and used for assays 72 hours later.

TABLE 2
siRNA and primer sequence

| SIRNA | | |
|---|---|---|
| name | 5′3′ Forward | 5′3′ Reverse |
| Fusion | GACUAGGAGUGAUACGA | UUUCGUAUCACUCCUAG |
| SIRNA 1 | AA (SEQ ID NO: 4) | UC (SEQ ID NO: 5) |
| Fusion | CGCUGUAGUUGUACAUU | UCAAUGUACAACUACAG |
| SIRNA 2 | GA (SEQ ID NO: 6) | CC (SEQ ID NO: 7) |
| Primers | | |
| Gene | Forward Primer | Reverse Primer |
| RPL32 | AGGCATTGACAACAGGG | GTTGCACATCAGCAGCA |
| | TTC (SEQ ID NO: 8) | CTT (SEQ ID NO: 9) |
| LITAF- | CGAGAGGCCAGCTCAG | TCAGAGCAAGACAAGCA |
| BCAR4 | AC (SEQ ID NO: 10) | TCG (SEQ ID NO: 11) |
| ZC3HTA- | GCTAACCGAGGGAGAGC | GGAACTCCCGTCTTCTGG |
| BCAR4 | TG (SEQ ID NO: 12) | AT (SEQ ID NO: 13) |
| U1 | GGGAGATACCATGATCAC | CCACAAATTATGCAGTCG |
| | GAAGGT | AGTTTCCC |
| | (SEQ ID NO: 14) | (SEQ ID NO: 15) |
| MALAT1 | GACGGAGGTTGAGATGA | ATTCGGGGCTCTGTAGTC |
| | AGC (SEQ ID NO: 16) | CT (SEQ ID NO: 17) |
| ACTIN | CTCGACACCAGGGCGTT | CCACTCCATGCTCGATAG |
| | ATG (SEQ ID NO: 18) | GAT (SEQ ID NO: 19) |

Fusion constructs were synthesized by Invitrogen and Infusion cloned (Takara) into the pCFG5-IEGZ plasmid (from Dr. Ron Bose). A terminal FLAG-tag was added to the BCAR4 ORF and Infusion cloned. Sequences were confirmed by Sanger sequencing at Genewiz (South Plainfield). 293T cells were transfected with 3.75 mg of expression plasmid and an 8:1 ratio of pUMVC: VSVG. The next day media was exchanged for cell-specific complete media and virus collected at 48 and 72 hours. 2 mL of virus with 8 mg/mL Polybrene (Sigma) was added to mammalian cells, centrifuged at 2,500 RPM for 75 minutes, and fresh media exchanged after 6 hours. Cells were incubated with virus/polybrene for 6 hours the next day and used for assays 48 hours later. Point mutations within the ATG start site were introduced with Q5 site-directed mutagenesis Kit (NEB) to create the LITAF-BCAR4 L-B mutant construct.

RNA Isolation and cDNA Synthesis

Total RNA was isolated with NucleoSpin RNA plus with DNA removal column (Macherey-Nagel). cDNA was synthesized with High Capacity cDNA Reverse Transcription Kit (Invitrogen) or 1-Step TB Green PrimeScript qRT-PCR kit (Takara).

qRT-PCR

Gene expression was confirmed with qRT-PCR using PowerSyBr Green (Invitrogen) or 1-Step TB Green PrimeScript (Takara). The comparative CT (DDCT) method was used with values normalized to the housekeeping gene, RPL32, and to control samples. All primers (see e.g., TABLE 2) were obtained from Integrated DNA Technologies and determined to have 90% to 110% primer efficiency.

FITC Annexin V Apoptosis Detection

Cells were seeded at 200,000 cells/well and Annexin V staining determined 2 days later according to the manufacturer's protocol (BD Pharmingen).

Cellular Nuclear and Cytoplasmic Isolation

Cells were isolated according to the PARIS kit protocol (Thermo Fisher Scientific) and gene expression determined by qPCR. Nuclear and cytoplasmic isolations were calculated by normalizing respective genes to total RNA expression.

Cell Counting

Cells were seeded at 100,000 cells/well and counted every 2 days using the Countess II FL Automated Cell Counter (Thermo Fisher Scientific).

EdU Proliferation Assay

Cells were seeded at 250,000 cells/well and treated 2 days later with 10 mmol/L EdU (Thermo Fisher Scientific) for 2 hours. Cells were processed following the manufacturer's instructions and stained for DNA content with FxCycle Violet (Thermo Fisher Scientific). Analysis was performed on a FACScan flow cytometer (Becton Dickinson) at the Siteman Cancer Center Flow Cytometry Core (St. Louis, MO). A minimum of 10,000 events per sample were collected. FlowJo Version10 (RRID:SCR_008520; Becton Dickinson) was used to analyze data.

Western Blotting

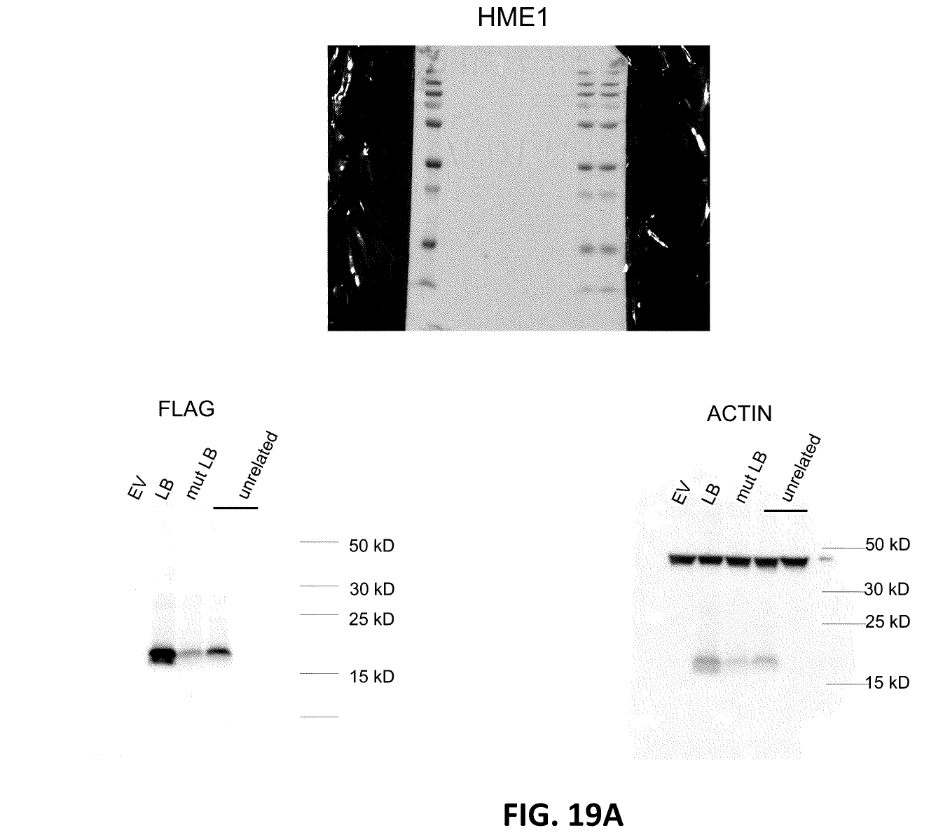

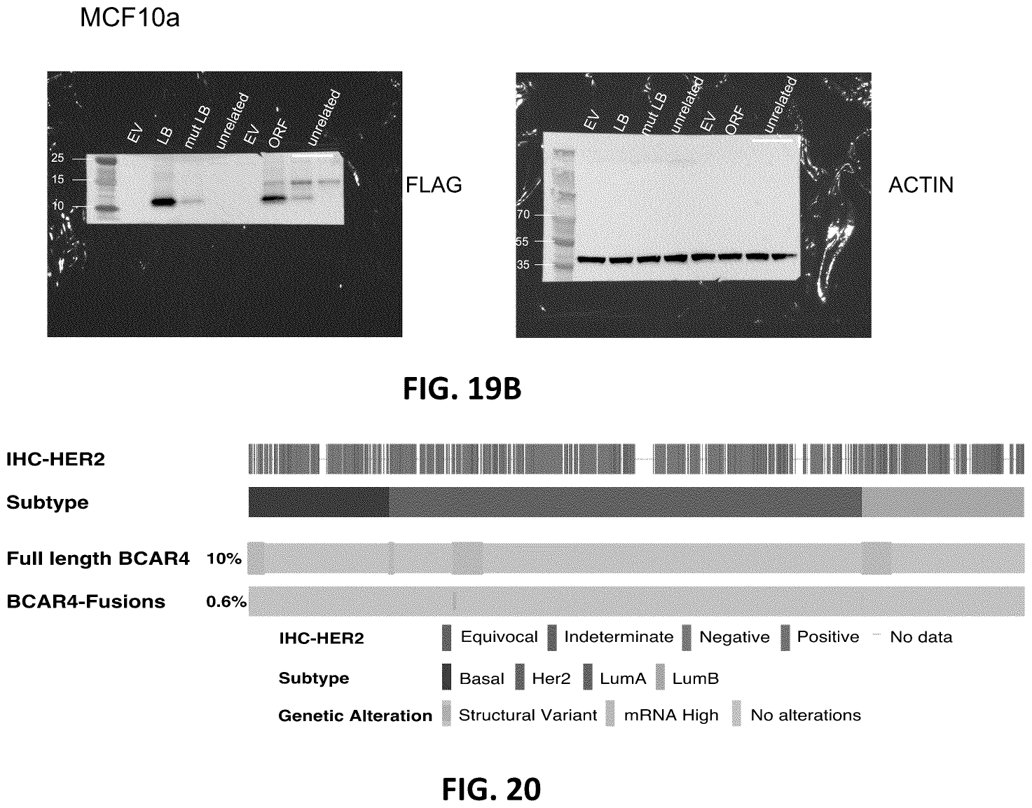

Antibodies against FLAG (2368S; RRID:AB_2217020, 1:1000; Cell Signaling Technology), actin (3700S; RRID:AB_2242334, 1:10,000; Cell Signaling Technology), Goat anti-mouse or anti-rabbit peroxidase-conjugated secondary antibodies (Thermo Fisher Scientific) were used for experiments. Protein samples were prepared with ice-cold RIPA lysis buffer (25 mmol/L Tris pH7.5, 1% NP40, 0.1% SDS, 150 mmol/L NaCl, 0.5% sodium deoxycholate, and 1× Halt Protease Inhibitor Cocktail; Thermo Fisher Scientific). Lysate was subjected to a short sonication and then clarified by centrifugation at maximum speed, 4° C. for 10 minutes. Protein concentration was determined with the DC Protein Assay (BioRad). Samples were diluted in loading buffer, boiled, and equal protein concentrations (20-30 mg) loaded and resolved by SDS-polyacrylamide gel electrophoresis in 12% or 4% to 12% Bolt Bis-Tris precast gels (Invitrogen). Gels were transferred at 60° C. for 1 hour to a nitrocellulose membrane (BioRad). Proteins were detected with specific antibodies and visualized and quantified on the ChemDoc MP Imaging System (BioRad) using secondary antibodies and Clarity Western ECL Substrate (Thermo Fisher Scientific). Uncropped western blots are shown in FIG. 19A-FIG. 19B.

Small Peptide Prediction and Validation

For the proteogenomic search, ORFs predicted in transcripts of lncRNAs annotated in LNCipedia appended to canonical proteins from Uniprot (RRID:SCR_002380) were used to ensure that the proteogenomic database is not biased towards the discovery of the BCAR4 ORF. An equal number of reversed decoys was used to utilize the target-decoy strategy setting with a threshold of 0.01 for the FDR. Raw mass spectrometry files in mzML format were downloaded from the Clinical Proteomic Tumor Analysis Consortium data resource (CPTAC; RRID:SCR_017135). Sequences between start codons (AUG, CUG, UUG) and stop codons (UAG, UGA, UAA) in each of the forward translated frames, with a minimum length of 100 nucleotides, were used as putative ORFs in lncRNAs for the construction of the proteogenomic database. This minimum threshold was selected to maximize signal to noise ratio: encompassing the discovery of known small ORFs (HOXB-AS3/LOC100507537) without increasing false positives from shorter peptides. TABLE 3 shows the variable and fixed modifications used for each protein labeling protocol.

TABLE 3
Proteomic variable and fixed modifications to label protein.

| Protocol | Modification(s) |
|---|---|
| TMT | fixed Cystine carbamidomethylation (+57.0215 |
| | fixed Lysine TMT labeling (+229.1629) |
| | variable Methionine oxidation (+15.9949) |
| | variable N-terminal protein acetylation (+42.0106) |
| | variable TMT labeling of peptide N terminus and serine residues |
| iTRAQ | fixed Cysteine carbamidomethylation (+57.0215) |
| | fixed iTRAQ labeling of lysine (+144.10253) |
| | fixed peptide N terminus |
| | variable Methionine oxidation (+15.9949) |

The MSFragger search engine was used allowing semitryptic peptides, two missed cleavage sites, 12C/13C isotope errors, and a precursor-ion mass tolerance of 20 ppm. PeptideProphet (RRID:SCR_000274) and ProteinProphet (RRID:SCR_000286) were used to process the search engine results and infer protein groups, respectively. To further validate the novel peptides from the proteogenomic search, the global FDR filtering step was followed by peptide-centric validation as implemented in PepQuery and it was verified that the identified peptides from the BCAR4 ORF are indeed statistically significant (PepQuery P≤0.01). PSIPRED Protein Analysis Workbench (RRID:SCR_010246) was used to predict protein secondary structure and InterProScan (RRID:SCR_005829) to identify protein domains.

### Example 2: BCAR4 as a Predictive Biomarker and Therapeutic Target Across Cancers

This example describes how the BCAR4 gene represents a novel indicator of HER2 non-amplified patients that also have poor outcome and could predict treatment response.

Breast cancer is the most common cancer diagnosis in women worldwide. Disease heterogeneity necessitates continued and improved molecular profiling to improve patient care. Described herein is a study investigating how the BCAR4 gene represents a novel indicator of HER2 non-amplified patients that also have poor outcome and may predict treatment response. The long-term goal is to monitor BCAR4 as a (i) prognostic marker to identify patients that need more aggressive treatment options and subsequently as a (ii) predictive biomarker to select appropriate treatment strategies. The data suggest BCAR4 is clinically relevant to identifying patients previously excluded from—but could respond to—multiple FDA-approved HER2-targeted therapies. This has immediate and broad clinical impact since BCAR4 is altered across multiple solid tumors thereby expanding the use of existing HER2-therapies beyond breast cancer.

BCAR4 can be Activated by Somatic Mutations Across Solid Tumors

Gene fusions are specific diagnostic markers, prognostic indicators, and therapeutic targets in cancer. The activation of Breast Cancer Anti-Estrogen Resistant 4 (BCAR4) represents the 3^{rd} most prevalent 5′ parent in the pan cancer analysis (see e.g., Example 1). Activation of the 4^{th} exon of BCAR4 is more prevalent than other clinically actionable gene fusion discoveries.

BCAR4 as a Prognostic Biomarker

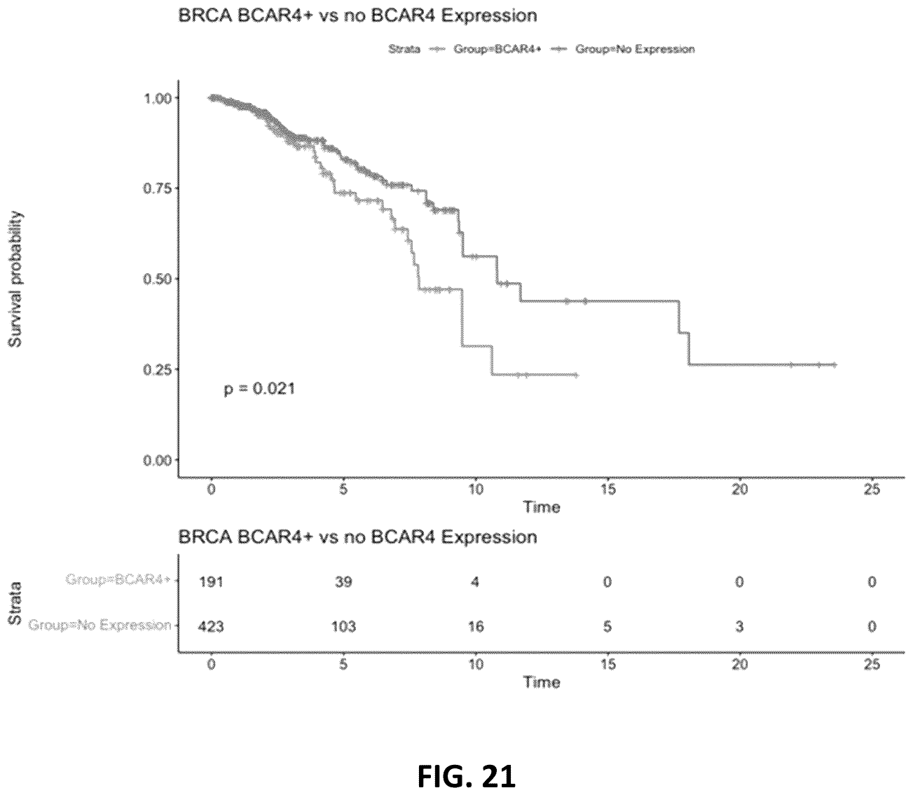

Approximately 10% of breast cancer patients in The Cancer Genome Atlas cohort (TCGA) have BCAR4 overexpression (gene fusion events and full-length expression). Notably, these patients are predominately HER2-negative (nonamplified) in the Luminal A or B subtype classification (see e.g., FIG. 20). When patients are stratified on BCAR4 expression the overall outcome for patient overall survival (OS) is significantly worse for the BCAR4-expressing patients (red line; see e.g., FIG. 21)

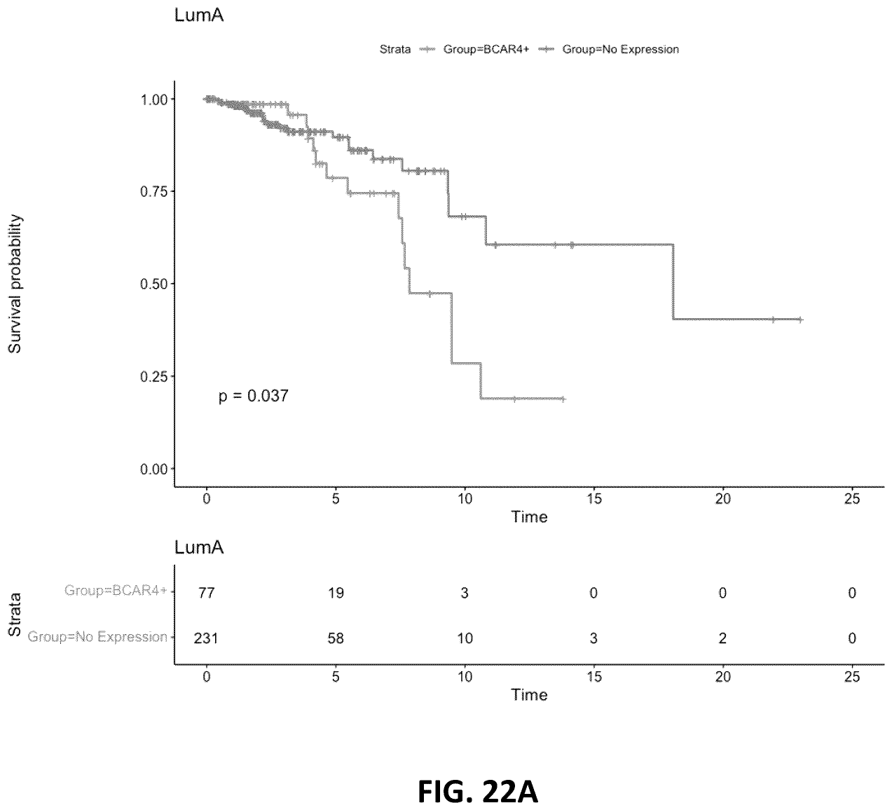

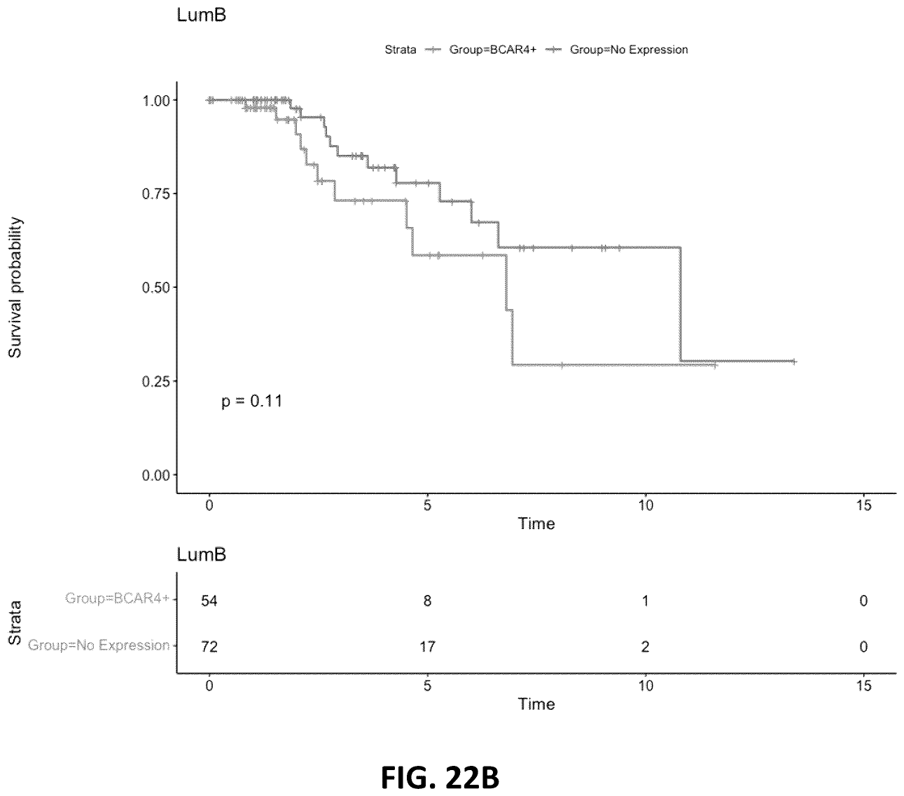

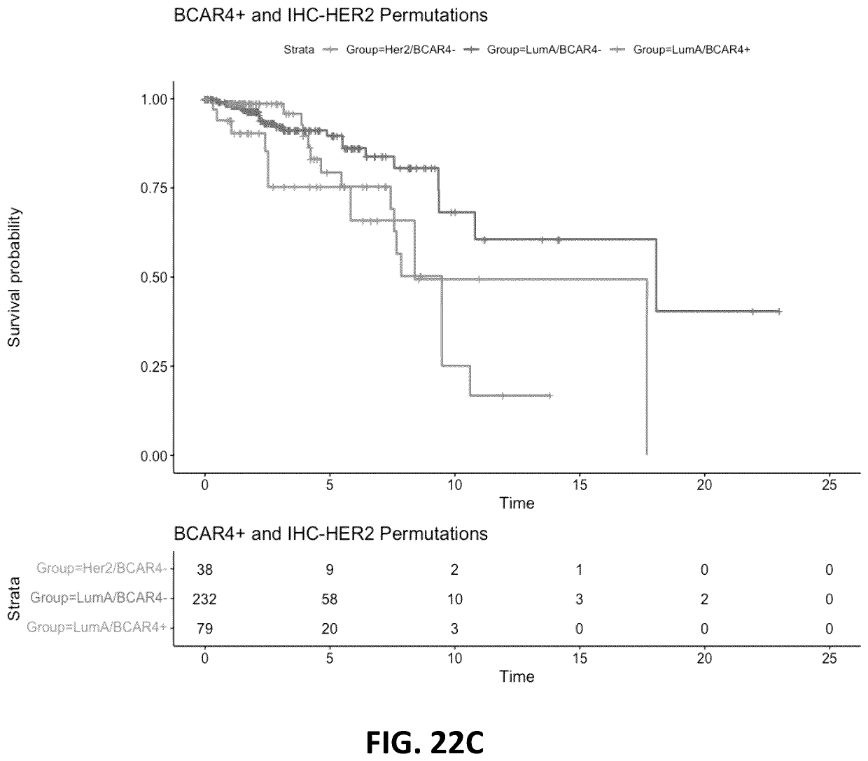

The luminal subtype is a less aggressive breast cancer disease with a distinct treatment regimen. However, when luminal-subtype patients are stratified by BCAR4 expression these BCAR4-high patients have worse overall survival of the Luminal A and Luminal B subtypes (red line; see e.g., FIG. 22A-FIG. 22C). The BCAR4-positive luminal subtype is a more aggressive disease than patients without BCAR4 in the same clinical subtype (see e.g., FIG. 22C). The BCAR4-positive luminal patients (blue line) have an overall poor survival similar to the more aggressive HER2 subtype (red line).

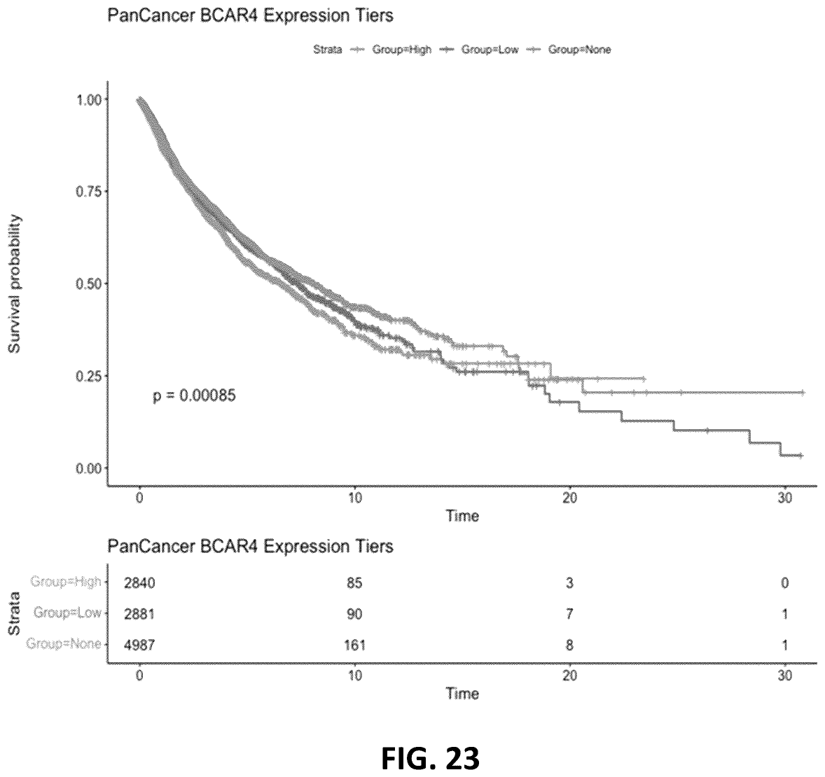

Last, expression of BCAR4 in a pan-cancer analysis across 10,708 patients indicate that high-BCAR4 expression (red line) is predictive of poor survival compared to low-BCAR4 expression or no BCAR4 expression (see e.g., FIG. 23). This patient data indicates that BCAR4 is a prognostic biomarker for breast cancer and across solid tumor types.

BCAR4 as a Predictive Biomarker

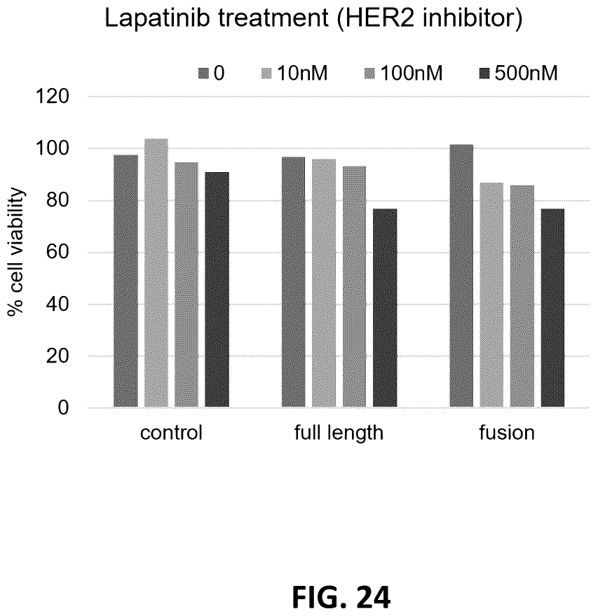

BCAR4 expression sensitizes cells to HER2-directed therapy The HER2-positive treatment landscape historically, and recently, continues to expand with new, exciting therapies improving patient outcome. There are at least 9 HER2-directed FDA-approved therapies to treat HER2-positive breast cancer; three of these therapies approved in the last two years. However, these therapies are only approved for the 15% of breast cancer patients clinically diagnosed as HER2-positive. Current clinical HER2-positive patient subtyping is based on gene amplification and protein expression through tissue staining. Without gene amplification, subtyping is scored by protein staining intensity with only the most intense (highest score) subtyped as HER2-positive. Ongoing clinical trials show patients with low HER2 expression respond well to HER2-directed therapies with a 10-month increased survival. The models described herein of low HER2 expression and overexpressed BCAR4 show sensitivity to HER2-directed treatment compared to control cells without BCAR4 expression (see e.g., FIG. 24).

The classification of breast cancers helps determine disease behavior (change of recurrence/metastasis/survival) and treatment decisions. With the advent of new therapies and ongoing clinical trials, the concept of “clinically” HER2-positive is evolving to include an additional 50% of breast cancer patients that may respond to HER2-directed therapies. The expanded definition of HER2-positive cancer also highlights the shortcomings (missed classification) of relying on the gold-standard staining for HER2 protein and the unmet clinical need for more sensitive diagnostic assays to better stratify patients for treatment. Genomic testing is a more sensitive method to determine the molecular profile of disease (across cancer types). There are at least 3 different genomic based molecular profiling assays used to clinically diagnosis and determine treatment in breast cancer patients: MammaPrint and BluePrint (Agendia) and PAM50 (Prosigna). Another company (Tempus) has a general cancer gene panel. None of these assays are monitoring BCAR4.

The landscape of HER2-positive disease is not exclusive to breast cancer but other solid tumors, which as shown herein contain documented BCAR4 gene fusion events. Ongoing clinical trials (and new FDA-approval) in gastric, colorectal, and non-small lung cancers show patients are responding well to novel HER2-directed therapies. Monitoring BCAR4 expression in these additional cancer types could stratify patients that may respond to HER2-directed therapy.

BCAR4-Positive Patients are Resistant to Hormone Therapy

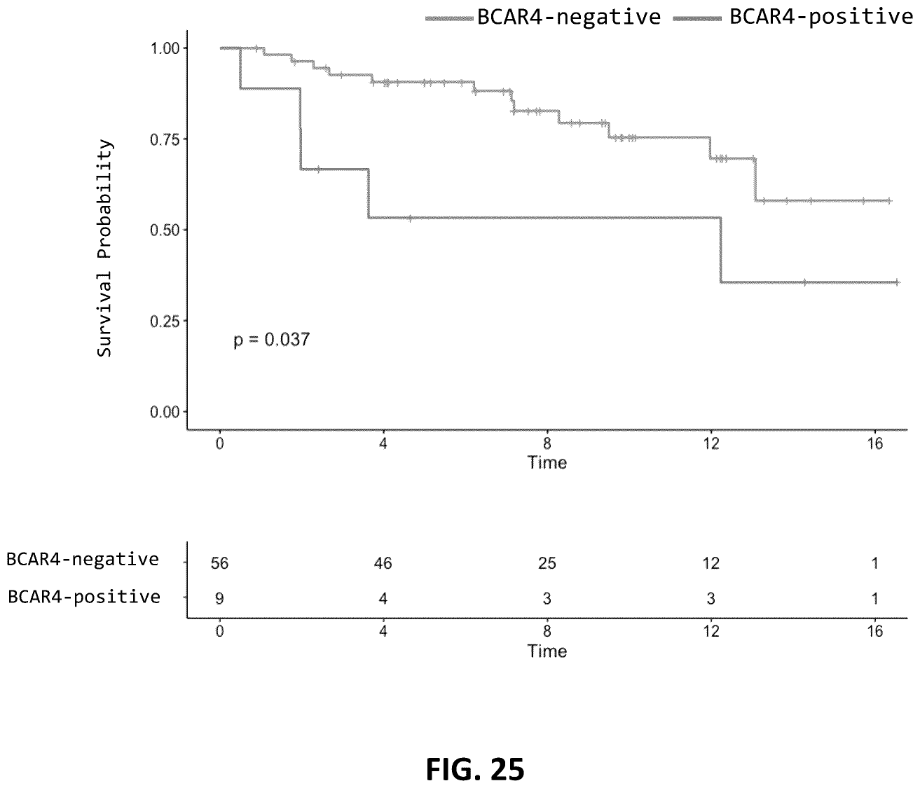

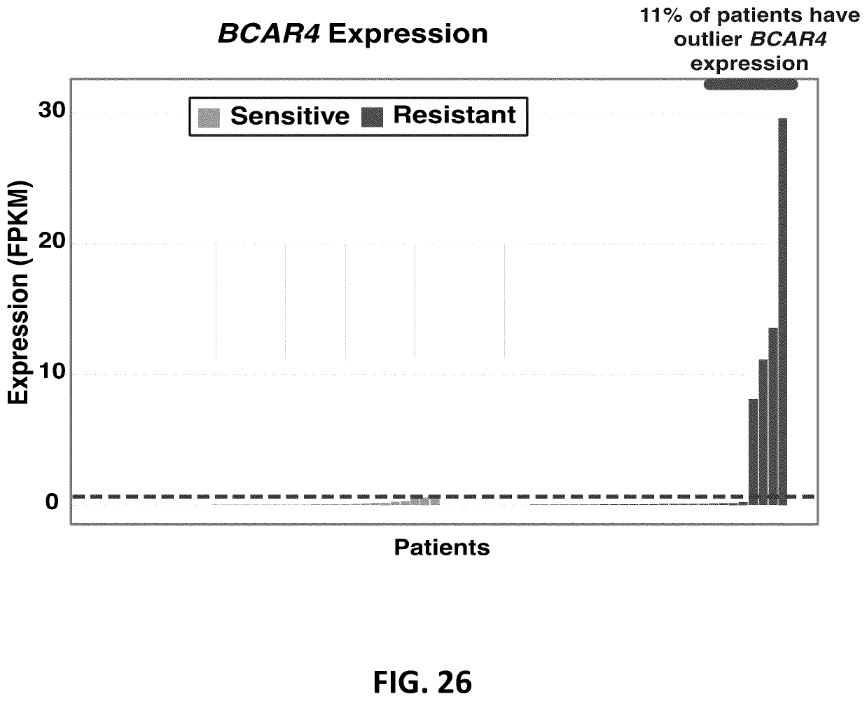

The first line treatment for Estrogen Receptor-positive (ER) breast cancer patients is hormone therapy (tamoxifen or aromatase inhibitors). Full-length BCAR4 associates with endocrine resistance in human breast cancer. In FIG. 25, the data indicate early stage BCAR4-positive (blue) patients have poor outcome when treated with tamoxifen compared to BCAR4-negative (red) patients from microarray data of early-stage breast cancer patients. Further, BCAR4 was identified as a biomarker of aromatase inhibitor (hormone therapy) resistance by performing transcriptome analysis of pretreatment tumor biopsies accrued from patients enrolled in the preoperative letrozole phase 2 study (NCT00084396) and the American College of Surgeons Oncology Group (ACOSOG) Z1031 study (NCT00265759). Recurrent BCAR4 expression (both full length and gene fusions) driving outlier expression (see e.g., FIG. 26) of BCAR4 was discovered in four patients (11%) that were resistant to hormone therapy. These data suggest identifying patients that are BCAR4-positive would indicate response to hormone therapy, which is the current standard of care for ER-positive patients.

Clinical Assay to Detect BCAR4 Expression

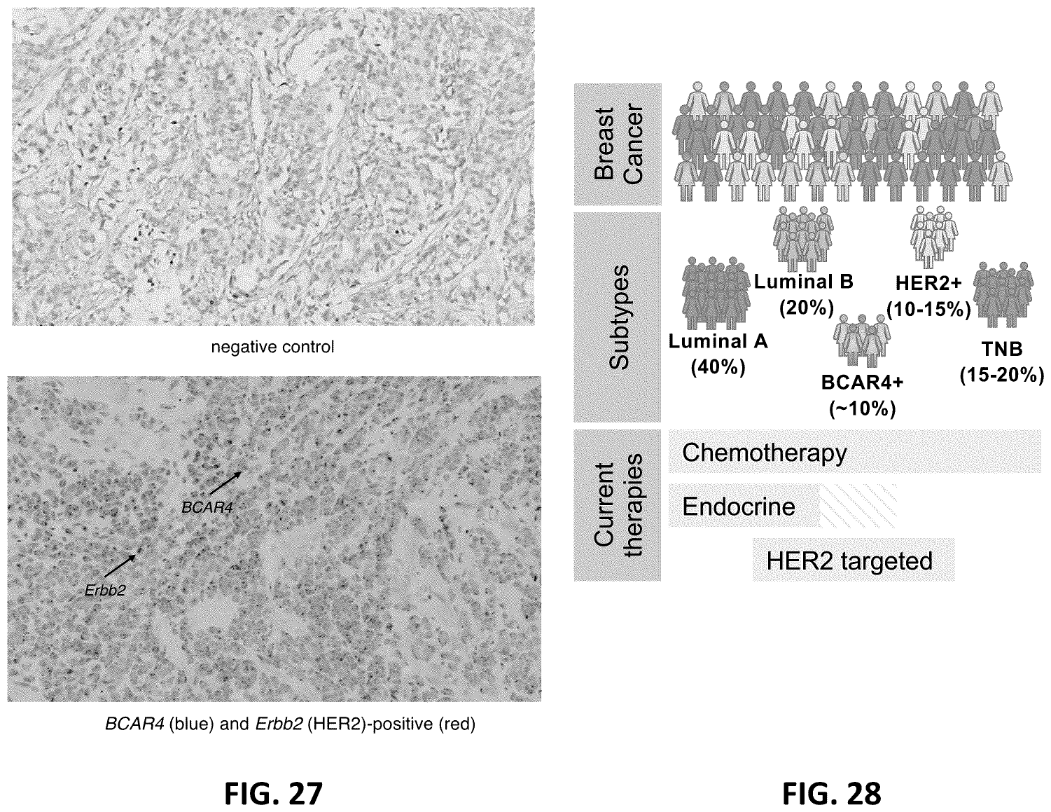

Classification of the HER2-positive subtype is undergoing a paradigm shift resulting in inclusion of more patients receiving HER2-directed therapies and the need for better diagnostic tools. HER2 protein staining is not sensitive enough to capture the HER2 low patient population. Herein is described optimization of an RNA tissue staining assay to detect BCAR4 expression (and other genes) using RNA in situ hybridization (ISH). RNA-ISH is significantly more sensitive than staining for protein. BCAR4 and Erbb2/HER2 can be detected with this technique indicating RNA-ISH as feasible to incorporate BCAR4 monitoring into clinical practice (see e.g., FIG. 27). BCAR4 expression was investigated (at the RNA level) in the 225 patient Hormonally Treated tissue microarray (TMA) curated at The St. Louis Breast Tissue Registry. Specific RNA probes are custom designed (˜20 target double-Z) for the BCAR4 probe to monitor the 4^{th} exon of the BCAR4 gene using the RNAscope 2.5 HD from ACDBio. The tissues are visualized using the ZEISS AxioImager brightfield microscope. Collectively, the data indicate that stratifying breast cancer patients by BCAR4 expression indicate a more aggressive Luminal A subgroup and worse outcome with resistant to the standard of care treatment. Further, the in vitro data indicate these patients may respond to HER2-directed therapies. This research addresses a clinical unmet need to identify BCAR4-positive patients who will respond to the growing panel of FDA-approved HER2-directed therapies and will not respond to hormone therapy (see e.g., FIG. 28).

### Example 3: Biological and Clinical Significance of Functionally Recurrent BCAR4 Gene Fusions in HER2 Non-Amplified Breast Cancer

This Example describes an experimental approach to establish BCAR4 as a predictive biomarker and develop a noninvasive assay to monitor its expression.

Introduction

Breast cancer is the most common cancer diagnosis in women worldwide. Disease heterogeneity necessitates extensive and continued molecular profiling to improve patient care. The HER2-amplified treatment landscape historically, and recently, continue to expand with new and exciting therapies improving patient outcome. However, these treatment options are currently only approved for the 15% clinically subtyped HER2-amplified (HER2+) breast cancer population. Herein is investigated a gene found to be upregulated in HER2 non-amplified patients that activates HER2/HER3 signaling. This has enormous clinical potential for identifying additional patients-beyond the HER2+ subtype-that would benefit from existing FDA-approved HER2-targeted therapies.

Clinical Relevance of Gene Fusions

Chromosomal rearrangements represent the most prevalent category of somatic aberrations in cancer genomes, juxtaposing two genes, creating gene fusions. Gene fusions are exquisitely specific diagnostic markers, prognostic indicators, and therapeutic targets. Over the last few years, Next Generation Sequencing (NGS) led to the discovery of clinically actionable gene fusions including RAF kinase fusions in melanoma, gastric, and prostate cancer, NOTCH fusions in triple negative breast cancer, ESR1 fusions in ER+ breast cancer, and ALK and ROS fusions in lung cancer. These studies exemplify the importance of identifying druggable rearrangements stratifying patients that may benefit from targeted therapies. Furthermore, while some gene fusions occur in ˜1-5% of patients per cancer type, the accumulation of these patients across multiple common solid tumors represent a significant population.

Discovery of Recurrent BCAR4 Gene Fusions Across Solid Tumors

Transcriptome sequencing data from 9,638 patients across 33 different cancer types as part of The Cancer Genome Atlas consortium was analyzed. The most prevalent gene activated was Breast Cancer Anti-estrogen Resistance 4 (BCAR4)—a known oncogene—in 11 cancer types with stomach, cervical, and breast cancers having the greatest prevalence (see e.g., FIG. 2B). BCAR4 gene fusions were prioritized because they: (1) are the most prevalent, previously uncharacterized gene fusion across solid tumors (see e.g., FIG. 2A), (2) full-length BCAR4 is known to promote aggressive disease and treatment resistance, and (3) all of the fusion events across patients express a minimum common region of BCAR4 suggesting its conserved oncogenic function(s). Two common intra-chromosomal rearrangements were observed across patients that result in the 1st untranslated exon of either ZC3H7A or LITAF juxtaposed to the 4^{th} BCAR4 exon. This results in the regulatory regions serving as a switch to activate the 4^{th} BCAR4 exon.

BCAR4 Gene Fusions Alter Cell Cycle in Benign Cells

Since full-length BCAR4 can promote tumor growth and metastasis, it was hypothesized that BCAR4 gene fusions also promote oncogenesis. Silencing gene fusions in two independent cell lines with endogenous BCAR4 gene fusion expression: SNU-308 gallbladder cancer cells (LITAF-BCAR4) and TUHR14TKB renal carcinoma cells (ZC3H7A-BCAR4) resulted in significantly fewer S-phase cells (see e.g., FIG. 4A-FIG. 4B). Next, the two most prevalent isoforms observed in patients (LITAF-BCAR4 and ZC3H7A-BCAR4; see e.g., FIG. 3 and FIG. 6) were overexpressed in two benign breast cancer cell models. Both BCAR4 gene fusions increased S-phase proliferation in HME1 and MCF10A cells relative to empty vector (see e.g., FIG. 7A-FIG. 7B, FIG. 10A-FIG. 10B, and FIG. 17A-FIG. 17B). The data demonstrate that BCAR4 gene fusions are functionally recurrent and capable of altering the cell cycle.

BCAR4 Encodes a Small Protein Important for Oncogenic Phenotypes

Conflicting studies annotate full-length BCAR4 as a long noncoding RNA (lncRNA) and a protein-coding gene. A previous study published that full-length BCAR4 functions as a lncRNA, and has multiple functional interaction sites (exon 1 and 4) to promote breast cancer metastasis. Other studies showed full-length BCAR4 encodes a protein via an open reading frame (4th exon) in oocytes and a lobular breast cancer cell. Herein was computationally predicted that the 4th BCAR4 exon contains a predicted open reading frame, which is common to both LITAF-BCAR4 and ZC3H7A-BCAR4 fusions. A mutant construct was made disrupting the ATG start site (mut LITAF-BCAR4; see e.g., FIG. 3 and FIG. 6) to determine if the protein function was necessary for oncogenesis. Benign epithelia cells expressing mut LITAF-BCAR4 had fewer S-phase cells than cells expressing wild-type fusions (see e.g., FIG. 17A-FIG. 17B). This suggests that the small protein is important for observed oncogenic phenotypes.

BCAR4 Gene Fusions in Breast Cancer

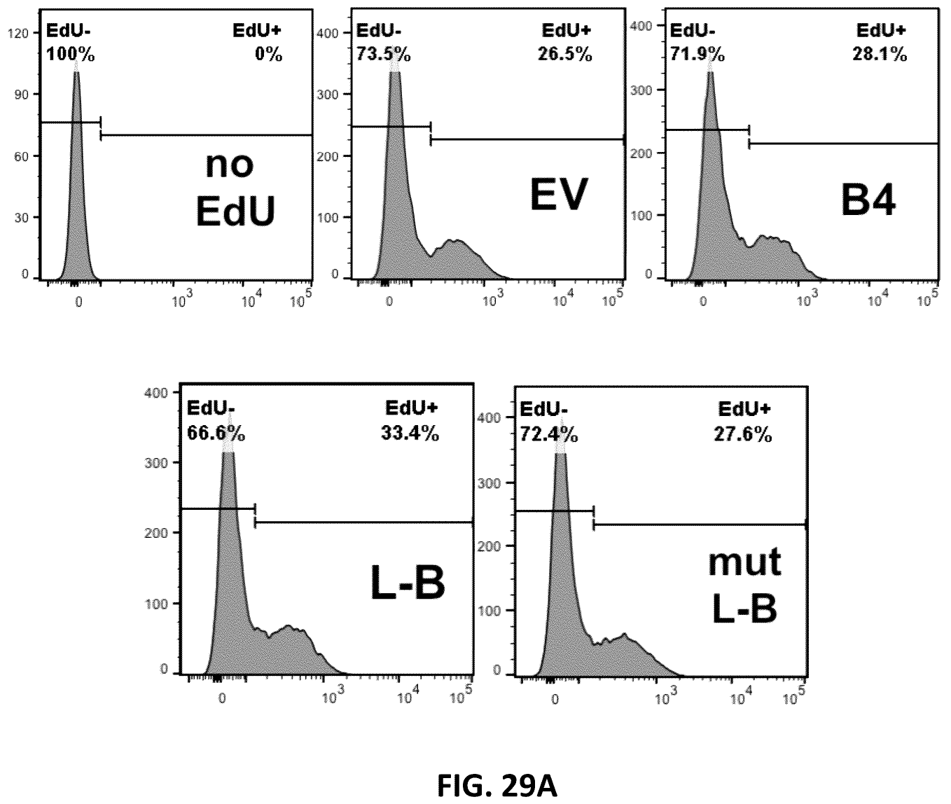

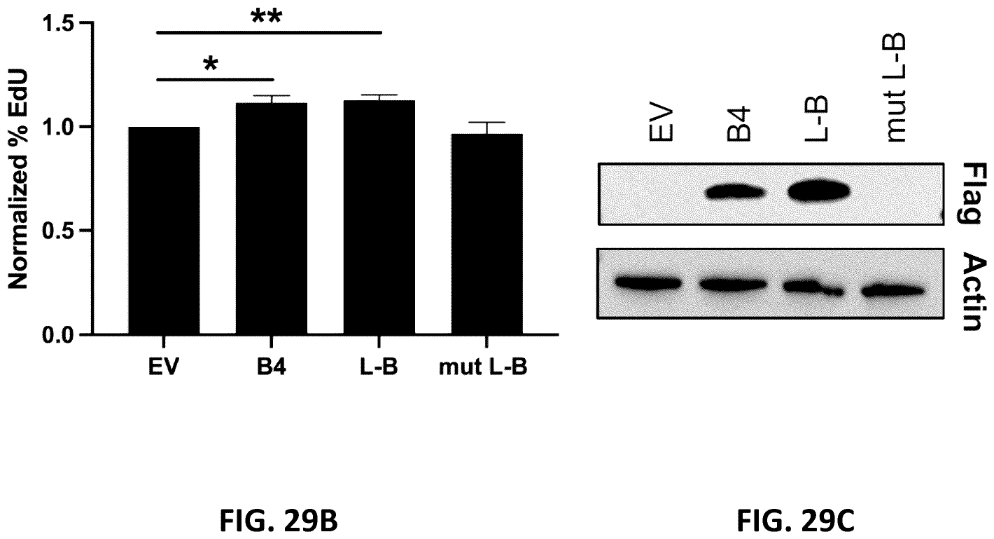

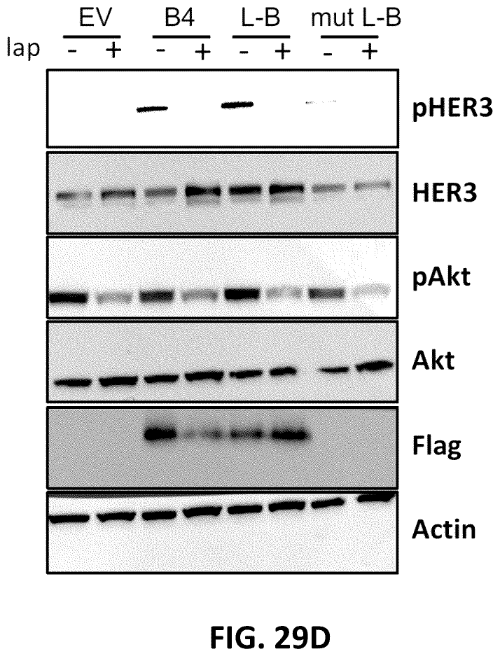

Approximately 10% of breast cancer patients in The Cancer Genome Atlas cohort (TCGA) have BCAR4 overexpression (gene fusion events and full length expression). Notably, these patients are predominately HER2-negative in the Luminal A or B subtype classification (see e.g., FIG. 20). The BCAR4 gene fusions were overexpressed in the breast cancer cell line ZR-75-1 (lacking BCAR4 and HER2 amplification) to show BCAR4-fusions cause increased proliferation, while the mutant did not (see e.g., FIG. 29A-FIG. 29C). Cells expressing full length BCAR4, or gene fusions, show phosphorylation of HER3 and downstream activation of AKT (see e.g., FIG. 29D). Further, HER2 inhibition (lapatinib) attenuated these changes. These data indicate that the gene fusions behave like full-length BCAR4. Collectively, the data demonstrate that BCAR4 gene fusions are functional, activate HER2/HER3 (in HER2 non-amplified models), sensitize cells to lapatinib, and promote aggressive disease in breast cancer.

Exploiting the Methylome to Detect BCAR4 Gene Activation

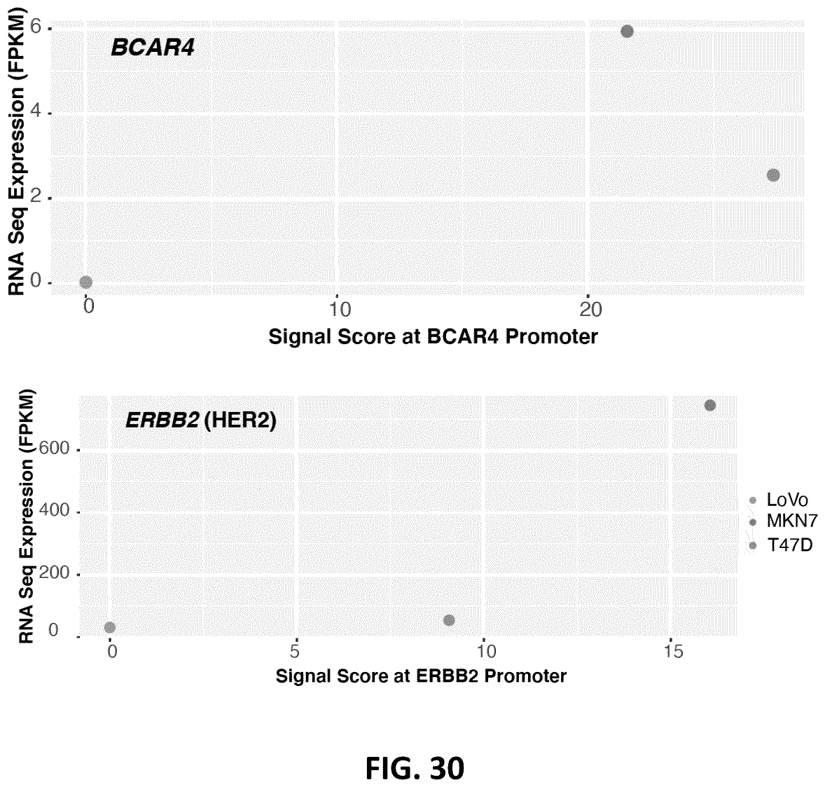

For a decade researchers have used genomic rearrangements to monitor disease in circulating cell-free DNA (cfDNA). DNA-based approaches require monitoring broad genomic regions for breakpoints and do not detect corresponding expression changes. These limitations are overcome by monitoring expressed RNA chimeras, but RNA instability make it unreliable for cell-free detection. To circumvent these limitations, an innovative technique was developed leveraging cfDNA stability and an epigenetic surrogate (5hmC) for gene expression. 5hmC DNA methylation signatures are distinct between different tissues in the body within cancer patients; this observation led to rapid development of noninvasive liquid assays to detect and monitor treatment response in cancer patients. An assay was previously developed to monitor cfDNA stratifying prostate patients with more sensitivity than current FDA-approved diagnostic tools. Herein is described the optimization of an assay to monitor gene activation (through 5hmC marks). Using cell lines with varied expression of BCAR4 and ERBB2 (HER2) a positive correlation was determined between 5hmC and gene expression (see e.g., FIG. 30). These data serve as feasibility to expand this assay into clinically relevant samples.

Specific Aims

The preliminary data are strong rationale to understand how BCAR4 gene fusions sensitize breast cancer cells to HER2-targeted therapies. The long-term goal is to establish BCAR4 as a predictive biomarker for breast cancer patient treatment and develop a noninvasive assay monitoring its expression. To accomplish this is proposed two Aims:
Aim 1. To determine how BCAR4 activates HER2/HER3 signaling and sensitizes cells to HER2-targeted treatment. The data show BCAR4 gene fusions activate HER3 through its small encoded protein. It will be determined if BCAR4 gene fusions directly or indirectly bind to HER receptor/s to cause additional oncogenic phenotypes. HER2-targeted treatment response will also be assessed in BCAR4+ patient-derived xenograft (PDX) models.
Aim 2. To optimize a noninvasive assay for identifying BCAR4-positive/HER2-negative patients that may respond to HER2-targeted therapies. The data indicate that BCAR4 gene fusions are clinically actionable. Tissue and plasma from well-annotated retrospectively banked samples will establish assay accuracy and feasibility to establish BCAR4 as a predictive biomarker to noninvasively identify patients—beyond the existing HER2-amplified population—that would benefit from HER2-targeted treatment. These Aims will generate preliminary data for a study exploring the biological and clinical significance of functionally recurrent BCAR4 gene fusions in HER2 non-amplified breast cancer. This may have enormous translational potential for noninvasive detection of BCAR4 as an innovative diagnostic biomarker to predict patient treatment response.

Aim 1. To Determine how BCAR4 Activates HER2/HER3 Signaling Sensitizing Cells to HER2-Targeted Treatment

Rationale:

The data show HER3 activation in BCAR4 gene fusion expressing cells (see e.g., FIG. 29D). Herein the mechanism will be explored by determining if this is a direct or indirect role on HER2/HER3 (preferred heterodimer) signaling and if BCAR4 gene fusions sensitize cells to lapatinib treatment (HER2 inhibition). Further, utilizing unique models it will be determined if the signaling and oncogenic phenotypes are dependent on the BCAR4 small protein.

SA1.1 to Determine Oncogenic Phenotypes of BCAR4 Gene Fusions in Breast Cancer Cell Lines.

The data show alterations in cell cycle with gene fusion overexpression in ZR-75-1 breast cancer cell line. Three (expressing FLAG) BCAR4 expression constructs have been synthesized (see e.g., FIG. 3 and FIG. 6): (1) full-length BCAR4, (2) LITAF-BCAR4 (L-B fusion), and (3) ZC3H7A-BCAR4 (Z-B fusion). Constructs will be stably over expressed by retrovirus infection in ZR-75-1 and MCF7 (lacking BCAR4 and HER2-amplification) breast cancer cells, and compared with empty vector to evaluate additional oncogenic phenotypes: migration/invasion using a Modified Boyden chamber assay; cellular viability using a MTT assay; and anchorage-independent growth using soft agar colony formation assays, as previously described.

SA1.2 to Determine if BCAR4 Gene Fusion Directly or Indirectly Regulate the HER2/HER3 Dimer.

Preliminary data show activation of HER2/HER3 and downstream signaling, a pathway extensively exploited for clinical inhibition in breast cancer. HER2/HER3 and downstream signaling will be monitored in cells overexpressing gene fusions by western blotting. BCAR4 gene fusion cellular localization will be determined (monitoring FLAG expression) using spin column separation following the Invent Technology protocol. Next, BCAR4 will be immunoprecipitated by the FLAG-tag following ThermoFisher protocols to determine if BCAR4 directly binds to HER2 or HER3.

SA1.3 to Determine if BCAR4 Gene Fusion Expression Sensitizes Cells to HER2-Targeted Therapies.

Preliminary patient data indicate BCAR4 gene fusions predominately exist in HER2-negative patient subtypes—a population not currently approved for HER2-targeted therapies. The in vitro data show activation of HER2/HER3 protein that is inhibited with lapatinib (HER2 inhibitor) treatment (see e.g., FIG. 29D). These experiments will determine if HER2 inhibition also attenuated oncogenic phenotypes. Drug titration experiments will determine BCAR4 gene fusion sensitivity to lapatinib relative to control cells. Cells will be seeded for an MTT assay as described in SA1.1 and then treated with a serial dilution (ranging from 10 nM-2.5 uM) of lapatinib to measure cell viability in response to drug. Changes in HER2/HER3 and downstream signaling will be monitored by western blot in response to lapatinib.

SA1.4: BCAR4 Gene Fusion Selectively Activate an Embedded Small Protein.

The 4th exon of BCAR4 is the common region found in all gene fusions (see e.g., FIG. 2A-FIG. 2B, FIG. 3, and FIG. 6). Unbiased mass spectrometry data supports a predicted small protein in the 4th exon of BCAR4. Protein expression is monitored in the constructs with a FLAG-tag. The data show this protein is necessary for the observed oncogenic phenotypes by using a LITAF-BCAR4 construct with a mutated start codon (see e.g., FIG. 6) with consequent loss of FLAG expression (see e.g., FIG. 29D). Leveraging this unique model, phenotype (SA1.1) and signaling changes (SA1.2) that are sensitized to lapatinib (SA1.3) can be monitored. Mutant and wild-type results will be compared to determine whether all, some, or none of the phenotypes are altered with disruption of the protein.

SA1.5 to Determine the Therapeutic Potential of BCAR4 Gene Fusions.

RNA-Seq data was used to identify BCAR4 gene fusions in two PDX models derived from ER+ breast cancer patients that lack HER2 amplification. Lapatinib treatment response will be assessed in a BCAR4+ PDX mouse model. All animal studies will be performed with Washington University's Institutional Animal Care and Use Committee approval. PDX tissue will be implanted into the flank of 12 six-week-old female NOD/SCID/yc^{-/-} (NSG) mice (Jackson Labs) to measure tumor growth. Biweekly digital caliper measurements will be used to determine the tumor size using the equation: (pi/6)(L×W2), L=length and W=width. When tumor volumes reach 1 cm, mice from each PDX line will be randomized for lapatinib or vehicle treatment. When the primary tumor becomes 2 cm or the mice become ill, they will be sacrificed and tumor will be dissected for histology, snap frozen, or cells harvested for Ki-67 staining and biomarker analysis (e.g., BCAR4, HER2).

If BCAR4 gene fusions do not bind HER receptors an unbiased approach of mass spectrometry will be performed to determine other binding partners.

The preliminary data in benign cells and breast cancer cell lines show BCAR4 gene fusions cause increased proliferation—specifically through the predicted BCAR4 protein. This Aim will determine the extent of BCAR4 driven phenotypes, whether BCAR4 interacts with the HER2/HER3 protein complex to activate downstream signaling, and cellular response to HER2-targeted therapies in BCAR4 gene fusion/HER2 non-amplified cell and PDX mouse models. Overall, this Aim will demonstrate the mechanistic and clinical significance of BCAR4 gene fusions to activate HER2/HER3 signaling and sensitize non-amplified HER2 models to HER2-targeted therapies.

Aim 2: To Optimize a Noninvasive Assay Identifying BCAR4-Positive/HER2-Negative Patients.

The HER2-positive (HER2+) breast cancer treatment landscape continues to rapidly expand with new effective therapies—but approved for 15% of the breast cancer population. Current clinical HER2+ patient subtyping is based on gene amplification and protein expression through tissue staining. Without gene amplification, subtyping is scored by protein staining intensity with only the most intense (highest score) subtyped as HER2+. It is this non-amplified, HER2 low score range—with intact signaling—that may be BCAR4-positive and may respond to HER2-targeted therapies (see e.g., FIG. 2A-FIG. 2B and FIG. 29A-FIG. 29D). Currently there is no method to identify these patients. To address this unmet clinical need, a noninvasive liquid assay could provide a more sensitive, less invasive method to detect molecular events to improve current clinical diagnosis.

SA2.1 Annotated Matched Patient Tissue and Plasma to Develop Noninvasive BCAR4 Detection.

BCAR4 5hmC DNA status—as a surrogate of gene activation—will be monitored to develop a noninvasive biomarker assay in cell-free DNA (cfDNA). Preliminary data correlate BCAR4 gene expression to 5hmC levels in cell lines (see e.g., FIG. 30). Published protocols will be used to isolate and process cfDNA (20 ng) to monitor 5hmC levels. IDT barcode indexes will be added for pooled sequencing of ˜25 million reads 75 base-pair, paired-end sequencing. Negative (BCAR4−) control samples will be included. The sequence alignment and peak detection will be performed, as done previously. The assay will be optimized with the following genomically characterized and clinically annotated cohorts:

Treatment naive tumor biopsies: Four BCAR4+ tumor biopsies and plasma will be used to optimize the assay by benchmarking 5hmC plasma levels to their matched tissue to show assay specificity.

Banked samples: Seventy-five banked late-stage breast cancer samples with matched plasma were identified based on: 1) consent, 2) ER+, and 3) HER2 non-amplification status. Now samples will be prioritized, based on HER2-targeted response to identify BCAR4+ samples (tissue) and then monitor 5hmC (plasma).

SA2.2 Monitoring BCAR4 on Prospective Patient Liquid Collection

Previous sub aims will optimize BCAR4 detection using tissue and plasma; here the clinical utility of using liquid samples to monitor BCAR4 expression with the 5hmC assay will be determined. Patient enrollment will be ongoing, but for this study 150 samples will be processed. Cf-DNA will be isolated as previously described and processed for 5hmC marks as described above. ER, HER2, and PR 5hmC levels will also be monitored to test assay performance against current clinical practices (IHC). A cohort of ˜15 BCAR4-positive samples is anticipated to prove technical feasibility of using 5hmC as a surrogate for gene activation on liquid biopsies.

Plasma samples have varying levels of cfDNA which may affect DNA yields. This may motivate optimization of the assay: collecting more blood and changing to a library prep kit using less (50 pg) of input.

Completion of this Aim will 1) optimize a noninvasive assay to monitor BCAR4 (full length and fusion) activation in patients and 2) initiate a collection protocol for breast cancer patient tissues/fluids. Data collected will establish assay sensitivity and reproducibility as proof-of-concept to innovatively monitor BCAR4 expression in patients. Future studies will use this feasibility to statistically power the prospective cohort to establish BCAR4 as a predictive biomarker to non-invasively identify patients—beyond the existing HER2 amplified population—that would benefit from HER2-targeted treatments.

Innovation

Described herein is the first study exploring the biological and clinical significance of BCAR4 gene fusions in breast cancer. Current studies accept BCAR4 functions exclusively as a noncoding RNA. Herein will be demonstrated that BCAR4 gene fusions produce a protein important for its function Xenograft models from BCAR4-gene fusion PDX breast cancer models will be used to understand if gene fusion expression sensitizes mice to HER2-targeted therapies. This is the first study to assess monitoring BCAR4 noninvasively to predict and inform on patient response and treatment options. This study has a broader impact as BCAR4 genomic alterations are indicated in 11 cancer types.

### Example 4: Methods of Measuring BCAR4 Gene Fusions for Cancer Treatment

BCAR4 expression associates with bad outcomes across cancers, breast cancer, and specifically within breast cancer subtypes. There are no systematic studies looking across solid tumors correlating BCAR4 expression and outcome. Studies have not defined a BCAR4-high subtype that could correlate with outcome.

Higher BCAR4 expression correlates with worse survival in pan-cancer analysis in TCGA cohort (see e.g., FIG. 23). High vs low is split 50-50 among samples with measurable BCAR4 expression.

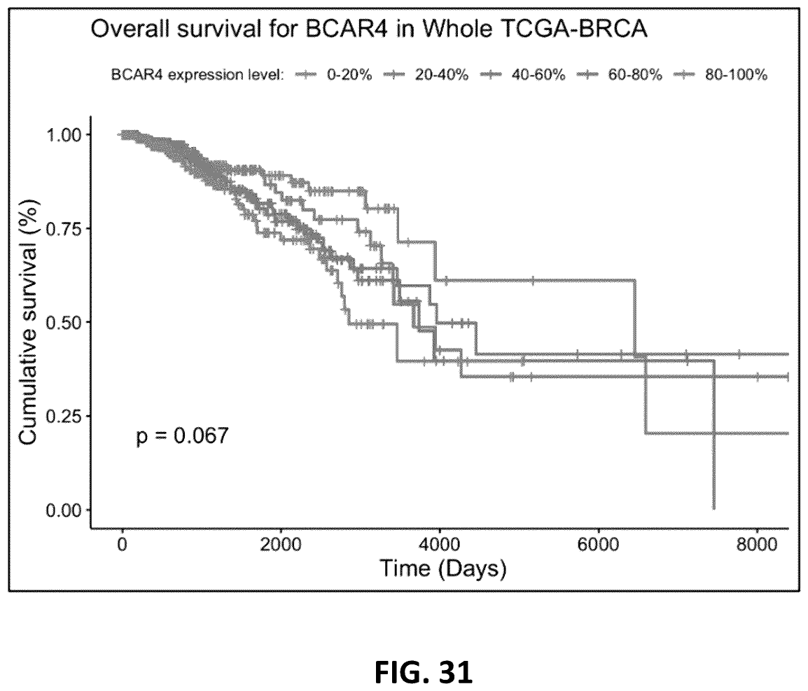

Elevated BCAR4 expression corresponds to worse overall survival in breast cancer in TCGA cohort (see e.g., FIG. 31).

PAM50 classification indicate a Luminal/BCAR4-high breast cancer population (see e.g., FIG. 20). Luminal disease is the less aggressive subtype. BCAR4 expression is mostly found in Luminal A and B patients.

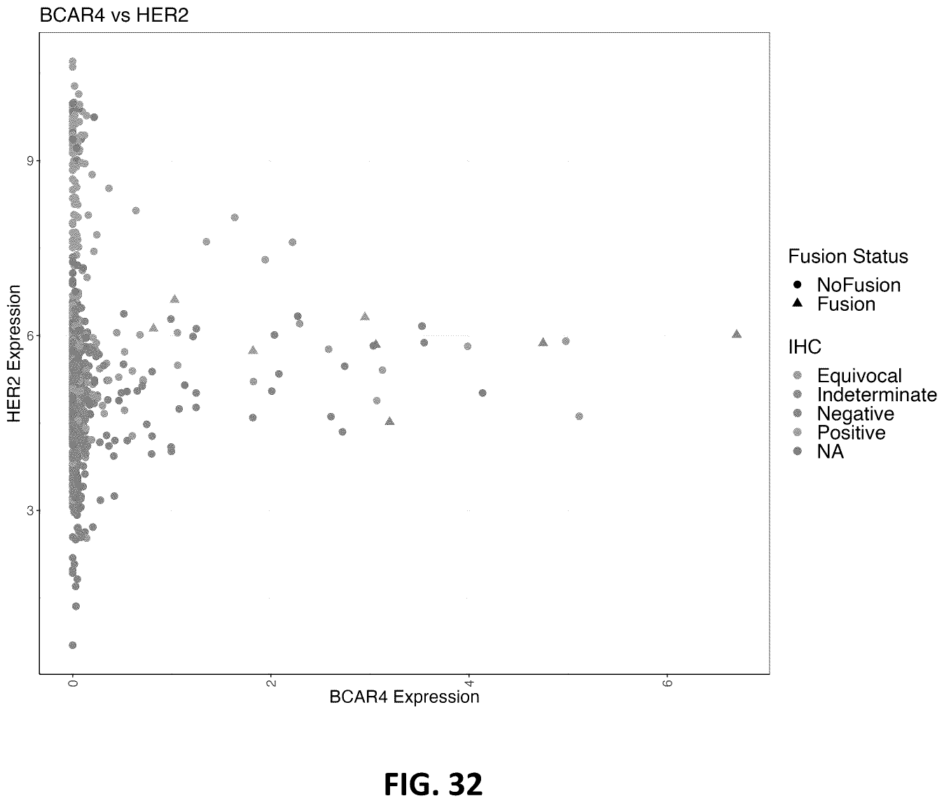

BCAR4 does not correlate with different levels of HER2 expression (see e.g., FIG. 32). The breast cancer field is very excited to understand the different efficacies of newer antibody-drug conjugates in HER2-low and HER2-negative (e.g., HER2 non-amplified luminal) patients. The data herein do not show that high BCAR4 expression correlates with immunohistochemistry (IHC) status of HER2 expression. BCAR4 full length and BCAR4 gene fusion is expressed independent of HER2 gene expression levels and IHC classification of HER2 protein levels.

BCAR4-expressing Luminal A (PAM50) patients have worse overall survival in the TCGA cohort (see e.g., FIG. 22A).

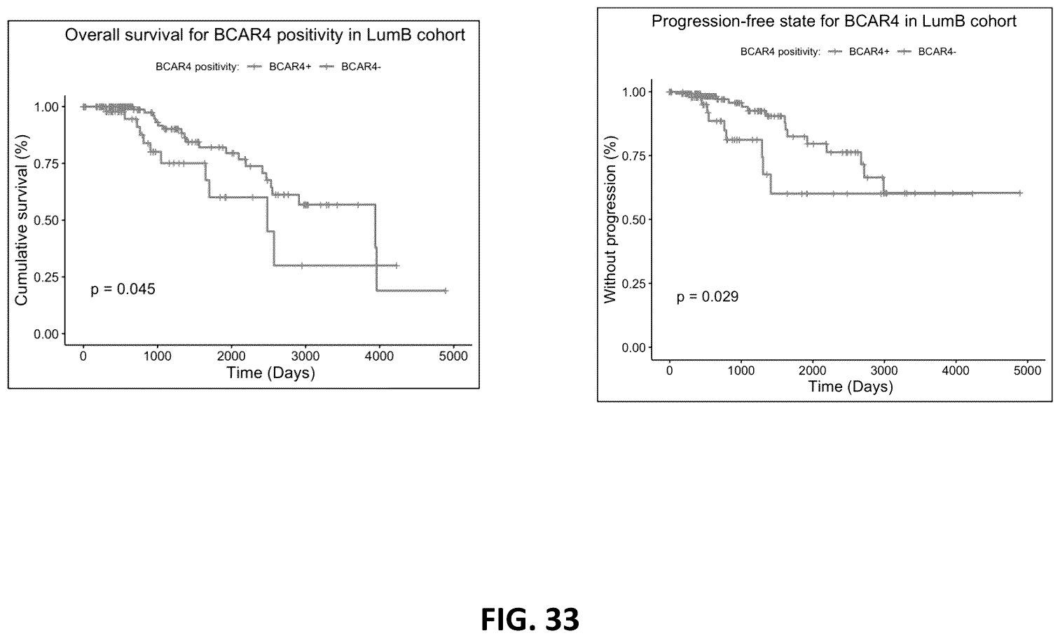

BCAR4-expressing Luminal B (PAM50) patients have worse OS and PFS in TCGA cohort (see e.g., FIG. 33).

The luminal/BCAR4-high subtype is associated with poor outcomes similar to HER2+ subtypes (see e.g., FIG. 22C). BCAR4 expression transforms nonaggressive tumors to more aggressive disease (HER2+).

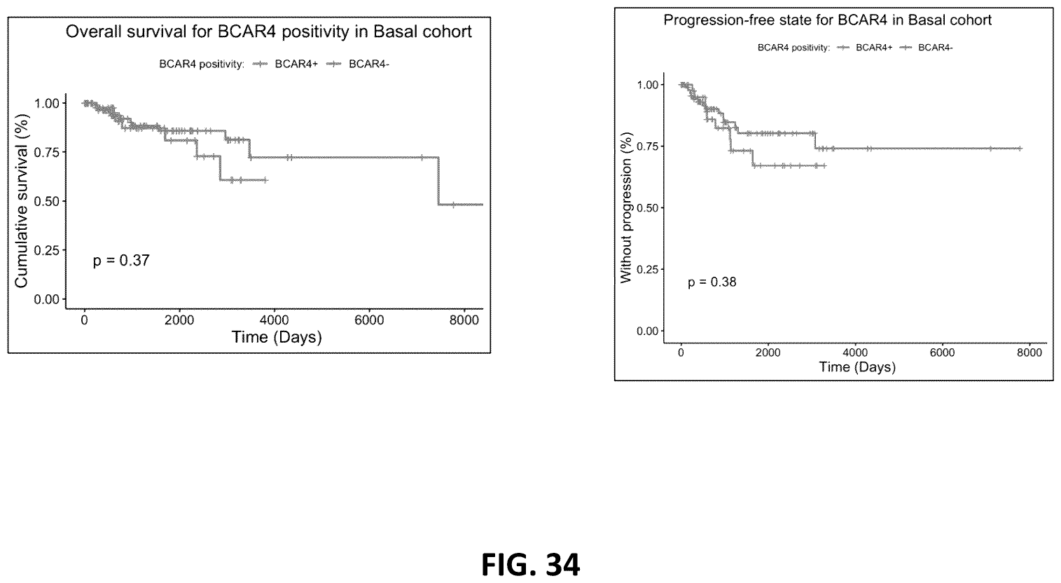

BCAR4-expressing basal (PAM50) patients trend towards worse OS and PFS in TCGA cohort (see e.g., FIG. 34).

BCAR4 gene expression contributes to hormone therapy resistance. It has been shown by qPCR that BCAR4 expression associates with tamoxifen resistance. But no previous work has shown whether BCAR4 gene fusion expression is associated with tamoxifen or newer treatments (aromatase inhibitors) treatment.

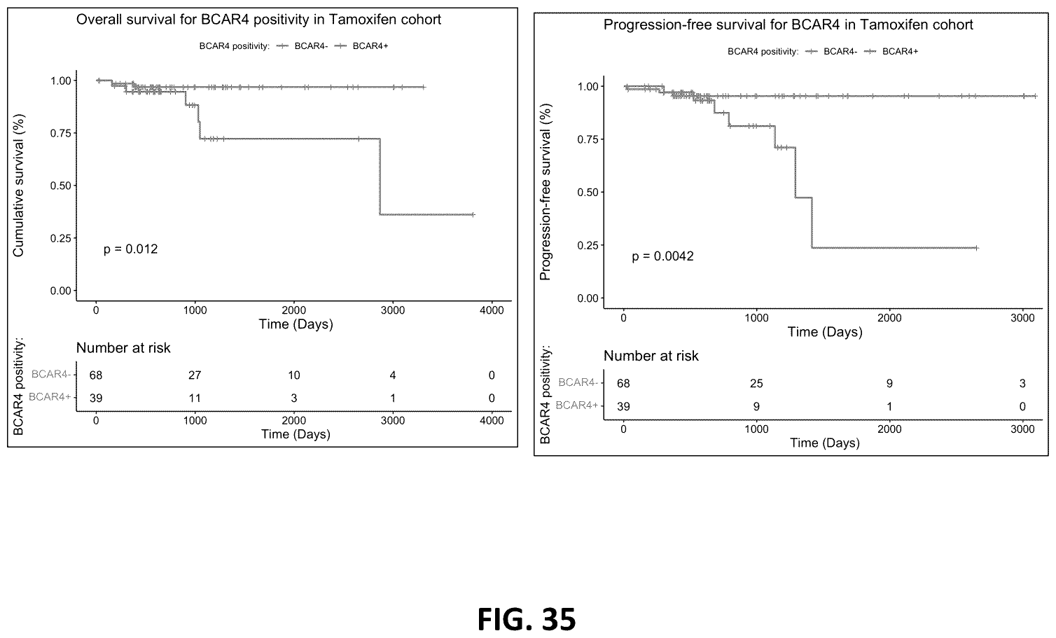

As shown herein, BCAR4-high patients that received tamoxifen only treatment had worse outcomes in TCGA cohort (n=186) (see e.g., FIG. 35). Described herein is the first study to validate the initial finding of BCAR4 expression associating with tamoxifen resistance in additional cohorts.

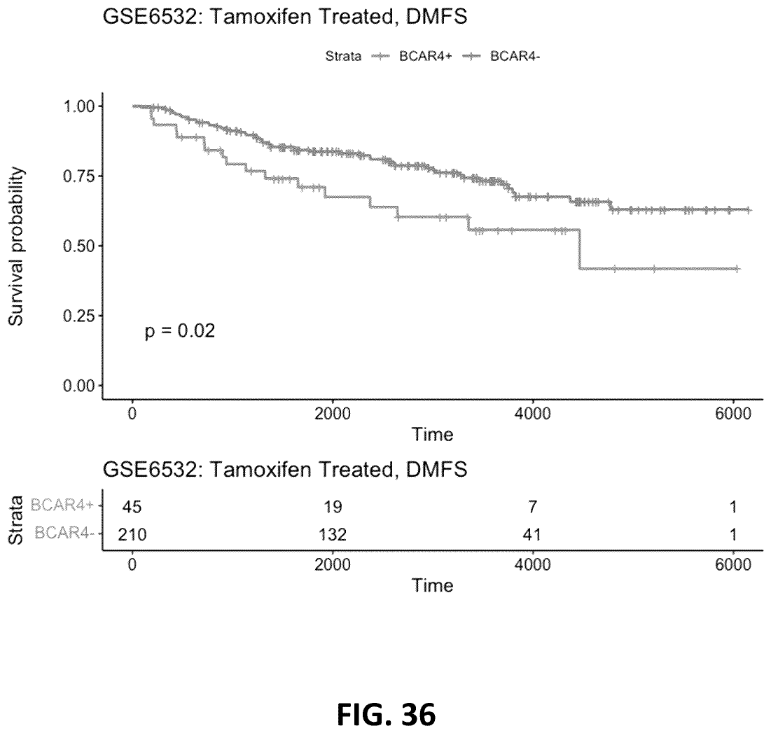

BCAR4-high patients had worse distant metastasis free survival with tamoxifen treatment in GSE6532 cohort (see e.g., FIG. 36).

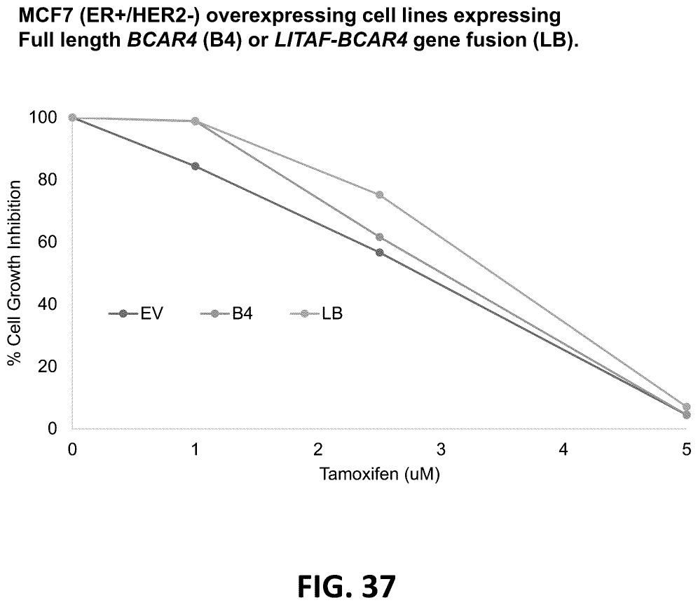

BCAR4 gene fusion-expressing cells are resistant to Tamoxifen treatment in breast cancer (see e.g., FIG. 37). It is known that full length BCAR4 is resistant to tamoxifen therapy, but no one has shown that BCAR4 gene fusions cause resistance. BCAR4-fusions express only the 4^{th} exon of BCAR4 gene. This study is the first to show that the 4^{th} exon—encoding the peptide—is sufficient to cause resistance to tamoxifen in vitro.

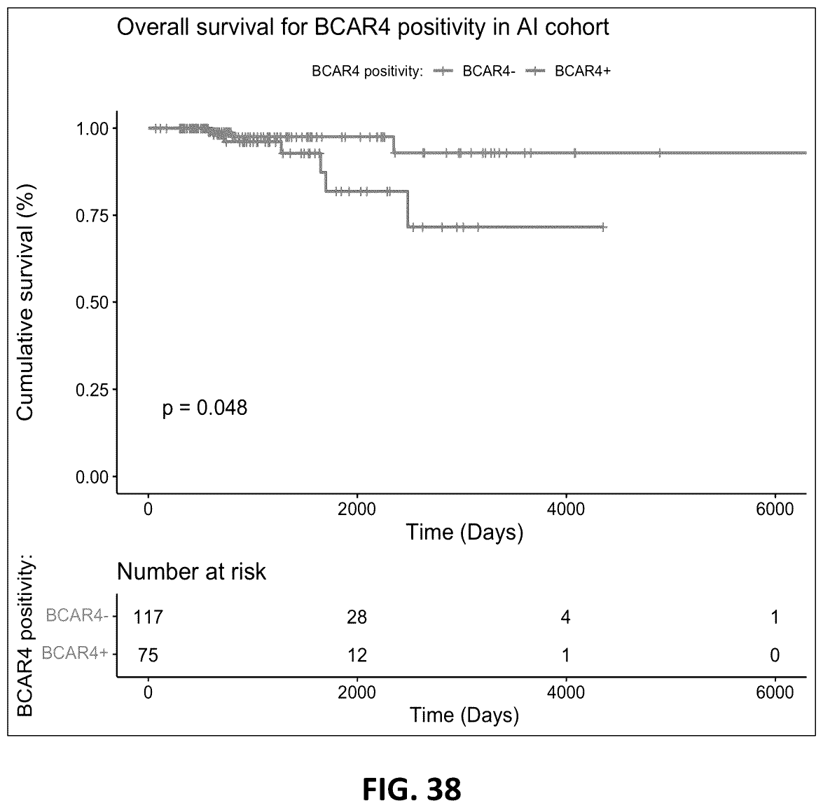

BCAR4-high patients that received Aromatase Inhibitor (AI) therapy associate with worse outcome (n=324) in TCGA cohort (see e.g., FIG. 38). Described herein is the first study to show that high BCAR4 expression associates with AI resistance. AI treatment is a newer therapy to block hormone production.

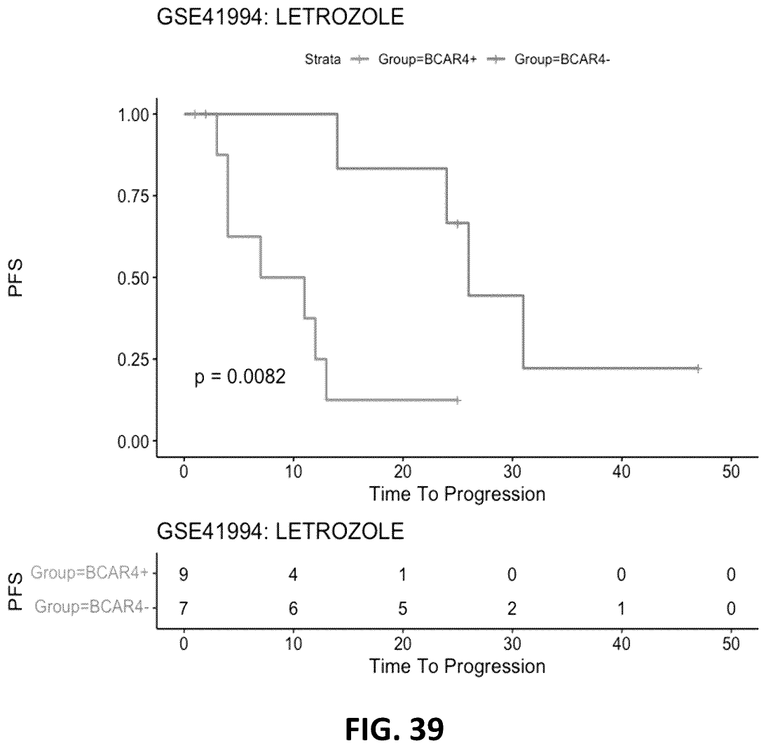

Patients expressing BCAR4 are resistant to aromatase inhibitor (AI) therapy (see e.g., FIG. 39). Described herein is the first study to show that expression of BCAR4 gene fusion or full length BCAR4 gene cause resistance to aromatase inhibitor (AI) therapy. AI treatment is the next generation of hormone therapy for ER+ breast cancer patients.

BCAR4 gene fusion expression was identified as a biomarker for aromatase inhibitor (hormone therapy) resistance by performing transcriptome analysis of pretreatment tumor biopsies accrued from patients enrolled in the preoperative letrozole phase 2 study (NCT00084396) and the American College of Surgeons Oncology Group (ACOSOG) Z1031 study (NCT00265759) (see e.g., FIG. 26). Recurrent BCAR4 gene fusions driving outlier expression of BCAR4 were discovered in four patients (11%) that were resistant to hormone therapy.

Available public data (GSE41994) showed that the top 25% of patients expressing BCAR4 have worse Progression Free Survival (PFS) on letrozole (AI treatment) (see e.g., FIG. 39).

BCAR4 protein is localized to the plasma membrane and directly interacts with HER2 receptor to activate it. It has been shown that BCAR4 associates with HER2 activation by western blot. However, described herein is the first study to show where the protein localizes in the cell and that it directly interacts with HER2 at the plasma membrane.

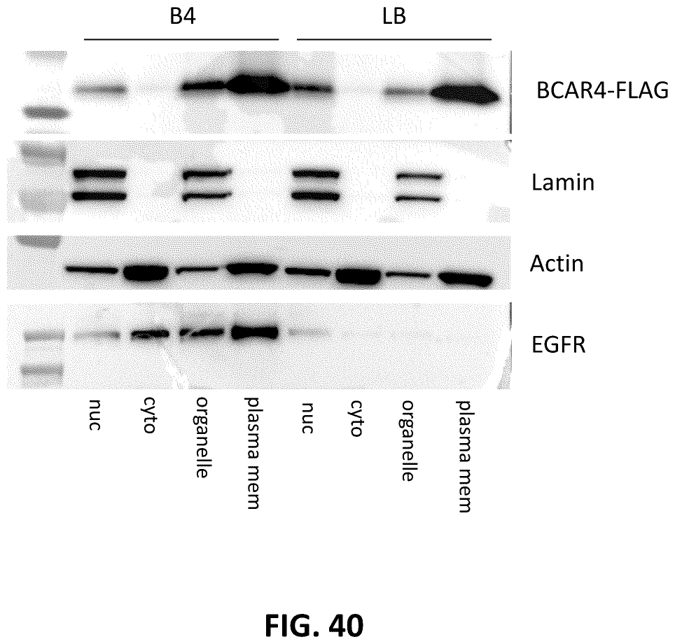

BCAR4 protein localizes to the plasma membrane of breast cancer cells (see e.g., FIG. 40). Cellular isolation shows different cell compartments. Described herein is the first study to show the localization of full length BCAR4 and BCAR4-gene fusions in the plasma membrane.

Protein-protein interactions may be studied using proximity labeling technic (TurboID).

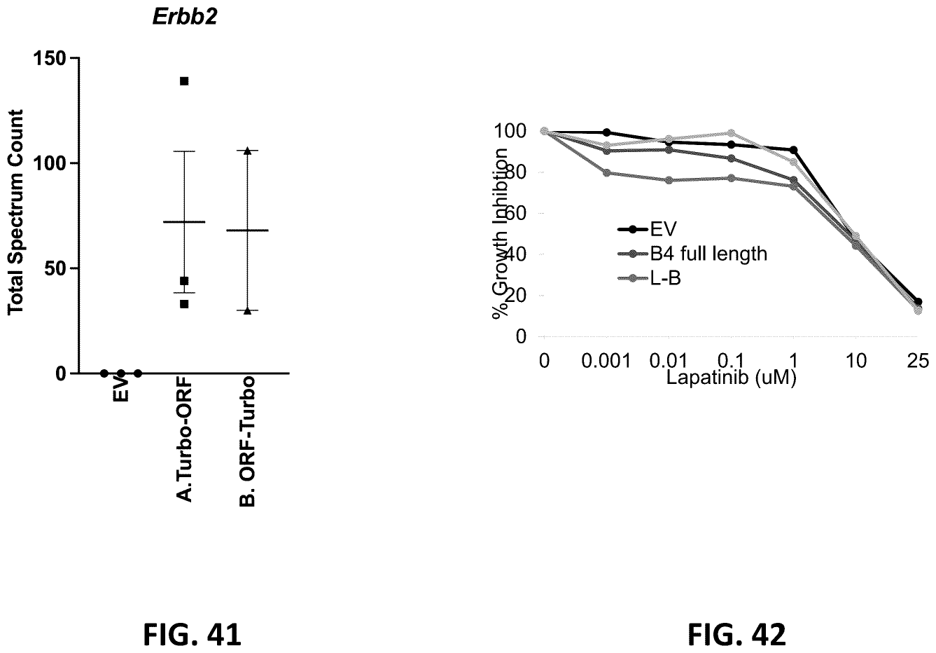

As shown herein, BCAR4 directly interacts with HER2 (Erbb2) (see e.g., FIG. 41). Described herein is the first study to show the direct interaction of BCAR4 protein with the HER2 receptor with proximity labeling and unbiased mass spectrometry analysis.

BCAR4 protein causes cells to respond to HER2-targeted therapies. It has been shown that full length BCAR4-expressing cells respond to lapatinib treatment. However, shown herein is that BCAR4 gene fusion-expressing cells also respond to lapatinib. Also shown herein is that this response is dependent on the encoded peptide with the 4^{th} exon of full length BCAR4. Last, this study is the first to show that BCAR4 gene fusion-expressing cells respond to another form of HER2 inhibition—Herceptin (trastuzumab).

Cells respond to lapatinib (HER2 inhibitor) in the presence of BCAR4 protein (see e.g., FIG. 29D and FIG. 42). Active HER3 (pHER3) was observed in cells expressing BCAR4 full-length or gene fusions (see e.g., FIG. 29D). In breast cancer HER3 preferentially binds to HER2 to form a heterodimer. Indeed, BCAR4-expressing cells have decreased growth when treated with lapatinib (see e.g., FIG. 42). Lapatinib-treated cells lose HER3 activation (see e.g., FIG. 29D). Collectively, these data support functional BCAR4 gene fusions that activate HER2/3 (in HER2-low models), respond to lapatinib, and promote aggressive disease in breast cancer. Using the unique BCAR4 protein mutant, this study is the first to demonstrate that it is the protein function that is necessary to elicit a response to lapatinib treatment.

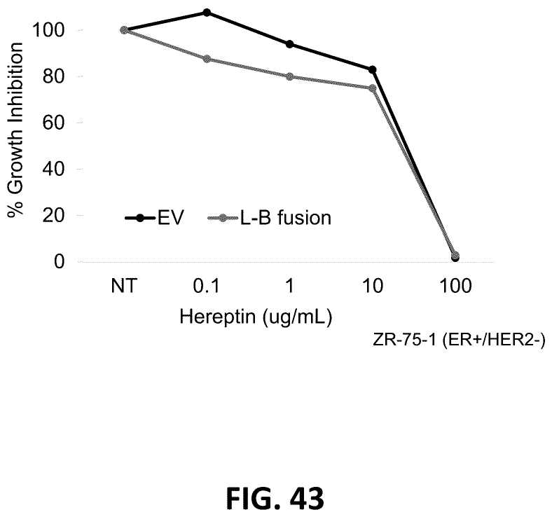

BCAR4-expressing cells respond to Herceptin (trastuzumab) (see e.g., FIG. 43). Herceptin, a monoclonal antibody treatment that revolutionized HER2-positive patient treatment, was found to be ineffective in HER2-low patients (PMID 31821109). Interestingly, the preliminary data herein show BCAR4 gene fusion (L-B fusion) expression increased cell response to Herceptin in cell growth (MTT) assays.

What is claimed is:

1. A method of treating a subject having a HER2 non-amplified (HER2-negative) cancer or low HER2 expressing (HER2-low) cancer, the method comprising:
providing a biological sample from the subject;
detecting expression of a BCAR4 gene fusion in the biological sample; and
administering:
a HER2-targeted cancer treatment to the subject if expression of the BCAR4 gene fusion is detected, or
a cancer treatment that is not HER2-targeted to the subject if expression of the BCAR4 gene fusion is not detected.

2. The method of claim 1, wherein the BCAR4 gene fusion comprises a nucleotide sequence derived from LITAF, ZC3H7A, or a variant thereof.

3. The method of claim 1, wherein the BCAR4 gene fusion comprises exon 4 of the BCAR4 gene or a variant thereof.

4. The method of claim 1, wherein the BCAR4 gene fusion comprises a nucleotide sequence encoding a peptide comprising SEQ ID NO: 1, SEQ ID NO: 2, or a variant thereof.

5. The method of claim 1, wherein the subject has a HER2-negative or HER2-low cancer selected from the group consisting of stomach cancer, cervical cancer, breast cancer, esophageal cancer, ovarian cancer, skin cancer, bladder cancer, lung cancer, uterine cancer, colon cancer, or prostate cancer.

6. The method of claim 5, wherein the subject has HER2-negative or HER2-low breast cancer.

7. The method of claim 6, wherein the subject has a luminal A or luminal B breast cancer.

8. The method of claim 1, wherein the HER2-targeted cancer treatment comprises lapatinib or trastuzumab.

9. A method for predicting response to a HER2-targeted cancer treatment in a subject having a HER2 non-amplified (HER2-negative) cancer or low HER2 expressing (HER2-low) cancer, the method comprising:
providing a biological sample from the subject;
detecting expression of a BCAR4 gene fusion in the biological sample; and
determining:
the subject is predicted to be responsive to a HER2-targeted cancer treatment if expression of the BCAR4 gene fusion is detected, or
the subject is predicted to be unresponsive to a HER2-targeted cancer treatment if expression of the BCAR4 gene fusion is not detected.

10. The method of claim 9, wherein the BCAR4 gene fusion comprises a nucleotide sequence derived from LITAF, ZC3H7A, or a variant thereof.

11. The method of claim 9, wherein the BCAR4 gene fusion comprises exon 4 of the BCAR4 gene or a variant thereof.

12. The method of claim 9, wherein the BCAR4 gene fusion comprises a nucleotide sequence encoding a peptide comprising SEQ ID NO: 1, SEQ ID NO: 2, or a variant thereof.

13. The method of claim 9, wherein the subject has a HER2-negative or HER2-low cancer selected from the group consisting of stomach cancer, cervical cancer, breast cancer, esophageal cancer, ovarian cancer, skin cancer, bladder cancer, lung cancer, uterine cancer, colon cancer, or prostate cancer.

14. The method of claim 13, wherein the subject has HER2-negative or HER2-low breast cancer.

15. The method of claim 9, wherein the HER2-targeted cancer treatment comprises lapatinib or trastuzumab.

16. The method of claim 9, wherein if expression of the BCAR4 gene fusion is detected, the subject is further predicted to be resistant to a hormone therapy.

17. A method of detecting BCAR4 or BCAR4 gene fusion activation in a subject having cancer, the method comprising:
providing a biological sample from a subject;
isolating cell-free DNA (cfDNA) from the biological sample; and
measuring a 5-Hydroxymethylcytosine (5hmC) signal for BCAR4 in the cfDNA using sequencing, wherein the 5hmC signal positively correlates with BCAR4 or BCAR4 gene fusion expression.

18. The method of claim 17, wherein the 5hmC signal is measured at the BCAR4 promoter.

19. The method of claim 17, wherein the biological sample comprises plasma.

20. The method of claim 17, wherein the subject has a HER2 non-amplified (HER2-negative) cancer or low HER2 expressing (HER2-low) cancer.


SEQUENCE LISTING (21 sequences)

SEQ ID NO: 1 - protein, 15 aa, Homo sapiens
MYQPIQTYPWMNLSR

SEQ ID NO: 2 - protein, 22 aa, Homo sapiens
KSGSLQGTTEPSMTHSIIASTS

SEQ ID NO: 3 - protein, 121 aa, Homo sapiens
MYQPIQTYPWMNLSRRREFRCLSCSECLLVTCLGLSTVILGLIVVLQDPSDSVVFSTGLTMIAIGAFFVVLTGVTALCTVTVDENLQKTTRLRLGVIRKSGSLQGTTEPSMTHSIIASTSL

SEQ ID NO: 4 - RNA, 19 nt, synthetic construct
  source 1..19: mol_type = other RNA
gactaggagtgatacgaaa

SEQ ID NO: 5 - RNA, 19 nt, synthetic construct
  source 1..19: mol_type = other RNA
tttcgtatcactcctagtc

SEQ ID NO: 6 - RNA, 19 nt, synthetic construct
  source 1..19: mol_type = other RNA
cgctgtagttgtacattga

SEQ ID NO: 7 - RNA, 19 nt, synthetic construct
  source 1..19: mol_type = other RNA
tcaatgtacaactacagcc

SEQ ID NO: 8 - DNA, 20 bp, synthetic construct
  source 1..20: mol_type = other DNA
aggcattgacaacagggttc

SEQ ID NO: 9 - DNA, 20 bp, synthetic construct
  source 1..20: mol_type = other DNA
gttgcacatcagcagcactt

SEQ ID NO: 10 - DNA, 18 bp, synthetic construct
  source 1..18: mol_type = other DNA
cgagaggccagctcagac

SEQ ID NO: 11 - DNA, 20 bp, synthetic construct
  source 1..20: mol_type = other DNA
tcagagcaagacaagcatcg

SEQ ID NO: 12 - DNA, 19 bp, synthetic construct
  source 1..19: mol_type = other DNA
gctaaccgagggagagctg

SEQ ID NO: 13 - DNA, 20 bp, synthetic construct
  source 1..20: mol_type = other DNA
ggaactcccgtcttctggat

SEQ ID NO: 14 - DNA, 24 bp, synthetic construct
  source 1..24: mol_type = other DNA
gggagataccatgatcacgaaggt

SEQ ID NO: 15 - DNA, 26 bp, synthetic construct
  source 1..26: mol_type = other DNA
ccacaaattatgcagtcgagtttccc

SEQ ID NO: 16 - DNA, 20 bp, synthetic construct
  source 1..20: mol_type = other DNA
gacggaggttgagatgaagc

SEQ ID NO: 17 - DNA, 20 bp, synthetic construct
  source 1..20: mol_type = other DNA
attcggggctctgtagtcct

SEQ ID NO: 18 - DNA, 20 bp, synthetic construct
  source 1..20: mol_type = other DNA
ctcgacaccagggcgttatg

SEQ ID NO: 19 - DNA, 21 bp, synthetic construct
  source 1..21: mol_type = other DNA
ccactccatgctcgataggat

SEQ ID NO: 20 - protein, 16 aa, Homo sapiens
MYQPIQTYPWMNLSRR

SEQ ID NO: 21 - protein, 22 aa, Homo sapiens
SGSLQGTTEPSMTHSIIASTSL